SEQUENCE LISTING (16 sequences)

SEQ ID NO: 1 - protein, 698 aa, Homo sapiens
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 2 - protein, 733 aa, Homo sapiens
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 3 - protein, 698 aa, Homo sapiens; identical to SEQ ID NO: 1

SEQ ID NO: 4 - protein, 660 aa, Homo sapiens
GGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 5 - protein, 732 aa, Artificial Sequence
  source: /note=“Description of Artificial Sequence Synthetic polypeptide”
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 6 - protein, 698 aa, Artificial Sequence; identical to SEQ ID NO: 1
  source: /note=“Description of Artificial Sequence Synthetic polypeptide”

SEQ ID NO: 7 - protein, 660 aa, Artificial Sequence
  source: /note=“Description of Artificial Sequence Synthetic polypeptide”
MGGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGFFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 8 - protein, 147 aa, Artificial Sequence
  source: /note=“Description of Artificial Sequence Synthetic polypeptide”
SAMGGVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 9 - protein, 200 aa, Artificial Sequence
  source: /note=“Description of Artificial Sequence Synthetic polypeptide”
SAMGALVGLGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 10 - protein, 60 aa, Artificial Sequence
  source: /note=“Description of Artificial Sequence Synthetic polypeptide”
GIPPAAAAKAAKYGAAGLGGVLGGAGQFPLGGVAARPGFGLSPIFPGGACLGKACGRKRK

SEQ ID NO: 11 - protein, 47 aa, Artificial Sequence
  source: /note=“Description of Artificial Sequence Synthetic polypeptide”
GAAGLGGVLGGAGQFPLGGVAARPGFGLSPIFPGGACLGKACGRKRK

SEQ ID NO: 12 - protein, 34 aa, Artificial Sequence
  source: /note=“Description of Artificial Sequence Synthetic polypeptide”
GADEGVRRSLSPELREGDPSSSQHLPSTPSSPRV

SEQ ID NO: 13 - protein, 34 aa, Artificial Sequence
  source: /note=“Description of Artificial Sequence Synthetic polypeptide”
GADEGVRRSLSPELREGDPSSSQHLPSTPSSPRF

SEQ ID NO: 14 - protein, 216 aa, Artificial Sequence
  source: /note=“Description of Artificial Sequence Synthetic polypeptide”
AAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 15 - protein, 183 aa, Artificial Sequence
  source: /note=“Description of Artificial Sequence Synthetic polypeptide”
AAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 16 - protein, 5 aa, Unknown
  source: /note=“Description of Unknown Tropoelastin motif”
GRKRK

(19) United States
(12) Patent Application Publication
(10) Pub. No.: US 2019/0275087 A1
(43) Pub. Date: Sep. 12, 2019

(54) EXPANSION AND DIFFERENTIATION OF STEM CELLS

(71) Applicant: Allergan Pharmaceuticals International Limited, Dublin (IE)

(72) Inventors: Giselle C. YEO, Sydney (AU); Anthony S. WEISS, Sydney (AU)

(21) Appl. No.: 16/289,579

(22) Filed: Feb. 28, 2019

(30) Foreign Application Priority Data
Mar. 1, 2018 (AU) 2018900663

(51) Int. Cl. A61K 35/32; C12N 5/077; C12N 5/0775; C12N 5/00

(52) U.S. Cl. CPC A61K 35/32; C12N 5/0654; C12N 5/0663; C12N 2501/105; C12N 5/0655; C12N 2533/80; C12N 2501/115; C12N 5/0037

(57) ABSTRACT

The disclosure relates to the expansion and differentiation of mesenchymal stem cells and bone marrow cells, including retention of stem cell plasticity during expansion and differentiation of stem cells to produce osteocytes, chondrocytes and other cells of the mesodermal lineage.

### CROSS REFERENCE

This application claims the benefit of Australian Provisional Patent Application Number 2018900663, filed on Mar. 1, 2018, the entire disclosure of which is incorporated herein by this specific reference.

### FIELD

The disclosure relates to the expansion and differentiation of mesenchymal stem cells and bone marrow cells, including retention of stem cell plasticity during expansion and differentiation of stem cells to produce osteocytes, chondrocytes and other cells of the mesodermal lineage.

### BACKGROUND

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Mesenchymal stem cells (MSCs) and differentiated cells derived from same such as osteocytes, adipocytes and chondrocytes are increasingly being used in therapeutic interventions for skeletal tissue injuries, myocardial infarctions, degenerative diseases, and organ failure due to their inherent differentiation and regenerative potential, immunomodulatory properties, and migratory capacity towards sites of injury and disease. However, a significant hurdle hindering widespread translation into clinical practice is the limited natural availability of these cells. For instance, human bone marrow derived MSCs comprise only 0.001-0.01% of the bone marrow mononuclear cell population. In contrast, a therapeutic dose for a single patient typically requires at least one to two million cells per kilogram of body weight, due in part to the inefficient homing of administered MSCs. Ex vivo culture for the purpose of producing osteocytes, adipocytes or chondrocytes may require isolation of approximately 1×10⁵ to 10⁶ MSCs, depending on the nature of the indication to be treated. Evidently, there is strong demand for the ability to expand MSCs cost-effectively, while maintaining stem cell properties closely linked with therapeutic efficacy. There is also a demand for the ability to differentiate MSCs ex vivo whether subject to prior ex vivo expansion or not.

The expansion and differentiation of MSCs, and other adherent therapeutic stem cells in general, relies on interactions with soluble components in the culture medium, the surrounding cells, and the underlying substrate. These factors are acknowledged to function synergistically but not redundantly, such that an underlying substrate protein would not be expected to replace a soluble component. Accordingly, MSC expansion ex vivo has been enhanced by fortifying culture media with exogenous soluble factors, and/or by coating culture surfaces with serum. For example, MSCs propagation can be amplified by supplementing basal media with additional serum proteins, hormones, or growth factors. Amongst these growth factors are transforming growth factor beta (TGF-β), epidermal growth factor (EGF), platelet-derived growth factor (PDGF), insulin-like growth factor-1 (IGF-1) and most commonly, basic fibroblast growth factor (bFGF). In particular, bFGF has a potent mitogenic effect towards MSCs, and is frequently used to supplement stem cell culture media with full or minimal serum content.

Extra-cellular matrix components in the form of fibronectin, collagen IV, vitronectin, and laminin have been predominantly used as culture substrates for the purpose of retaining cells on substrate surfaces, and are commonly used in concert with serum- or growth factor-supplemented media, the latter to promote MSC adhesion, spreading and expansion.

Holst et al. (Nat Biotech 28, 1123-1128 (2010)) cultured mouse bone marrow cells and human hemopoietic stem cells with tropoelastin and described a proliferative effect requiring monomeric tropoelastin. Substrate elasticity and tensegrity were described as important for observance of the proliferative effect. Addition of expansion cytokines with tropoelastin produced an additive effect.

Hu et al. (Biomaterials 31 8121-8131) described a structural protein blend system based on silkworm silk fibroin and recombinant human tropoelastin that forms an insoluble film. The system promotes human mesenchymal stem cell attachment and proliferation. Pure silk or pure tropoelastin cultures produced fewer cell numbers than the system.

Hu (Biomaterials 32, 8979-8989 (2011)) described the capacity of the same system to promote attachment, proliferation and myogenic or osteogenic differentiation of MSCs. Proliferation and osteogenic differentiation of MSCs required high surface roughness with micro/nano-scale surface patterns. Tropoelastin concentration did not affect the amount of hMSC proliferation.

Hu (Adv. Funct. Mater. 23, 3875-3884 (2013)) further described the same system as a ‘protein alloy’, whereby it is the alloy itself and the insoluble beta-sheet crystal network which provides for the conformation and stability required for the effects on cell morphology and growth.

Calabrese (J. Tissue Eng Regen Med 11: 2549-2564 (2017)) further studied the protein alloy in the form of a hydrogel whereby the alloy was used to encapsulate MSCs. In contrast to the earlier work by Hu, high content of tropoelastin in the alloy was found to inhibit the differentiation potential of MSCs, even in the presence of differentiation media. Calabrese supra described that tropoelastin downregulates hMSC differentiation.

### SUMMARY

In a first aspect, a method for forming cells of mesodermal lineage from mesenchymal stem cells (MSC) is provided. The method comprises the steps: contacting MSCs with (i) at least one differentiation factor for inducing formation of cells of mesodermal lineage from MSC; and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises: (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the tropoelastin is cross-linked to the hyaluronic acid. In some embodiments, the cell of mesodermal lineage is an osteocyte, chondrocyte or adipocyte. In some embodiments, the MSCs are human MSCs.

In a second aspect, a composition of cells is provided, wherein the cells are formed by a method according to any one of the embodiments herein. The cells of the composition are formed by a method for forming cells of mesodermal lineage from mesenchymal stem cells (MSC). The method comprises the steps: contacting MSCs with (i) at least one differentiation factor for inducing formation of cells of mesodermal lineage from MSC; and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises: (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the tropoelastin is cross-linked to the hyaluronic acid. In some embodiments, the cell of mesodermal lineage is an osteocyte, chondrocyte or adipocyte. In some embodiments, the MSCs are human MSCs. In some embodiments of the composition, the composition is a substantially pure form of osteocytes. In some embodiments of the composition, the composition includes tropoelastin and/or hyaluronic acid. In some embodiments, the tropoelastin is cross-linked to the hyaluronic acid.

In a third aspect, a method for treating an individual having a bone disorder or fracture, is provided. The method comprises the steps of providing a composition according to any one of the embodiments provided herein to the individual, thereby treating the individual for a bone disorder or fracture. The cells of the composition are formed by a method for forming cells of mesodermal lineage from mesenchymal stem cells (MSC). The method comprises the steps: contacting MSCs with (i) at least one differentiation factor for inducing formation of cells of mesodermal lineage from MSC; and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises: (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the tropoelastin is cross-linked to the hyaluronic acid. In some embodiments, the cell of mesodermal lineage is an osteocyte, chondrocyte or adipocyte. In some embodiments, the MSCs are human MSCs. In some embodiments of the composition, the composition is a substantially pure form of osteocytes. In some embodiments of the composition, the composition includes tropoelastin and/or hyaluronic acid. In some embodiments, the tropoelastin is cross-linked to the hyaluronic acid. In some embodiments, the individual is provided the composition, wherein the amount of total MSC provided to the individual in the composition is at least one to two million cells per kilogram of body weight of the individual. In some embodiments, the individual is provided the composition, wherein at least one to two million cells are administered to a local site.

In a fourth aspect, a cell culture medium comprising tropoelastin is provided, wherein the cell culture medium does not contain insulin-like growth factor-1 (IGF-1) and/or basic fibroblast growth factor growth factor (bFGF). In some embodiments, the cell culture medium comprises about 2.5 μg/mL to about 20 μg/mL tropoelastin. In some embodiments, the cell culture medium comprises about 2% to about 10% serum. In some embodiments, the cell culture medium comprises about 2% to about 6% serum. In some embodiments, the serum is fetal bovine serum (FBS). In some embodiments, the cell culture medium is serum-free. In some embodiments, the cell culture medium comprises minimal essential medium (MEM). In some embodiments, the cell culture medium comprises L-glutamine. In some embodiments, the cell culture medium comprises about 2.5 μg/mL to about 20 μg/mL tropoelastin, about 2% to about 10% FBS, minimal essential medium (MEM), and L-glutamine. In some embodiments, the tropoelastin is provided in the form of a complex with hyaluronic acid. In some embodiments, the tropoelastin is cross-linked to the hyaluronic acid.

In a fifth aspect, a cell culture medium comprising tropoelastin, wherein the medium does not contain a factor for inducing expansion or proliferation of MSCs is provided. In some embodiments, the cell culture medium is absent of TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the cell culture medium is absent of insulin-like growth factor-1 (IGF-1) and/or basic fibroblast growth factor growth factor (bFGF). In some embodiments, the cell culture medium comprises about 2.5 μg/mL to about 20 μg/mL tropoelastin. In some embodiments, the cell culture medium comprises about 2% to about 10% serum. In some embodiments, the cell culture medium comprises about 2% to about 6% serum. In some embodiments, the serum is fetal bovine serum (FBS). In some embodiments, the cell culture medium is serum-free. In some embodiments, the cell culture medium comprises minimal essential medium (MEM). In some embodiments, the cell culture medium comprises L-glutamine. In some embodiments, the cell culture medium comprises about 2.5 μg/mL to about 20 μg/mL tropoelastin, about 2% to about 10% FBS, minimal essential medium (MEM), and L-glutamine. In some embodiments, the tropoelastin is provided in the form of a complex with hyaluronic acid. In some embodiments, the tropoelastin is cross-linked to the hyaluronic acid.

In a sixth aspect, a cell culture is provided, wherein the cell culture comprises mesenchymal stem cells; and a medium comprising tropoelastin, wherein the medium does not contain insulin-like growth factor-1 (IGF-1) and/or basic fibroblast growth factor growth factor (bFGF). In some embodiments, the mesenchymal stem cells are human mesenchymal stem cells. In some embodiments, the medium comprises about 2.5 μg/mL to about 20 μg/mL tropoelastin. In some embodiments, the tropoelastin is provided in the form of a complex with hyaluronic acid. In some embodiments, the tropoelastin is provided in the form of a complex with hyaluronic acid. In some embodiments, the medium comprises about 2% to about 10% serum or about 2% to about 6% serum. In some embodiments, the medium is serum-free. In some embodiments, the medium comprises about 2.5 μg/mL to about 20 μg/mL tropoelastin, about 2% to about 10% FBS, minimal essential medium (MEM), and L-glutamine.

In a seventh aspect, a cell culture medium is provided, wherein the cell culture medium comprises at least one differentiation factor; and tropoelastin. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline. the tropoelastin is provided in the form of a complex with hyaluronic acid. In some embodiments, the tropoelastin is cross-linked to the hyaluronic acid.

In an eighth aspect, a cell culture comprising: mesenchymal stem cells; and a medium comprising tropoelastin, wherein the medium does not contain a factor for inducing expansion or proliferation of MSCs, is provided. In some embodiments, the factor for inducing expansion or proliferation of MSCs comprises TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the mesenchymal stem cells are human mesenchymal stem cells. In some embodiments, the medium comprises about 2.5 μg/mL to about 20 μg/mL tropoelastin. In some embodiments, the tropoelastin is provided in the form of a complex with hyaluronic acid. In some embodiments, the tropoelastin is cross-linked to the hyaluronic acid. In some embodiments, the medium comprises about 2% to about 10% serum or about 2% to about 6% serum. In some embodiments, the medium is serum-free. In some embodiments, the medium comprises about 2.5 μg/mL to about 20 μg/mL tropoelastin, about 2% to about 10% FBS, minimal essential medium (MEM), and L-glutamine.

In a ninth aspect, a cell culture is provided, wherein the cell culture comprises mesenchymal stem cells; and a medium comprising tropoelastin and at least one differentiation factor. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline. In some embodiments, the tropoelastin is provided in the form of a complex with hyaluronic acid. In some embodiments, the tropoelastin is cross-linked to the hyaluronic acid.

In a tenth aspect, a method for culturing a mesenchymal stem cell is provided, the method comprising: a) culturing a mesenchymal stem cell in a cell culture medium, wherein the medium does not contain a factor for inducing expansion or proliferation of MSCs; and b) expanding the mesenchymal stem cell in the presence of tropoelastin. In some embodiments, the mesenchymal stem cell is exposed to tropoelastin from days 1-7, days 2-5, or days 4-7 of a seven-day expansion period. In some embodiments, the factor for inducing expansion or proliferation of MSCs comprises TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the mesenchymal stem cells are human mesenchymal stem cells. In some embodiments, the medium comprises about 2.5 μg/mL to about 20 μg/mL tropoelastin. In some embodiments, the tropoelastin is provided in the form of a complex with hyaluronic acid. In some embodiments, the tropoelastin is cross-linked to the hyaluronic acid. In some embodiments, the medium comprises about 2% to about 10% serum. In some embodiments, the medium is serum-free. In some embodiments, the method further comprises differentiating the mesenchymal stem cells in a medium comprising at least one differentiation factor.

In some embodiments, a method for forming cells of mesodermal lineage from mesenchymal stem cells (MSC) is provided, the method comprises contacting MSCs with: (i) at least one differentiation factor for inducing formation of cells of mesodermal lineage from MSC; and (ii) tropoelastin; wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline. In some embodiments, the culture may typically include at least one differentiation factor for inducing formation of cells of mesodermal lineage from MSCs.

In some embodiments, a method for forming cells of mesodermal lineage from MSCs is provided, the method comprises (i) contacting MSCs with tropoelastin to induce proliferation of MSCs, thereby forming a population of MSCs; and (ii) contacting the population of MSCs with at least one differentiation factor for inducing formation of cells of mesodermal lineage from MSC and tropoelastin, thereby forming cells of mesodermal lineage from MSCs. In some embodiments, contacting MSCs with tropoelastin is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, contacting MSCs with tropoelastin is performed in the absence of IGF-1 or bFGF. In some embodiments, the culture may typically include at least one differentiation factor for inducing formation of cells of mesodermal lineage from MSCs.

In some embodiments, a method for forming cells of mesodermal lineage from MSCs is provided, the method comprising (i) providing a cell culture vessel having a cell culture surface, the cell culture surface having tropoelastin arranged thereon, said arrangement enabling a cell to contact tropoelastin arranged on the cell culture surface during cell culture when the cell is cultured on the cell culture surface; and (ii) culturing MSCs in the culture vessel in conditions enabling culture of the cell on the cell culture surface, thereby forming cells of mesodermal lineage from MSCs. In some embodiments, the method further comprises providing at least one differentiation factor for inducing formation of cells of mesodermal lineage from MSC. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline. In some embodiments, the culture may typically include at least one differentiation factor for inducing formation of cells of mesodermal lineage from MSCs.

In some embodiments, a method for forming cells of mesodermal lineage from MSCs is provided, wherein the method comprises (i) providing a cell culture vessel having a cell culture surface, the cell culture surface having tropoelastin arranged thereon, said arrangement enabling tropoelastin to at least partially dissolve in a cell culture medium for culture of an MSC; and (ii) culturing MSCs in the culture vessel, thereby forming cells of mesodermal lineage from MSCs. In some embodiments, the method further comprises providing at least one differentiation factor for inducing formation of cells of mesodermal lineage from MSC. In some embodiments, the at least one differentiation factor comprises dexamethasone, ascorbate and/or beta glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline. In some embodiments, the culture may typically include at least one differentiation factor for inducing formation of cells of mesodermal lineage from MSCs.

In some embodiments, a method for forming cells of mesodermal lineage from MSCs is provided, wherein the method comprises culturing MSCs in a cell culture medium containing solubilized tropoelastin, thereby forming cells of mesodermal lineage from MScs. In some embodiments, the method further comprises adding at least one differentiation factor for inducing formation of cells of mesodermal lineage from MSC into the cell culture medium. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline. In some embodiments, the culture may typically include at least one differentiation factor for inducing formation of cells of mesodermal lineage from MSCs.

In some embodiments, the at least one differentiation factor comprises dexamethasone, ascorbate and/or beta-glycerophosphate.

In some embodiments, the at least one differentiation factor comprises h-insulin, dexamethasone, indomethacin and/or 3-isobutyl-1-methyl-xanthine.

In some embodiments, the at least one differentiation factor comprises dexamethasone, ascorbate, insulin-transferrin-selenium, sodium pyruvate and/or proline.

In another embodiment, a method for forming cells of mesodermal lineage from MSCs is provided, wherein the method comprises culturing MSCs in a cell culture medium containing solubilized tropoelastin and at least one differentiation factor for inducing differentiation of an MSC, thereby forming cells of mesodermal lineage from MSCs. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

In another embodiment, a method for forming cells of mesodermal lineage from MSCs is provided, wherein the method comprises (i) culturing MSCs in a first medium containing tropoelastin to form a tropoelastin-cultured population; and thereafter, (ii) culturing said tropoelastin-cultured population in a second medium including at least one differentiation factor for inducing formation of cells of mesodermal lineage from an MSC, thereby forming cells of mesodermal lineage from MSCs. In some embodiments, culturing MSCs in the first medium is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, culturing MSCs in the first medium is performed in the absence of IGF-1 and bFGF. In some embodiments, tropoelastin improves MSC propagation and may be used to replace IGF-1 and/or bFGF and may be used to maintain an amplified level of cell expansion. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

In another embodiment, a method for forming cells of mesodermal lineage from MSCs is provided, wherein the method comprises culturing MSCs in a cell culture medium containing a complex of hyaluronic acid and tropoelastin, thereby forming cells of mesodermal lineage from MSCs. The culture may include at least one differentiation factor for inducing formation of cells of mesodermal lineage from MSCs. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

In one embodiment, a method for inducing proliferation of MSCs is provided, wherein the method comprises contacting MSCs with tropoelastin to induce proliferation of MSCs, wherein the number of MSCs formed in the presence of tropoelastin is greater than the number of MSCs formed in the absence of tropoelastin, thereby inducing proliferation of MSCs. In some embodiments, the method is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the method is performed in the absence of in the absence of IGF-1 and bFGF.

In another embodiment, a method for inducing proliferation of MSCs is provided, wherein the method comprises (i) providing a cell culture vessel having a cell culture surface, the cell culture surface having tropoelastin arranged thereon, said arrangement enabling a cell to contact tropoelastin arranged on the cell culture surface during cell culture when the cell is cultured on the cell culture surface, and (ii) culturing MSCs in the culture vessel in conditions enabling culture of the cell on the cell culture surface, thereby inducing proliferation of MSCs. In some embodiments, the method further comprises providing at least one proliferation factor. In some embodiments, the at least one proliferation factor is tropoelastin and/or fetal bovine serum.

In another embodiment, a method for inducing proliferation of MSCs is provided, wherein the method comprises providing a cell culture vessel having a cell culture surface, the cell culture surface having tropoelastin arranged thereon, said arrangement enabling tropoelastin to at least partially dissolve in a cell culture medium for culture of an MSC and culturing MSCs in the culture vessel, thereby inducing proliferation of MSCs. In some embodiments, the method further comprises providing at least one proliferation factor. In some embodiments, the at least one proliferation factor is tropoelastin and/or fetal bovine serum.

In another embodiment, a method for inducing proliferation of MSCs is provided, wherein the method comprises culturing MSCs in a cell culture medium containing solubilized tropoelastin thereby inducing proliferation of MSCs. In some embodiments, the method further comprises providing at least one proliferation factor. In some embodiments, the at least one proliferation factor comprises tropoelastin and/or fetal bovine serum.

In another embodiment, a method for inducing proliferation of MSCs is provided, wherein the method comprises culturing MSCs in a cell culture medium containing solubilized tropoelastin and at least one factor for inducing proliferation of an MSC thereby inducing proliferation of MSCs. In some embodiments, the at least one proliferation factor comprises tropoelastin and/or fetal bovine serum.

In another embodiment, a method for inducing proliferation of MSCs is provided, wherein the method comprises (i) culturing MSCs in a first medium containing tropoelastin to form a tropoelastin-cultured MSC population; and thereafter, (ii) culturing said tropoelastin-cultured MSC population in a second medium wherein the second medium comprises at least one proliferation factor for inducing proliferation of an MSC, thereby inducing proliferation of MSCs. In some embodiments, the at least one proliferation factor comprises tropoelastin and/or fetal bovine serum. In some embodiments, the method is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the method is performed in the absence of in the absence of IGF-1 and bFGF.

In another embodiment, a method for inducing proliferation of MSCs is provided, wherein the method comprises culturing MSCs in a cell culture medium containing a complex, wherein the complex comprises hyaluronic acid and tropoelastin, thereby inducing proliferation of MSCs.

In the above described embodiments relevant to the inducing proliferation of MSCs, the cell culture medium typically may not include a factor for inducing formation of cells of mesodermal lineage from MSCs.

In the above described embodiments, it will be understood that tropoelastin is not provided for culture in the form of an insoluble complex, or in the form of an insoluble complex or composition with another molecule, silk protein being one example.

In the above described embodiments, it will be understood that tropoelastin is generally provided in a form enabling the tropoelastin to at least partially or completely solubilize in the solvent forming the cell medium. In some embodiments, the tropoelastin is provided in a concentration wherein the tropoelastin is partially soluble in the solvent.

In the above described embodiments, it will be understood that tropoelastin may be provided in the form of a complex with hyaluronic acid that is partially or completely soluble, wherein the tropoelastin monomers are linked together by hyaluronic acid. In some embodiments, the tropoelastin is cross-linked to the hyaluronic acid.

In another embodiment, a cell culture including cells of mesodermal lineage formed by execution of any one of the above described methods for forming cells of mesodermal lineage from MSCs is provided.

In another embodiment, a method of treating an individual for a condition that requires MSCs or cells of mesodermal lineage for treatment is provided, the method comprises executing of any one of the above described methods to form a composition of MSCs or a composition of cells of mesodermal lineage and providing said composition to an individual to enable treatment of the condition, thereby treating the individual for a condition that requires MSCs or cells of mesodermal lineage for treatment.

In another embodiment, a method for forming a cell of mesodermal lineage from an MSC is provided, wherein the method comprises (i) contacting MSCs with tropoelastin to form a composition of MSCs and tropoelastin; and (ii) administering the composition to an individual requiring formation of cells of mesodermal lineage from an MSC, thereby forming a cell of mesodermal lineage from an MSC.

In another embodiment, a method for forming a cell of mesodermal lineage from an MSC is provided, wherein the method comprises (i) contacting MSCs with tropoelastin to induce proliferation of MSCs, thereby forming a composition of MSCs and tropoelastin; and thereafter, (ii) administering the composition to an individual requiring formation of cells of mesodermal lineage from an MSC, thereby forming a cell of mesodermal lineage from an MSC. In some embodiments, the contacting is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the contacting is performed in the absence of in the absence of IGF-1 and bFGF.

In another embodiment, a method for forming a cell of mesodermal lineage from an MSC is provided, wherein the method comprises (i) administering tropoelastin to an individual requiring formation of cells of mesodermal lineage from an MSC thereby forming a depot of tropoelastin in the individual; and (ii) administering MSCs to the individual so that the MSCs contact the depot of tropoelastin, thereby forming a cell of mesodermal lineage from an MSC.

Further aspects of the aspects described in the preceding paragraphs will become apparent from the following description, given by way of example and with reference to the accompanying drawings.

### BRIEF DESCRIPTION OF THE DRAWINGS

FIGS. 1A-1B. MSC proliferation on bare or tropoelastin-coated tissue culture plates (TCP), in media containing 10% (v/v) fetal bovine serum (FBS) (FIG. 1A) or 7% (v/v) FBS (FIG. 1B), with and without insulin-like growth factor-1 (IGF-1) and/or basic fibroblast growth factor (bFGF) growth factors. Panels show relative net cell increase at various days post-seeding. Asterisks directly above the columns represent statistical differences between bare and tropoelastin-coated TCP in each media formulation. As shown in FIGS. 1A and 1B, the bars on the graph alternate from left to right as: bare TCP and then TCP coated with tropoelastin (TE). *P<0.05; **P<0.01; ***P<0.001; ns, not significant.

FIGS. 2A-2B. MSC proliferation in decreasing amounts of serum. Cells were grown on bare, tropoelastin (TE)-coated or fibronectin (FN)-coated TCP in normal media (FIG. 2A). As shown in FIG. 2A, the bars on the graph alternate from left to right as: bare TCP, TCP coated with TE and TCP coated with FN. Cells were cultured on tropoelastin-coated TCP in normal media, on TCP in media containing IGF-1 and bFGF growth factors (GFs), or on tropoelastin-coated TCP in media supplemented with GFs (FIG. 2B). As shown in FIG. 2B, the bars on the graph alternate from left to right as: TE coated TCP, on TCP with media containing IGF-1 and bFGF growth factors and TE-coated TCP in media supplemented with GFs. Panels show the relative net cell increase at 3, 5 and 7 days post-seeding, normalized to the initial cell numbers at day 1. Asterisks directly above the columns represent statistical comparison with cells on tropoelastin-coated TCP in normal media. *P<0.05; **P<0.01; ***P<0.001.

FIGS. 3A-3C. MSC proliferation in media with tropoelastin in solution. Cells were grown on TCP in media supplemented with increasing concentrations of soluble tropoelastin (TE), or on TE-coated TCP in normal media (FIG. 3A). Panels show relative net cell increase at 3, 5 and 7 days post-seeding. Asterisks above individual columns depict statistical differences from the no-tropoelastin control. Cells were cultured on TCP in normal media, or in media supplemented with tropoelastin or growth factor/s (FIG. 3B). Panels show relative net cell increase at 3, 5 and 7 days post-seeding. Asterisks directly above the data columns indicate statistical differences from the normal media control. Cell proliferation for 7 days in normal media, or in media supplemented with κ-elastin (κELN), α-elastin (αELN), or tropoelastin (FIG. 3C). Asterisks indicate statistical differences from the normal media control. Cells were grown for up to 7 days in normal media, or media supplemented with fibronectin or tropoelastin in solution (FIG. 3D). Asterisks denote statistical differences from the normal media control. *P<0.05; **P<0.01; ***P<0.001; ns, not significant.

FIGS. 4A-4I. MSC attachment and spreading on tropoelastin. Cell adhesion to substrate-bound tropoelastin in the presence of EDTA (FIG. 4A). Cell binding to tropoelastin in cation-free buffer with increasing doses of exogenous Mg²⁺, Ca²⁺ and Mn²⁺ divalent cations (FIG. 4B). Cell spreading on tropoelastin with increasing concentrations of an anti-αvβ5 (FIG. 4C), anti-αvβ3 (FIG. 4D), or pan anti-αv integrin antibody (FIG. 4E). Cell spreading on fibronectin with and without the anti-αv integrin antibody is shown as a control. Cell spreading on tropoelastin in the presence of optimal inhibitory concentrations of anti-αvβ3, anti-αvβ5, combined anti-αvβ3 and anti-αvβ5, and anti-αv integrin antibodies (FIG. 4F). Cell spreading on TCP, and that on tropoelastin in the absence of antibodies or with a non-specific mouse IgG antibody, are also included as controls. Asterisks above the data columns refer to statistical differences from the no antibody control. Representative images of MSC spreading on tropoelastin, with and without integrin blocking antibodies (FIG. 4G). Confocal microscope images of MSCs adhered on tropoelastin- or BSA-coated TCP, stained for focal adhesion vinculin (green) and cell nuclei (blue) (FIG. 4H). The relative density of focal adhesion staining per cell is indicated. Scale bar: 20 μm. MSC proliferation after 7 days in the presence of an FAK inhibitor (FAK inhibitor 14 or a PKB/AKT inhibitor (perifosine). Cell numbers were normalized against uninhibited samples. Asterisks above individual columns represent comparison with the no-inhibitor control (FIG. 4I). *P<0.05; **P<0.01; ***P<0.001; ns, not significant.

FIGS. 5A-5B. MSC proliferation in the presence of fibroblast growth factor receptor (FGFR) (FIG. 5A) and integrin inhibitors (FIG. 5B). Cells were grown on TCP in normal media, in media with 20 μg/mL tropoelastin, or in bFGF-supplemented media for 7 days. Increasing doses of the FGFR inhibitor, SU-5402, were added to the media during the proliferation period (FIG. 5A). Cell numbers at each day were normalized against samples without SU-5402. Cell numbers in media containing tropoelastin or bFGF were compared with those in normal media at each inhibitor concentration to account for the non-specific toxicity of SU-5402. Optimal inhibitory concentrations of anti-αvβ3, anti-αvβ5, anti-αvβ5 and anti-αvβ3, or anti-αv, were added to the media over 7 days (FIG. 5B). As shown in FIG. 5B, the bars on the graph alternate from left to right as: normal media, media with TE, and media with bFGF. Controls without antibodies or with an antibody against a non-expressed integrin (anti-(38) were included. Green arrows above the bars of the bar graph, indicate cells grown in the presence of tropoelastin and αv integrin subunit antibodies. Asterisks above individual columns denote significant differences from cells in normal media at each antibody condition.

FIGS. 6A-6G. Migration of MSCs towards tropoelastin. Image showing the set-up of the migration assay (FIG. 6A). Cells were seeded in the middle chamber equidistant from flanking chambers containing substrate-bound tropoelastin or PBS. The well surface was divided into labelled regions within which cell numbers were measured as an indication of positional cell migration. Binary images of the labelled regions over 5 days, showing the spread of cell migration (FIG. 6B). Each black dot represents one cell nucleus as visualized under fluorescence microscopy. Comparative cell abundance within the regions that are adjacent to the areas coated with tropoelastin or PBS (FIG. 6C). Comparative cell abundance within the regions coated with tropoelastin or PBS (FIG. 6D). Total cell abundance within all regions over the experimental period (FIG. 6E). Cell migration towards increasing concentrations of tropoelastin as a diffusible chemoattractant in the bottom chamber of a Boyden chamber assay (FIG. 6F). Cells were incubated with or without 5 μg/mL anti-αv integrin antibody in the top chamber. Cell migration was normalized to the level of unstimulated migration exhibited by no tropoelastin controls. Asterisks above data points represent significant differences from the no-tropoelastin control. Cell chemotaxis to normal or tropoelastin-supplemented media in the presence of integrin-blocking antibodies (FIG. 6G). Controls without antibodies or with an antibody against a non-expressed integrin (anti-β8) were included. As shown in FIG. 6G, the bars on the graph alternate from left to right as: no antibody, with anti-αv, with anti-αvβ3, with anti-αvβ5, with anti-αvβ3/αvβ5 and with anti-αvβ8. Asterisks represent significant differences from the no-antibody control. *P<0.05; **P<0.01; ***P<0.001; ns, not significant; RFU, relative fluorescence unit.

FIG. 7. Model of tropoelastin modulation of MSC behavior. Substrate-bound or soluble tropoelastin attracts MSCs to migrate towards it. MSCs adhere and spread to the tropoelastin substrate, which triggers rapid cell expansion while simultaneously preserving MSC surface marker expression and tri-lineage differentiation potential. Unlike majority of anchorage-dependent matrix proteins, tropoelastin in its soluble form likewise promotes MSC proliferation and phenotypic maintenance. These signals from tropoelastin are conveyed via cell-surface integrin receptors, specifically αvβ3 and αvβ5, to induce potent motogenic and mitogenic MSC responses that mirror those to soluble growth factors such as bFGF.

FIGS. 8A-F. Effect of tropoelastin on MSC osteogenesis (FIGS. 8A-8B), adipogenesis (FIGS. 8C-8D), and chondrogenesis (FIGS. 8E-8F). Cells were expanded with or without tropoelastin, then transferred to inducing or non-inducing media with or without tropoelastin. In the adipogenesis experiment, cells were expanded without tropoelastin, expanded with tropoelastin until confluence at which point tropoelastin was removed during the post-confluence period, or expanded with tropoelastin until post-confluence prior to induction. As shown in FIG. 8A the bars on the graph alternate from left to right as: not induced without TE, not induced with TE, induced without TE, and induced with TE. As shown in FIG. 8C, the bars on the graph alternate from left to right as: non-induced, induced without TE, and induced with TE. As shown in FIG. 8E, the bars on the graph alternate from left to right as: not induced without TE, not induced with TE, induced without TE, and induced with TE.

FIGS. 9A-9F. Dose response to tropoelastin during MSC osteogenesis (FIGS. 9A-9B), adipogenesis (FIGS. 9C-9D) and chondrogenesis (FIGS. 9E-9F). Cells were expanded without or with tropoelastin at 2 μg/mL or 20 μg/mL, then transferred to inducing or non-inducing media with or without tropoelastin at 2 μg/mL or 20 μg/mL. In the adipogenesis experiment, a population of cells expanded in tropoelastin were cultured to post-confluence without tropoelastin prior to induction. As shown in FIG. 9A, the bars on the graph alternate from left to right as: not induced, induced, induced with 2 μg/mL TE, and induced with 20 μg/mL TE. As shown in FIG. 9C, the bars on the graph alternate from left to right as: not induced, induced, induced with 2 μg/mL TE, and induced with 20 μg/mL TE. As shown in FIG. 9E, the bars on the graph alternate from left to right as: not induced, induced, induced with 2 μg/mL TE, and induced with 20 μg/mL TE.

FIGS. 10A-D. Duration of the cells' tropoelastin memory during MSC osteogenesis (FIGS. 10A-10B) and chondrogenesis (FIGS. 10C-10D). Cells were expanded without tropoelastin, or with tropoelastin during days 2 to 5, 3 to 6, or 4 to 7 of the 7-day proliferation period, then transferred to differentiation media with or without tropoelastin.

FIGS. 11A-11F. Integrin inhibition of the tropoelastin effects on MSC osteogenesis. Inhibition of TE memory (FIG. 11A). As shown in FIG. 11A, the bars on the graph alternate from left to right as: without TE, without TE with anti-αv, without TE with anti-a5, without TE with anti-αv/a5, plus TE, TE with anti-αv, TE with anti-a5, and TE with anti-αv/a5. Expansion without TE (FIG. 11B). Expansion with TE (FIG. 11C). Expansion with TE plus anti-αv (FIG. 11D). Expansion with TE and anti-a5 (FIG. 11E). Expansion with TE with anti-αv/a5 (FIG. 11F). As shown in FIGS. 11B to 11F, the bars on the graph alternate from left to right as: induced without TE and induced with TE.

FIGS. 12A-12B. Effects of tropoelastin and hyaluronic acid on MSC osteogenesis. Bar graph demonstrating effect of tropoelastin and hyaluronic acid on MSC. (FIG. 12A). (FIG. 12B). Three different molecular weights of hyaluronic acid (30-50, 90-110, and 300-500 kDa) at concentrations of up to 500 μg/mL did not increase MSC osteogenesis over the tissue culture plastic control. The addition of tropoelastin to each of the hyaluronic acid formulations promoted MSC osteogenesis. These results indicate that tropoelastin is the primary pro-osteogenic agent in tropoelastin-hyaluronic acid composite materials.

FIGS. 13A-13C. Detection of surface-bound tropoelastin with an enzyme-linked immunosorbent assay (FIG. 13A). Tropoelastin (TE) was added to bare TCP, TCP pre-incubated with increasing concentrations of bovine serum albumin (BSA), or TCP pre-incubated with normal serum-containing media. In FIG. 13A from left to right, the bars on the graph are represented in the order of the samples in the key that is listed from top to bottom. Samples incubated with BSA or media, but without added tropoelastin, were used as negative controls. MSC proliferation on TCP in media containing increasing amounts of tropoelastin in solution, or on tropoelastin-coated TCP in normal media over 7 days (FIG. 13B). Panels show relative net cell increase at 3, 5 and 7 days post-seeding. Asterisks directly above data columns indicate statistical differences from the no-tropoelastin controls. MSC proliferation on TCP in normal media with and without soluble tropoelastin over 7 days (FIG. 13C). Tropoelastin was added at 20 μg/mL in solution, either on the day of seeding (D0), or at 3 days post-seeding (D3). Cell abundance in tropoelastin-supplemented media increases above that in normal media, corresponding to the time of tropoelastin addition.

FIGS. 14A-14B. Surface marker expression of MSCs expanded with tropoelastin. Cells were cultured on bare or tropoelastin-coated TCP in normal (10% (v/v) FBS) or reduced serum (6% (v/v) FBS) media, with or without IGF-1 and/or bFGF growth factors (FIG. 14A); or on TCP in normal media or in media containing 20 μg/mL soluble tropoelastin (TE) (FIG. 14B). (i) Percentage of the cell population expressing the positive MSC markers CD90, CD105, CD73, and the cocktail of negative markers CD34 CD45, CD11b, CD79a and HLA-DR after 5 or 7 days culture. Marker expression was quantified as the percentage of positive events detected from gated singlet viable cells. (ii) Representative flow cytometry dot plots of cells grown in various culture conditions at 7 days post-seeding. The first row depicts the selection gating for cells that do not express the negative markers. The second row shows the population of lineage-negative cells which express both positive markers CD90 and CD105. The third row shows the population of CD90+ and CD105+ cells which also express the MSC marker CD73. Cells stained with isotype antibody controls for all markers are also shown.

FIG. 15. Tri-lineage differentiation of MSCs expanded with tropoelastin. Cells were cultured on TCP or tropoelastin (TE)-coated TCP, in normal media (NM) or reduced serum media (RSM) supplemented with tropoelastin or IGF-1 and bFGF growth factors for 7 days, then harvested and differentiated into adipogenic, osteogenic, and chondrogenic lineages. Induced and non-induced cells were stained for intracellular lipid droplets with Oil Red O, mineralized calcium nodules with Alizarin Red, and glycosaminoglycans with Alcian Blue. Scale bar: 50 μm.

FIGS. 16A-16B. Chemotactic behavior of MSCs. Cell migration towards increasing concentrations of tropoelastin as a diffusible chemoattractant in the bottom chamber of a Boyden chamber assay (FIG. 16A). Cells were incubated with or without 5 μg/mL anti-β8 integrin antibody in the top chamber. Cell migration was normalized to the level of unstimulated migration exhibited by no tropoelastin controls. Cell chemotaxis to normal or growth factor-supplemented media in the presence of integrin-blocking antibodies (FIG. 16B). Controls without antibodies or with an antibody against a non-expressed integrin (anti-β8) were included. As shown in FIG. 16B, the bars on the graph alternate from left to right as: no antibody, plus anti-αv, plus anti-age, plus anti-αvβ5, and plus anti-β8.

FIGS. 17A-17B. MSC abundance in the presence of (A) fibroblast growth factor receptor (FGFR) and (B) integrin inhibitors, one day post-seeding. Cells were grown on TCP in normal media, in media with 20 μg/mL tropoelastin (TE), or in bFGF-supplemented media. (I) Increasing doses of the FGFR inhibitor, SU-5402, were added to the media. Cell numbers were normalized against samples without SU-5402. Cell numbers in media containing tropoelastin or bFGF were compared with those in normal media at each inhibitor concentration to account for the non-specific toxicity of SU-5402. (B) Optimal inhibitory concentrations of anti-αvβ3, anti-αvβ5, anti-αvβ5 and anti-αvβ3, or anti-αv, were added to the media. Controls without antibodies or with an antibody against a non-expressed integrin (anti-β8) were included. Green arrows indicate cells grown in the presence of tropoelastin and αv integrin subunit antibodies. Asterisks above individual columns denote significant differences from cells in normal media at each antibody condition.

### DETAILED DESCRIPTION

As described in the embodiments herein, unknown properties of tropoelastin have been identified as well as methods for use of tropoelastin in cell culture of MSCs based on those properties.

As used herein, except where the context requires otherwise, the term ‘comprise’ and variations of the term, such as ‘comprising’, ‘comprises’ and ‘comprised’, are not intended to exclude further additives, components, integers or steps.

The term ‘expansion phase’ or ‘proliferation phase’ refers to a cell culture process whereby MSCs are cultured with tropoelastin, with or without other proliferation factors for inducing production of MSCs. Differentiated cells are not produced at the completion of this phase. The only cells remaining are those that are MSCs i.e. having MSC phenotype and/or plasticity.

The term ‘expansion’ or ‘cell expansion’ refers to methods to increase a number of cells so that the cells are expanded in vitro before use, such as clinical use. As described in some embodiments, cells may be expanded in a tissue culture vessel or plate in the presence of a culture medium. Without being limiting, the culture medium may be supplemented with growth factors, serum and specific additives. In some embodiments herein, the cells are expanded in the presence of tropoelastin.

In some embodiments, the cells are expanded in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the cells are expanded in the absence of in the absence of IGF-1 and bFGF.

The term ‘differentiation phase’ refers to a cell culture process whereby MSCs are cultured with factors for inducing production of differentiated cells.

The term ‘differentiation’ or ‘cellular differentiation’ refers to a process in which a cell changes to a specialized type of a cell. In some cases, a cell may change in size, shape and in response to outside signalling.

In some embodiments herein, cells are differentiated in the presence of tropoelastin and differentiation factors. In some embodiments, the presence of tropoelastin increases the efficacy of cell differentiation.

The term “Tropoelastin” refers to a monomeric protein from which elastin is formed. Tropoelastin is generally not cross-linked, covalently or otherwise. Tropoelastin may reversibly coacervate. Thus, tropoelastin is distinguished from elastin because elastin consists of covalently cross linked tropoelastin which cannot reversibly coacervate. Tropoelastin may be synthetic, for example it may be derived from recombinant expression or other synthesis, or it may be obtained from a natural source such as porcine aorta. As generally known in the art, tropoelastin may exist in the form of a variety of fragments.

The term ‘Cells of mesodermal lineage’ refers to cells derived from MSC differentiation, including determined cells such as osteocytes, adipocytes and chondrocytes and the precursors of determined cells. ‘Cells of mesodermal lineage’ does not refer to an MSC.

The term ‘Mesenchymal stem cells (MSCs)’ refers to multipotent adult stem cells. Without being limiting, these cells may be found from multiple tissue sources, such as umbilical cord, bone marrow and fat tissue. MSC are nonhematopoietic stromal cells that may self-renew by dividing and are capable of differentiating into multiple types of tissues such as bone (osteoblasts), cartilage (chondrocytes), muscle (myocytes), fat cells (adipocytes), and connective tissue, for example. Without being limiting, MSC may be identified by the expression of CD105(SH2) and CD73 (SH3/4). Without being limiting, the cells may be negative for hematopoietic markers, such as CD34, CD45 and CD14, for example.

The term ‘Bone marrow cells’ refers to the semi-solid tissue that is found within the spongy center or cancellous sites of bones. This tissue is comprised of hematopoietic cells, marrow adipose tissue, and supportive stromal cells, for example. In some embodiments described herein, are methods of expansion and differentiation of bone marrow cells.

‘Stem cell plasticity’ or ‘transdifferentiation’ refers to the ability of a cell to give rise to a cell type that is considered to be outside their normal repertoire of differentiation for the location where they are found. It can also be considered as the capacity of a cell to convert to cells of other types of tissue.

‘Partially dissolve’ or ‘partially soluble’ refers to the description of a solute that will dissolve as a small concentration within a solvent, but will not dissolve completely above a certain concentration. Partial dissolution for tropoelastin may be described as firstly, the concentration-dependent solubility of tropoelastin in solvent which is limited to below 300 mg/mL. Concentrations that are below this amount may be used. Secondly the dissolution of tropoelastin is the fraction of tropoelastin that has dissolved off a surface or another depot, where some tropoelastin has not yet been dissolved.

Identified properties of tropoelastin that suggest tropoelastin to be a useful candidate for commercial production of cells of mesodermal lineage, especially osteocytes, chondrocytes and adipocytes, and in particular in ex vivo or in vivo production of cells, especially autologous cells. The differentiation yield of some cells may be enhanced in circumstances where tropoelastin is utilised to drive or induce proliferation of MSCs either prior to, or during a differentiation phase.

In more detail, it has been found that tropoelastin provides for a higher yield of osteocytes, chondrocytes and adipocytes where the tropoelastin is used in an expansion phase to induce proliferation of MSCs and in a differentiation phase involving osteogenic, chondrogenic or adipogenic differentiation of those MSCs. Therefore, according to the disclosure, with respect to osteocyte, chondrocyte or adipocyte production, tropoelastin may be used during an expansion phase and a differentiation phase.

In one embodiment, a method for forming cells of mesodermal lineage from MSCs is provided. The method comprises contacting MSCs with (i) at least one differentiation factor for inducing formation of cells of mesodermal lineage from MSCs; and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

The tropoelastin may be arranged on a cell culture surface of a cell culture vessel to enable the MSCs to contact the tropoelastin when the MSCs are contacted with the cell culture surface.

The tropoelastin may be partially or fully solubilized in a cell culture medium for culture of an MSC.

The method may include the following steps: (i) contacting MSCs with at least one factor for inducing proliferation of MSCs, thereby forming a population of MSCs; and (ii) contacting the population of MSCs with at least one differentiation factor for inducing formation of cells of mesodermal lineage from MSC and tropoelastin. The method comprises (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, step (i) is performed in the absence of in the absence of IGF-1 and bFGF. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

In a particularly preferred embodiment, a method for forming osteocytes from MSCs is provided. The method comprises: (i) contacting MSCs with tropoelastin during an expansion phase, to induce proliferation of MSCs, thereby forming an expanded population of MSCs; and (ii) contacting the expanded population of MSCs with tropoelastin during a differentiation phase. Preferably the expansion phase includes the use of at least one factor for inducing expansion or proliferation of MSCs. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the step (i) is performed in the absence of in the absence of IGF-1 and bFGF. Preferably the differentiation phase includes use of factors for inducing formation of osteocytes from MSCs. These include dexamethasone, ascorbate, beta-glycerophosphate. In some embodiments, the at least one factor comprises dexamethasone, ascorbate and/or beta-glycerophosphate. More preferably, the expansion phase is completed independently of the differentiation phase.

In some embodiments, the number of cells of mesodermal lineage formed from MSC in the presence of tropoelastin during an expansion phase is greater than the number of cells of mesodermal lineage formed in the absence of tropoelastin during the expansion phase. In some embodiments, tropoelastin promotes stem cell expansion and recruitment.

In a particularly preferred embodiment, a method for forming adipocytes from MSCs is provided. The method comprises: (i) contacting MSCs with tropoelastin during an expansion phase, to induce proliferation of MSCs, thereby forming an expanded population of MSCs; and (ii) contacting the expanded population of MSCs with tropoelastin during a differentiation phase. Preferably the expansion phase includes the use of at least one factor for inducing expansion or proliferation of MSCs. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the step (i) is performed in the absence of in the absence of IGF-1 and bFGF. Preferably the differentiation phase includes use of factors for inducing formation of adipocytes from MSCs. These include h-insulin, dexamethasone, indomethacin, 3-isobutyl-1-methylxanthine. In some embodiments, the at least one factor comprises h-insulin, dexamethasone, indomethacin and/or 3-isobutyl-1-methylxanthine. More preferably the expansion phase is completed independently of the differentiation phase. In some embodiments, the number of cells of mesodermal lineage formed from MSC in the presence of tropoelastin during an expansion phase is greater than the number of cells of mesodermal lineage formed in the absence of tropoelastin during the expansion phase. In some embodiments, tropoelastin promotes stem cell expansion and recruitment.

In a particularly preferred embodiment, a method for forming chondrocytes from MSCs is provided. The method comprises: (i) contacting MSCs with tropoelastin during an expansion phase, to induce proliferation of MSCs, thereby forming an expanded population of MSCs; and (ii) contacting the expanded population of MSCs during a differentiation phase with at least one factor for inducing formation of chondrocytes from MSCs. Preferably the expansion phase includes the use of factors for inducing expansion or proliferation of MSCs. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the step (i) is performed in the absence of in the absence of IGF-1 and bFGF. Preferably the differentiation phase includes use of factors for inducing formation of chondrocytes from MSCs. These include dexamethasone, ascorbate, insulin-transferrin-selenium, sodium pyruvate, proline. In some embodiments, the at least one factor comprises dexamethasone, ascorbate, insulin-transferrin-selenium, sodium pyruvate and/or proline. More preferably the expansion phase is completed independently of the differentiation phase.

It will be understood that in the above described methods, the tropoelastin is not provided with silk protein. In some embodiments, the number of cells of mesodermal lineage formed from MSC in the presence of tropoelastin during an expansion phase is greater than the number of cells of mesodermal lineage formed in the absence of tropoelastin during the expansion phase. In some embodiments, tropoelastin promotes stem cell expansion and recruitment. Tropoelastin may preserve the ability of the cells to develop into different types of cells.

The tropoelastin may be provided in the form of a complex with hyaluronic acid that is partially or completely soluble, wherein the tropoelastin monomers are linked together by hyaluronic acid. In some embodiments, the tropoelastin is cross-linked to the hyaluronic acid.

The cell of mesodermal lineage produced by the method may be an osteocyte, chondrocyte or adipocyte.

In some embodiments, the MSCs are human MSCs.

In another embodiment, a composition of cells formed from a method described above is provided.

The composition may be a substantially pure form of osteocytes.

The composition may include tropoelastin and/or hyaluronic acid.

In another embodiment, a method for treating an individual having a bone disorder or fracture is provided. The method comprises providing a composition described above to the individual is provided, thereby treating the individual for a bone disorder or fracture. The composition may include tropoelastin and an MSC. The composition may additionally include one or more factors for differentiation of an MSC to form an osteocyte or precursor of an osteocyte. In some embodiments, the composition is administered to the individual at a local site, wherein the local site is an area of the bone disorder or fracture.

In another embodiment, a method for treating an individual having a region of fat loss or atrophy arising from a disease or trauma, or an individual requiring surgical enhancement arising from surgery or disease, is provided. The method includes providing a composition described above to the individual, thereby treating the individual. The composition may include tropoelastin and an MSC. The composition may additionally include one or more factors for differentiation of an MSC to form an adipocyte or precursor of an adipocyte. In some embodiments, the composition is administered to the individual at a local site, wherein the local site is an area of the fat loss or atrophy.

In another embodiment, a method for treating an individual having a cartilage disorder including providing a composition described above to the individual, is provided, thereby treating the individual for a cartilage disorder. The composition may include tropoelastin and an MSC. The composition may additionally include one or more factors for differentiation of an MSC to form a chondrocyte or precursor of a chondrocyte. In some embodiments, the composition is administered to the individual at a local site, wherein the local site is an area of the cartilage disorder.

As described in the embodiments herein, properties of tropoelastin have been identified, that suggest tropoelastin to be a useful candidate for commercial production of MS Cs. In particular, it has been found that tropoelastin, whether provided in a form bound to a solid phase, or provided in solution, is able to induce proliferation of MSCs in a manner that retains the stemness or plasticity inherent in MSCs. “Stemness” has its plain and ordinary meaning and may refer to essential characteristic of a stem cell that distinguishes it from ordinary cells. In some embodiments, wherein the tropoelastin is added to a solution, the tropoelastin is prevented from adhesion to a solid phase, wherein the solid phase is a vehicle for holding the cells. This can be achieved in a medium containing a substantially reduced serum component, and in the absence of certain factors, such as IGF-1 and bFGF, that are normally utilised for MSC proliferation and production. Without wanting to be bound by hypothesis, it appears that the mechanism of action, at least insofar as MSC attachment and spreading is concerned, requires direct engagement or interaction as between tropoelastin and the cell surface. In more detail, the interaction or engagement is understood to be between tropoelastin and αvβ5 and αvβ3 molecules on MSCs, and the mechanism is ablated where MSCs are sterically hindered or blocked from contact with tropoelastin. In some embodiments, wherein the tropoelastin is added to a solution, the tropoelastin is prevented from adhesion to a solid substrate, wherein the solid substrate is a vehicle for holding the cells.

In one embodiment, a method for inducing proliferation of MSCs, is provided. The method comprises contacting MSCs with tropoelastin to induce proliferation of MSCs, wherein the number of MSCs formed in the presence of tropoelastin is greater than the number of MSCs formed in the absence of tropoelastin, thereby inducing proliferation of MSCs.

The tropoelastin may be arranged on a cell culture surface of a cell culture vessel to enable the MSCs to contact the tropoelastin when the MSCs are contacted with the cell culture surface. Preferably the tropoelastin is arranged to enable MSCs to bind to tropoelastin via αvβ5 and αvβ3 molecules located on the MSC plasma membrane.

The tropoelastin may be partially or fully solubilized in a cell culture medium for culture of an MSC.

The method comprises culturing MSCs in a first medium containing tropoelastin to form a tropoelastin-cultured MSC population, thereafter, culturing said tropoelastin-cultured MSC population in a second medium wherein the second medium comprises a factor for inducing proliferation of an MSC.

In one embodiment, the expansion phase is performed in the presence of tropoelastin and in the absence of a factor for inducing expansion or proliferation of an MSC, especially in the absence of IGF1 and or bFGF.

In one embodiment, the expansion phase is performed in the presence of tropoelastin and in the absence of a protein source, such as serum.

It will be understood that in the above described methods, the tropoelastin is not provided with silk protein.

The tropoelastin may be provided in the form of a complex with hyaluronic acid that is partially or completely soluble, wherein the tropoelastin monomers are linked together by hyaluronic acid.

In some embodiments, the tropoelastin is cross-linked to the hyaluronic acid.

In some embodiments, the MSCs are human MSCs.

In another embodiment, a composition of MSCs formed from a method described above, is provided.

The composition may be a substantially pure form of MSCs.

The composition may include tropoelastin and/or hyaluronic acid.

As described in the embodiments herein, a use of tropoelastin in expansion and differentiation of MSCs in vitro has been established. Tropoelastin is established as a factor that is mitogenic for MSCs, enabling MSC proliferation in the absence of other proliferative factors, enabling a greater production of MSCs in the presence of other proliferative factors and absence of tropoelastin. Tropoelastin is also established as a factor that results in the increased expansion of mesodermal precursors and determined cells when provided with mesenchymal differentiation factors, providing for increased numbers of precursor and determined cells. As factors for differentiation of MSCs to form cells of mesodermal lineage are naturally found in mammalian tissue, it follows that ex vivo obtained tropoelastin/MSC compositions can be used to create cells of mesodermal lineage in vivo. Three applications are foreshadowed: (i) where MSCs harvested during surgery or biopsy are contacted with tropoelastin and more or less immediately administered to an individual at a tissue site where cells of mesodermal lineage are required; (ii) where MSCs harvested during surgery or biopsy are ex vivo contacted with tropoelastin for a time period to enable expansion of MSC cell number and then the composition which includes expanded MSCs and tropoelastin is administered to an individual at a tissue site where cells of mesodermal lineage are required; and (iii) where MSCs harvested during surgery or biopsy are ex vivo contacted with tropoelastin for a time period to enable expansion of MSC cell number and then the composition is contacted with one or more factors for inducing differentiation and then administered to an individual at a tissue site where cells of mesodermal lineage are required.

Thus, in a further embodiment, a method for forming a cell of mesodermal lineage from an MSC in an individual, is provided. The method comprises (i) contacting MSCs with tropoelastin to form a composition of MSCs and tropoelastin and (ii) administering the composition to an individual requiring formation of cells of mesodermal lineage from an MSC, thereby forming a cell of mesodermal lineage from an MSC. In some embodiments, the composition is administered to the individual at a localized site.

According to this embodiment, it is the endogenous differentiation factors of the individual which provide for differentiation of the MSCs when administered to the individual.

According to this embodiment, the MSCs may be contacted with tropoelastin and administered to the individual within hours, for example 1 to 6 hours, preferably less than 1 hour after isolation from the individual, so that for example the steps of isolation of MSCs, contact with tropoelastin, and administration to the individual are accomplished within a single surgical procedure.

In another embodiment, a method for forming a cell of mesodermal lineage from an MSC is provided. The method comprises (i) contacting MSCs with tropoelastin to induce proliferation of MSCs, thereby forming a composition of MSCs and tropoelastin; and thereafter, (ii) administering the composition to an individual requiring formation of cells of mesodermal lineage from an MSC, thereby forming a cell of mesodermal lineage from an MSC. According to this embodiment, it is the endogenous differentiation factors of the individual which provide for differentiation of the MSCs when administered to the individual.

In the above described embodiments, the tropoelastin and MSC are generally administered in the form of a composition containing both tropoelastin and MSC. In these embodiments, the composition may include additional factors for proliferation or differentiation of MSCs, or these additional factors may be administered to the individual separately. In some embodiments, the proliferation factor comprises tropoelastin. In some embodiments, the proliferation factor comprises serum. In some embodiments, the differentiation factor(s) comprises dexamethasone, ascorbate and/or beta glycerophosphate. In some embodiments, the differentiation factor(s) comprises h-insulin, dexamethasone, indomethacin and/or 3-isobutyl-1-methyl-xanthine. In some embodiments, the differentiation factor(s) comprises dexamethasone, ascorbate, insulin-transferrin-selenium, sodium pyruvate and/or proline.

In another embodiment, a method for forming a cell of mesodermal lineage from an MSC is provided. The method comprises (i) administering tropoelastin to an individual requiring formation of cells of mesodermal lineage from an MSC thereby forming a depot of tropoelastin in the individual; and (ii) administering MSCs to the individual so that the MSCs contact the depot of tropoelastin, thereby forming a cell of mesodermal lineage from an MSC. One advantage of the method is that it avoids the step of contacting tropoelastin and MSC prior to administration to the individual. Specifically, the individual can be administered tropoelastin at a site requiring production of MSC or mesodermal cells derived from same prior to the isolation of MSCs from the individual. In some embodiments, the tropoelastin is administered to the individual at a local site, wherein the local site is an area of the bone disorder or fracture. In some embodiments, the tropoelastin is administered to the individual at a local site, wherein the local site is an area of the fat loss or atrophy. In some embodiments, the tropoelastin is administered to the individual at a local site, wherein the local site is an area of the cartilage disorder. After isolation, the MSCs are simply injected into the site where tropoelastin has been prior established. This enables the formation of mesodermal cells from the MSCs.

In the above described embodiments, the MSCs are generally harvested from an individual by established techniques.

Typically, the MSCs are autologous.

In the above described embodiments, the number of cells utilised in the expansion phase is generally about 10³ to 10⁵ cells.

MSCs for use in the embodiments described herein can be derived from a variety of sources, including but not limited to bone marrow, cord cells, adipose tissue, molar cells, amniotic fluid and peripheral blood, for example. In some embodiments, the MSC is derived from bone marrow, cord cells, adipose tissue, molar cells, amniotic fluid and peripheral blood.

In culture, MSCs express CD73, CD90 and CD105. They may lack CD11b, CD14, CD19, CD34, CD45, CD79a and HLA DR.

In the above described embodiments, the concentration of tropoelastin may generally range from about 0.01 μg/ml to about 200 mg/ml, more preferably from about 1 μg/ml to about 100 mg/ml, more preferably from about 1 μg/ml to about 75 mg/ml, from about 1 μg/ml to about 50 mg/ml, or from about 10 μg/ml to about 100 mg/ml, from about 10 μg/ml to about 75 mg/ml, from about 10 μg/ml to about 50 mg/ml.

Tropoelastin may be obtained by purification from a suitable source (e.g. from humans or other animals) or produced by standard recombinant DNA techniques such as is described in, for example, Maniatis.

Recombinant tropoelastin may incorporate modifications (e.g. amino acid substitutions, deletions, and additions of heterologous amino acid sequences), thereby forming tropoelastin analogues which may, for example, enhance biological activity or expression of the respective protein. In some embodiments, the tropoelastin comprises a sequence set forth in SEQ ID NO: 1.

In a preferred embodiment, the methods utilise the SHELδ26A analogue (WO 1999/03886) for the various applications described herein including for proliferation and/or differentiation of MSCs. The amino acid sequence of SHELδ26A (SEQ ID NO: 1) is:

(SEQ ID NO: 1;
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPSVGGV
PGVGGVPGVGISPEAQAAAAAKAAKYGVGTPAAAAAKAAAKAAQFGLVPG
VGVAPGVGVAPGVGVAPGVGLAPGVGVAPGVGVAPGVGVAPGIGPGGVAA
AAKSAAKVAAKAQLRAAAGLGAGIPGLGVGVGVPGLGVGAGVPGLGVGAG
VPGFGAVPGALAAAKAAKYGAAVPGVLGGLGALGGVGIPGGVVGAGPAAA
AAAAKAAAKAAQFGLVGAAGLGGLGVGGLGVPGVGGLGGIPPAAAAKAAK
YGAAGLGGVLGGAGQFPLGGVAARPGFGLSPIFPGGACLGKACGRKRK)

In alternative embodiments, the tropoelastin isoform is the SHEL isoform (WO 1994/14958; included by reference in its entirety herein) (SEQ ID NO: 2; SMGGVPGAIPGGVPGGVFYPGAGLGALGGGALGPGGKPLKPVPGGLAGAGLGAGLGA FPAVTFPGALVPGGVADAAAAYKAAKAGAGLGGVPGVGGLGVSAGAVVPQPGAGVK PGKVPGVGLPGVYPGGVLPGARFPGVGVLPGVPTGAGVKPKAPGVGGAFAGIPGVGPF GGPQPGVPLGYPIKAPKLPGGYGLPYTTGKLPYGYGPGGVAGAAGKAGYPTGTGVGPQ AAAAAAAKAAAKFGAGAAGVLPGVGGAGVPGVPGAIPGIGGIAGVGTPAAAAAAAAA AKAAKYGAAAGLVPGGPGFGPGVVGVPGAGVPGVGVPGAGIPVVPGAGIPGAAVPGV VSPEAAAKAAAKAAKYGARPGVGVGGIPTYGVGAGGFPGFGVGVGGIPGVAGVPSVG GVPGVGGVPGVGISPEAQAAAAAKAAKYGVGTPAAAAAKAAAKAAQFGLVPGVGVA PGVGVAPGVGVAPGVGLAPGVGVAPGVGVAPGVGVAPGIGPGGVAAAAKSAAKVAA KAQLRAAAGLGAGIPGLGVGVGVPGLGVGAGVPGLGVGAGVPGFGAGADEGVRRSLS PELREGDPSSSQHLPSTPSSPRVPGALAAAKAAKYGAAVPGVLGGLGALGGVGIPGGVV GAGPAAAAAAAKAAAKAAQFGLVGAAGLGGLGVGGLGVPGVGGLGGIPPAAAAKAA KYGAAGLGGVLGGAGQFPLGGVAARPGFGLSPIFPGGACLGKACGRKRK) or a protease resistant derivative of the SHEL or SHELδ26A isoforms (WO 2000/04043; included by reference in its entirety herein). As described in WO 2000/04043, the protein sequences of tropoelastin described may have a mutated sequence that leads to a reduced or eliminated susceptibility to digestion by proteolysis. Without being limiting, the tropoelastin amino acid sequence has a reduced or eliminated susceptibility to serine proteases, thrombin, kallikrein, metalloproteases, gelatinase A, gelatinase B, serum proteins, trypsin or elastase, for example. In some embodiments, the tropoelastin comprises a sequence set forth in SEQ ID NO: 3 (SHELδ26A isoform) (SEQ ID NO: 3: GGVPGAIPGGVPGGVFYPGAGLGALGGGALGPGGKPLKPVPGGLAGAGLGAGLGAFPA VTFPGALVPGGVADAAAAYKAAKAGAGLGGVPGVGGLGVSAGAVVPQPGAGVKPGK VPGVGLPGVYPGGVLPGARFPGVGVLPGVPTGAGVKPKAPGVGGAFAGIPGVGPFGGP QPGVPLGYPIKAPKLPGGYGLPYTTGKLPYGYGPGGVAGAAGKAGYPTGTGVGPQAAA AAAAKAAAKFGAGAAGVLPGVGGAGVPGVPGAIPGIGGIAGVGTPAAAAAAAAAAKA AKYGAAAGLVPGGPGFGPGVVGVPGAGVPGVGVPGAGIPVVPGAGIPGAAVPGVVSPE AAAKAAAKAAKYGARPGVGVGGIPTYGVGAGGFPGFGVGVGGIPGVAGVPSVGGVPG VGGVPGVGISPEAQAAAAAKAAKYGVGTPAAAAAKAAAKAAQFGLVPGVGVAPGVG VAPGVGVAPGVGLAPGVGVAPGVGVAPGVGVAPGIGPGGVAAAAKSAAKVAAKAQL RAAAGLGAGIPGLGVGVGVPGLGVGAGVPGLGVGAGVPGFGAVPGALAAAKAAKYG AAVPGVLGGLGALGGVGIPGGVVGAGPAAAAAAAKAAAKAAQFGLVGAAGLGGLGV GGLGVPGVGGLGGIPPAAAAKAAKYGAAGLGGVLGGAGQFPLGGVAARPGFGLSPIFP GGACLGKACGRKRK) In some embodiments, the tropoelastin comprises a sequence set forth in SEQ ID NO: 4 (SHELδmod isoform) (SEQ ID NO: 4: GGVPGAVPGGVPGGVFYPGAGFGAVPGGVADAAAAYKAAKAGAGLGGVPGVGGLGV SAGAVVPQPGAGVKPGKVPGVGLPGVYPGFGAVPGARFPGVGVLPGVPTGAGVKPKA PGVGGAFAGIPGVGPFGGPQPGVPLGYPIKAPKLPGGYGLPYTTGKLPYGYGPGGVAGA AGKAGYPTGTGVGPQAAAAAAAKAAAKFGAGAAGFGAVPGVGGAGVPGVPGAIPGIG GIAGVGTPAAAAAAAAAAKAAKYGAAAGLVPGGPGFGPGVVGVPGFGAVPGVGVPG AGIPVVPGAGIPGAAGFGAVSPEAAAKAAAKAAKYGARPGVGVGGIPTYGVGAGGFPG FGVGVGGIPGVAGVPSVGGVPGVGGVPGVGISPEAQAAAAAKAAKYGVGTPAAAAAK AAAKAAQFGLVPGVGVAPGVGVAPGVGVAPGVGLAPGVGVAPGVGVAPGVGVAPGI GPGGVAAAAKSAAKVAAKAQLRAAAGLGAGIPGLGVGVGVPGLGVGAGVPGLGVGA GVPGFGAVPGALAAAKAAKYGAVPGVLGGLGALGGVGIPGGVVGAGPAAAAAAAKA AAKAAQFGLVGAAGLGGLGVGGLGVPGVGGLGGIPPAAAAKAAKYGAAGLGGVLGG AGQFPLGGVAARPGFGLSPIFPGGACLGKACGRKRK).

Tropoelastin analogues generally have a sequence that is homologous to human tropoelastin sequence. Percentage identity between a pair of sequences may be calculated by the algorithm implemented in the BESTFIT computer program. Another algorithm that calculates sequence divergence has been adapted for rapid database searching and implemented in the BLAST computer program. In comparison to the human sequence, the tropoelastin polypeptide sequence may be only about 60% identical at the amino acid level, about 70% or more identical, about 80% or more identical, about 90% or more identical, about 95% or more identical, about about 97% or more identical, or greater than about 99% identical.

Conservative amino acid substitutions (e.g., Glu/Asp, Val/lle, Ser/Thr, Arg/Lys, Gln/Asn) may also be considered when making comparisons because the chemical similarity of these pairs of amino acid residues are expected to result in functional equivalency in many cases. Amino acid substitutions that are expected to conserve the biological function of the polypeptide would conserve chemical attributes of the substituted amino acid residues such as hydrophobicity, hydrophilicity, side-chain charge, or size.

The codons used may also be adapted for translation in a heterologous host by adopting the codon preferences of the host. This would accommodate the translational machinery of the heterologous host without a substantial change in chemical structure of the polypeptide. The use of codon optimization has been previously described and can be appreciated for use in optimizing the levels of protein translated.

Recombinant forms of tropoelastin can be produced as shown in WO 1999/03886. These sequences are: SEQ ID NO: 5 (SEQ ID NO: 5; SMGGVPGAIPGGVPGGVFYPGAGLGALGGGALGPGGKPLKPVPGGLAGAGLGAGLGA FPAVTFPGALVPGGVADAAAAYKAAKAGAGLGGVPGVGGLGVSAGAVVPQPGAGVK PGKVPGVGLPGVYPGGVLPGARFPGVGVLPGVPTGAGVKPKAPGVGGAFAGIPGVGPF GGPQPGVPLGYPIKAPKLPGGYGLPYTTGKLPYGYGPGGVAGAAGKAGYPTGTGVGPQ AAAAAAAKAAAKFGAGAAGVLPGVGGAGVPGVPGAIPGIGGIAGVGTPAAAAAAAAA AKAAKYGAAAGLVPGGPGFGPGVVGVPGAGVPGVGVPGAGIPVVPGAGIPGAAVPGV VSPEAAAKAAAKAAKYGARPGVGVGGIPTYGVGAGGFPGFGVGVGGIPGVAGVPSVG GVPGVGGVPGVGISPEAQAAAAAKAAKYGVGTPAAAAAKAAAKAAQFGLVPGVGVA PGVGVAPGVGVAPGVGLAPGVGVAPGVGVAPGVGVAPGIGPGGVAAAAKSAAKVAA KAQLRAAAGLGAGIPGLGVGVGVPGLGVGAGVPGLGVGAGVPGFGAGADEGVRRSLS PELREGDPSSSQHLPSTPSSPRVPGALAAAKAAKYGAAVPGVLGGLGALGVGIPGGVVG AGPAAAAAAAKAAAKAAQFGLVGAAGLGGLGVGGLGVPGVGGLGGIPPAAAAKAAK YGAAGLGGVLGGAGQFPLGGVAARPGFGLSPIFPGGACLGKACGRKRK); SEQ ID NO: 6 (SEQ ID NO: 6; GGVPGAIPGGVPGGVFYPGAGLGALGGGALGPGGKPLKPVPGGLAGAGLGAGLGAFPA VTFPGALVPGGVADAAAAYKAAKAGAGLGGVPGVGGLGVSAGAVVPQPGAGVKPGK VPGVGLPGVYPGGVLPGARFPGVGVLPGVPTGAGVKPKAPGVGGAFAGIPGVGPFGGP QPGVPLGYPIKAPKLPGGYGLPYTTGKLPYGYGPGGVAGAAGKAGYPTGTGVGPQAAA AAAAKAAAKFGAGAAGVLPGVGGAGVPGVPGAIPGIGGIAGVGTPAAAAAAAAAAKA AKYGAAAGLVPGGPGFGPGVVGVPGAGVPGVGVPGAGIPVVPGAGIPGAAVPGVVSPE AAAKAAAKAAKYGARPGVGVGGIPTYGVGAGGFPGFGVGVGGIPGVAGVPSVGGVPG VGGVPGVGISPEAQAAAAAKAAKYGVGTPAAAAAKAAAKAAQFGLVPGVGVAPGVG VAPGVGVAPGVGLAPGVGVAPGVGVAPGVGVAPGIGPGGVAAAAKSAAKVAAKAQL RAAAGLGAGIPGLGVGVGVPGLGVGAGVPGLGVGAGVPGFGAVPGALAAAKAAKYG AAVPGVLGGLGALGGVGIPGGVVGAGPAAAAAAAKAAAKAAQFGLVGAAGLGGLGV GGLGVPGVGGLGGIPPAAAAKAAKYGAAGLGGVLGGAGQFPLGGVAARPGFGLSPIFP GGACLGKACGRKRK); SEQ ID NO: 7 (SEQ ID NO: 7; MGGVPGAVPGGVPGGVFYPGAGFGAVPGGVADAAAAYKAAKAGAGLGGVPGVGGL GVSAGAVVPQPGAGVKPGKVPGVGLPGVYPGFGAVPGARFPGVGVLPGVPTGAGVKP KAPGVGGAFAGIPGVGPFGGPQPGVPLGYPIKAPKLPGGYGLPYTTGKLPYGYGPGGVA AAGKAGYPTGTGVGPQAAAAAAAKAAAKFGAGAAGFGAVPGVGGAGVPGVPGAIPGI GGIAGVGTPAAAAAAAAAAKAAKYGAAAGLVPGGPGFGPGVVGVPGFGAVPGVGVP GAGIPVVPGAGIPGAAGFGAVSPEAAAKAAAKAAKYGARPGVGVGGIPTYGVGAGFFP GFGVGVGGIPGVAGVPSVGGVPGVGGVPGVGISPEAQAAAAAKAAKYGVGTPAAAAA KAAAKAAQFGLVPGVGVAPGVGVAPGVGVAPGVGLAPGVGVAPGVGVAPGVGVAPG IGPGGVAAAAKSAAKVAAKAQLRAAAGLGAGIPGLGVGVGVPGLGVGAGVPGLGVGA GVPGFGAVPGALAAAKAAKYGAVPGVLGGLGALGGVGIPGGVVGAGPAAAAAAAKA AAKAAQFGLVGAAGLGGLGVGGLGVPGVGGLGGIPPAAAAKAAKYGAAGLGGVLGG AGQFPLGGVAARPGFGLSPIFPGGACLGKACGRKRK); SEQ ID NO: 8 (SEQ ID NO: 8: SAMGGVPGALAAAKAAKYGAAVPGVLGGLGALGGVGIPGGVVGAGPAAAAAAAKAA AKAAQFGLVGAAGLGGLGVGGLGVPGVGGLGGIPPAAAAKAAKYGAAGLGGVLGGA GQFPLGGVAARPGFGLSPIFPGGACLGKACGRKRK); SEQ ID NO: 9 (SEQ ID NO: 9; SAMGALVGLGVPGLGVGAGVPGFGAGADEGVRRSLSPELREGDPSSSQHLPSTPSSPRV PGALAAAKAAKYGAAVPGVLGGLGALGGVGIPGGVVGAGPAAAAAAAKAAAKAAQF GLVGAAGLGGLGVGGLGVPGVGGLGGIPPAAAAKAAKYGAAGLGGVLGGAGQFPLG GVAARPGFGLSPIFPGGACLGKACGRKRK); SEQ ID NO: 10 (SEQ ID NO: 10; GIPPAAAAKAAKYGAAGLGGVLGGAGQFPLGGVAARPGFGLSPIFPGGACLGKACGRK RK); SEQ ID NO: 11 (SEQ ID NO: 11; GAAGLGGVLGGAGQFPLGGVAARPGFGLSPIFPGGACLGKACGRKRK); SEQ ID NO: 12 (SEQ ID NO: 12; GADEGVRRSLSPELREGDPSSSQHLPSTPSSPRV); SEQ ID NO: 13 (SEQ ID NO: 13; GADEGVRRSLSPELREGDPSSSQHLPSTPSSPRF); SEQ ID NO: 14 (SEQ ID NO: 14; AAAGLGAGIPGLGVGVGVPGLGVGAGVPGLGVGAGVPGFGAGADEGVRRSLSPELRE GDPSSSQHLPSTPSSPRVPGALAAAKAAKYGAAVPGVLGGLGALGGVGIPGGVVGAGP AAAAAAAKAAAKAAQFGLVGAAGLGGLGVGGLGVPGVGGLGGIPPAAAAKAAKYGA AGLGGVLGGAGQFPLGGVAARPGFGLSPIFPGGACLGKACGRKRK); SEQ ID NO: 15 (SEQ ID NO: 15; AAAGLGAGIPGLGVGVGVPGLGVGAGVPGLGVGAGVPGFGAVPGALAAAKAAKYGA AVPGVLGGLGALGGVGIPGGVVGAGPAAAAAAAKAAAKAAQFGLVGAAGLGGLGVG GLGVPGVGGLGGIPPAAAAKAAKYGAAGLGGVLGGAGQFPLGGVAARPGFGLSPIFPG GACLGKACGRKRK).

It will be understood that the tropoelastin is provided in the formulations of the embodiments described herein for exploiting the biological activity of tropoelastin in inducing production of mesodermal lineage cells from MSCs. In this context, tropoelastin is an active ingredient of a tropoelastin-containing composition for use in an expansion or differentiation phase.

As discussed above, in some embodiments at least some tropoelastin utilised in cell culture is not attached to a solid phase or hydrogel. This enables at least some, if not all tropoelastin provided in the expansion and/or differentiation phases to appropriately stimulate MSCs for production of MSCs or differentiation of MSCs.

In some embodiments, tropoelastin is in a form in which it is linked to another molecule such as a biopolymer, hyaluronic acid being one example. The linkage may be covalent linkage. In some embodiments, the tropoelastin is cross-linked to hyaluronic acid. In some embodiments, the tropoelastin comprises a sequence set forth in SEQ ID NO: 1.

It is particularly preferred that where tropoelastin is linked to another molecule, the linkage does not impede or limit the biological properties that are inherent in an unlinked form of tropoelastin. Accordingly, where tropoelastin is linked with another molecule, the tropoelastin retains the biological properties of tropoelastin, especially the capacity to be utilised in an expansion or differentiation phase as described herein.

The purpose of linking tropoelastin with another molecule is typically to enable tropoelastin to be localised to a particular region and in particular to minimise the likelihood of the tropoelastin diffusing or otherwise migrating from that region. This is particularly relevant in in vivo embodiments described herein where a depot of tropoelastin is to be provided in an individual to which MSCs are then applied or administered. In some embodiments, the depot of tropoelastin is provided at a local site, wherein the local site is an area of a bone disorder or fracture. In some embodiments, the depot of tropoelastin is provided at a local site, wherein the local site is an area of fat loss or atrophy. In some embodiments, the depot of tropoelastin is provided at a local site, wherein the local site is an area of a cartilage disorder.

It will be understood that in a form where tropoelastin is covalently linked via glutaraldehyde, or by lysyl oxidase (as in elastin), or in an alkaline polymerised form, the tropoelastin has not retained biological activities enabling it to be used in expansion or differentiation phase described herein.

In one embodiment, at least about 50% of the tropoelastin provided for cell culture is linked with a biomolecule and/or biopolymer, such as a saccharide-containing molecule, for example, an oligosaccharide, polysaccharide, or derivatives thereof. In other embodiments, at least about 60%, about 70%, about 80%, about 90% or about 95% tropoelastin or any amount of tropoelastin within a range defined by any two aforementioned values is linked with another molecule.

In the above described embodiments where a complex of tropoelastin and hyaluronic acid is utilised, the hyaluronic acid is utilised at a concentration of generally about 0.1 to 30 mg/ml, more preferably from about 1 mg/ml to about 15 mg/ml.

Preferably in a complex of tropoelastin and hyaluronic acid, the ratio of tropoelastin to hyaluronic acid is about 100:1, more preferably about 50:1, more preferably about 10:1, more preferably about 1:1, more preferably about 1:10, more preferably about 1:100.

In certain embodiments, the number of tropoelastin molecules not linked to another compound in a given composition for use is preferably at least about 5%, about 10%, about 15%, or about 20% of tropoelastin or any amount in range in between any two aforementioned values in a composition.

In certain embodiments, the tropoelastin has a specified degree of purity with respect to the amount of tropoelastin in a composition for cell culture, as compared with amounts of other proteins or molecules in the composition. In one embodiment, the tropoelastin is in a composition that has at least about 75% purity, preferably about 85% purity, more preferably more than about 90% or about 95% purity. Fragments of tropoelastin, i.e., truncated forms of a tropoelastin isoform that arise unintentionally through tropoelastin manufacture may be regarded as an impurity in this context.

It will further be understood that in certain embodiments the tropoelastin may be provided in the form of a composition that consists of or consists essentially of tropoelastin, preferably a full-length isoform of tropoelastin. In alternative embodiments, the tropoelastin will be at least about 65% of the length of the relevant tropoelastin isoform, more than about 80% of the full length, more than about 90% or more than about 95% of the full length.

In certain embodiments, the tropoelastin may be provided for cell culture in the form a 3-dimensional structure. The MSCs may be seeded within the 3D structure or provided in cell culture in conditions enabling the MSCs to migrate to the 3D structure.

A 3D structure may be a hydrogel. Typically, a hydrogel for use according to the some embodiments comprises polymeric hydrophilic molecules forming a scaffold and imbuing the hydrogel with mechanical properties described below, water and tropoelastin for use in an expansion and or an induction phase as described herein.

As described below, examples of polymeric hydrophilic molecules include carboxymethyl cellulose, hydroxypropyl cellulose, hydroxypropyl methylcellulose, hyaluronic acid, xanthan gum, guar gum, β-glucan, alginates, carboxymethyl dextran.

In one embodiment, a hydrogel may provide for a tensile strength of from about 100 kPa to about 2 MPa. Tensile strength is usually defined as the maximum stress that a material can withstand while being stretched or pulled before the material's cross-section starts to significantly stretch. A person skilled in the art will be aware of suitable methods to test the ultimate strength of a material. In some embodiments, the hydrogel can have an ultimate strength ranging from about 10 kPa to about 45 kPa (for example, about 12 kPa to about 40 kPa).

In another embodiment, the hydrogel has a compression strength of from 50 kPa to 700 kPa. Compressive strength is the capacity of a material or structure to withstand axially directed pushing forces. It provides data (or a plot) of force vs deformation for the conditions of the test method. By definition, the compressive strength of a material is that value of uni-axial compressive stress reached when the material fails completely. The compressive strength is usually obtained experimentally by means of a compressive test. The apparatus used for this experiment is the same as that used in a tensile test. However, rather than applying a uni-axial tensile load, a uni-axial compressive load is applied. As can be imagined, the specimen is shortened as well as spread laterally. Compressive strength is often measured on a universal testing machine; these range from very small table-top systems to ones with over 53 MN capacity. Measurements of compressive strength are affected by the specific test method and conditions of measurement.

Compressive strength of the hydrogels can be determined using cyclic loading at a given strain level (for example, about 5%, about 10%, about 15%, about 20%, about 25%, about 30%, about 35%, about 40%, about 45%, about 50%, about 55%, 60%, about 65%, about 70% or about 75% strain level). In some embodiments, the compressive modulus of the hydrogel is about 1 kPa, about 10 kPa, about 20 kPa, about 30 kPa, about 40 kPa, about 50 kPa, about 60 kPa, about 70 kPa, about 80 kPa, about 90 kPa, about 100 kPa, about 110 kPa, about 120 kPa, about 130 kPa, about 140 kPa, about 150 kPa, about 160 kPa, about 170 kPa, 180 kPa, about 190 kPa, about 200 kPa, 210 kPa, about 220 kPa, about 230 kPa, about 240 kPa, about 250 kPa, about 260 kPa, about 270 kPa, 280 kPa, 290 kPa, 300 kPa, about 310 kPa, 320 kPa, 330 kPa, 340 kPa, 350 kPa, about 360 kPa, about 370 kPa, about 380 kPa, about 390 kPa, about 400 kPa, about 410 kPa, about 420 kPa, about 430 kPa, about 440 kPa, about 450 kPa, about 460 kPa, about 470 kPa, about 480 kPa, about 490 kPa, or about 500 kPa or any compressive modulus in between a range defined by any two aforementioned values. The compressive modulus of the hydrogels can range from about 1 kPa to about 500 kPa.

Under compression, the hydrogels can lose energy. Energy loss can range from about 5% to about 50%. In some embodiments, energy loss can be from about 10% to about 40%, from about 20% to about 35% (for example, 23±3.2% or 24.1±7%), or from about 25% to about 30% (for example, 30.5±6.4 or 26.9±2.3).

In one embodiment, the strain at break of the hydrogel is between about 130 kPa and about 420 kPa. The strain at break test is performed by stretching samples until they break and determining the strain at breaking point from the strain/stress curves.

In certain embodiments, the hydrogels or scaffolds may have an elastic modulus of between about 500 Pa to about 50 Pa, about 450 Pa to about 100 Pa, about 400 Pa to about 125 Pa; about 400 Pa to about 150 Pa, or about 385 Pa to about 150 Pa. The elastic modulus will vary depending on the concentration and components used.

In certain embodiments, the hydrogels may have an extrudable length, that is substantially coherent and substantially holds together without support, of at least about 5 cm, about 10 cm, about 12 cm, about 15 cm, about 18 cm, about 20 cm, or about 25 cm when extruded through a 25 G needle or any extrudable length in between a range defined by any two aforementioned values. Certain embodiments may have an extrudable length, that is substantially coherent and substantially holds together without support, of at least about 5 cm, about 10 cm, about 12 cm, about 15 cm, about 18 cm, about 20 cm, or about 25 cm when extruded through a 27 G needle or any extrudable length in between a range defined by any two aforementioned values. Certain embodiments may have an extrudable length, that is substantially coherent and substantially holds together without support, of at least about 5 cm, about 10 cm, about 12 cm, about 15 cm, about 18 cm, about 20 cm, or about 25 cm when extruded through a 30 G needle or 31 G needle or any extrudable length in between a range defined by any two aforementioned values.

Certain embodiments may have an extrudable length of at least about 5 cm, about 10 cm, about 12 cm, about 15 cm, about 18 cm, about 20 cm, about or 25 cm through a fine gauge needle or any extrudable length in between a range defined by any two aforementioned values.

In some embodiments, the hydrogels for use may also be swellable. The term “swellable” refers to hydrogels that are substantially insoluble in a swelling agent and are capable of absorbing a substantial amount of the swelling agent, thereby increasing in volume when contacted with the swelling agent. As used herein, the term “swelling agent” has its plain and ordinary meaning in view of the paper and without limitation may refer to those compounds or substances which produce at least a degree of swelling. Typically, a swelling agent is an aqueous solution or organic solvent, however the swelling agent can also be a gas. In some embodiments, a swelling agent is water or a physiological solution, for example phosphate buffer saline, or growth media.

In some embodiments, the hydrogel comprises a swelling agent. In some embodiments, the hydrogel can contain over 50% (w/v), over 60% (w/v), over 70% (w/v), over 80% v, over 90% (w/v), over 91% (w/v), over 92% (w/v), over 93% (w/v), over 94% (w/v), over 95% (w/v), over 96% (w/v), over 97% v, over 98% (w/v), over 99% (w/v), or more of the swelling agent.

The term “swelling ratio” is used herein to mean weight of swelling agent in swollen hydrogel per the dried weight of the hydrogel before swelling. For example, the swelling ratio can range from about 1 to about 10 grams of swelling agent per gram of the tropoelastin in the hydrogel. In some embodiments, the swelling ratio can be from about 1 to about 5 grams of swelling agent per gram of the tropoelastin in the hydrogel. In some embodiments, the swelling ratio can be about 1.25, about 1.5, about 1.75, about 2, about 2.25, about 2.5, about 2.75, about 3, about 3.25, about 3.5, about 3.75, about 4, about 4.25, about 4.5, about 4.75 or about 5 grams of swelling agent per gram of tropoelastin in the hydrogel. In some embodiments, the swelling ratio can be 1.2±0.2, 2.3±0.3, or 4.1±0.3 grams of swelling agent per gram of tropoelastin in the hydrogel.

In certain preferred embodiments, the hydrogels comprise Hyaluronic acid (HA) for use as a scaffold. In these circumstances, the HA functions to provide certain mechanical properties to the hydrogel, allowing the tropoelastin to remain substantially free (un-crosslinked), such that the tropoelastin has the ability to function as a biological factor, stimulating and inducing bone formation at the site where the hydrogel is provided.

In certain embodiments, where the hydrogel includes tropoelastin and hyaluronic acid, the mass ratio of tropoelastin to hyaluronic acid is about 0.1:1 to about 500:1, preferably, about 0.2:1 to about 100:1.

In yet further embodiments, the hydrogel may comprise HA in a concentration of between about 0.1% to about 15%. In certain embodiments, the hydrogel may comprise the HA in a concentration of between about 0.1% to about 10%.

The hydrogel may comprise derivatised HA or underivatized HA, to control the extent to which the HA crosslinks with itself and/or the monomeric protein.

In certain embodiments, the HA may comprise, at least one linkable moiety, such as at least one cross-linkable moiety, for example, a carboxyl group, a hydroxyl group, an amine, a thiol, an alcohol, an alkene, an alkyne, a cyano group, or an azide, and/or modifications, derivatives, or combinations thereof.

In certain embodiments, the HA may comprise, a spacer group, such that the spacer group is capable of linking to the same and/or a second molecule, for example, a second biomolecule or biopolymer.

The HA used in the hydrogel may be in the range of about 50 to about 4000 disaccharide units or residues, for example about 2000 to 2500 disaccharide units or residues. In other embodiments, hyaluronic acid may be used in the range of 200 to 10,000 disaccharide units or residues.

In certain embodiments, the HA may be low or high molecular weight, and the choice of which will vary depending on the skilled person's intentions for modifying the viscosity of the hydrogel. For example, use of lower molecular weight hyaluronic acid allows the hyaluronic acid to be modified, precipitated and washed and the hyaluronic acid remains a reasonably low viscous solution that may be readily used as the cross-linking agent. Using higher molecular weight polysaccharides may provide additional handling issues (e.g., viscous solution, problems with mixing, aeration etc) but, in certain embodiments, a wide range of molecular weights may be used to achieve the desired results.

In certain embodiments, the HA may be activated and/or modified with an activating agent, such as EDC or allylglycidyl ether, and/or modifying agent, such as NHS, HOBt or Bromine.

The term “hyaluronic acid” or “HA” may include hyaluronic acid and any of its hyaluronate salts, including, for example, sodium hyaluronate (the sodium salt), potassium hyaluronate, magnesium hyaluronate, and calcium hyaluronate. Hyaluronic acid from a variety of sources may be used herein. For example, hyaluronic acid may be extracted from animal tissues, harvested as a product of bacterial fermentation, or produced in commercial quantities by bioprocess technology.

Suitable polysaccharides which may also be included in the hydrogels include carboxy cellulose, carboxymethyl cellulose, hydroxymethyl cellulose, hydroxypropyl cellulose (HPC), hydroxypropyl methylcellulose (HPMC), hydroxy-propylcellulosecarboxymethyl amylose (“CMA”), xanthan gum, guar gum, β-glucan, alginates, carboxymethyl dextran, a glycosaminoglycan derivative, chondroitin-6-sulfate, dermatin sulfate, polylactic acid (PLA), or biomaterials such as polyglycolic acid (PGA), poly(lactic-co-glycolic) acid (PLGA), tricalcium phosphate (TCP), 1-hydroxyapatite (PAH), and their pharmaceutically acceptable salts.

Alternatively, the polysaccharide may be a pectin or a derivative thereof, including linear and branched polysaccharides.

When the scaffold agents used in the tropoelastin hydrogels is carboxymethylcellulose or xanthan gum, the agent may be provided in an amount of from about 0.01 to about 10 percent (w/v), preferably in an amount of from about 0.5 to about 3.5 percent (w/v).

The scaffold may be a cross-linked or uncross-linked polysaccharide typically having a substitution or additional side chain.

Additional scaffold may include scaffolds derived from polymethacrylates, polyethylene glycols and (block) copolymers with polyethylene glycol subunits (for example Poloxamer 188 and Poloxamer 407). Alternative agents included in the hydrogels include surfactants such as sodium lauryl sulfate and polysorbates, or pantothenol, polyethylene glycols, xanthan gum, guar gum, polysorbate 80, N-acetylglucosamine and their pharmaceutically acceptable salts.

### Additional Embodiments

In some embodiments, a method of forming cells of mesodermal lineage from MSCs is provided. The method can comprise the steps of (i) providing a cell culture vessel having a cell culture surface, the cell culture surface having tropoelastin arranged thereon, said arrangement enabling tropoelastin to at least partially dissolve in a cell culture medium for culture of an MSC; and (ii) culturing MSCs in the culture vessel and thereby forming cells of mesodermal lineage from MSCs.

In some embodiments, osteogenesis, adipogenesis and chondrogenesis is promoted due to the presence of tropoelastin in the expansion state. This effect may be separate from the mitogenic effect of tropoelastin. In some embodiments, osteogenesis, adipogenesis and chondrogenesis is promoted when cells are exposed to tropoelastin at the expansion stage. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In the methods of the embodiments described herein, the tropoelastin may replace a proliferation factor in full serum media. In some embodiments, the tropoelastin not only improves MSC propagation in normal or growth factor-supplemented media but can also replace either IGF-1 or bFGF while maintaining the same amplified level of cell expansion. In some embodiments, the tropoelastin may replace proliferation factor in reduced serum media. In some embodiments, the tropoelastin enables substantial serum reduction in media. Tropoelastin may be used to reduce the reliance on serum during MSC expansion, which is clinically beneficial and may avoid infection risks from an animal-derived product such as, for example, an adverse immune response. As such, tropoelastin may be used for culturing clinically relevant cells. In some embodiments, tropoelastin at a concentration of at least 1 μg/mL also significantly enhances MSC expansion. In some embodiments, the tropoelastin allows for greater serum reduction compared to growth factors. In some embodiments, tropoelastin in solution promotes MSC proliferation similarly to surface-bound tropoelastin. In some embodiments, tropoelastin in solution can replace IGF-1 and bFGF in full serum media. In some embodiments, at higher concentrations of tropoelastin equivalent to the substrate coating concentration, tropoelastin in solution functionally supersedes the surface-bound protein and parallels the synergistic effect of IGF-1 and bFGF in full serum media. In some embodiments, tropoelastin improves MSC propagation in normal or growth factor-supplemented media. In some embodiments, tropoelastin improves cell expansion. The tropoelastin in the embodiments herein, allows MSCs to retain cell phenotype during tropoelastin-mediated expansion. In some embodiments herein, the tropoelastin modulates MSC attachment and spreading via αv integrins. In some embodiments herein, the tropoelastin modulates MSC expansion via αv integrins. In some embodiments of the methods described herein, the substrate-bound and soluble tropoelastin attract MSCs. In some embodiments, wherein the tropoelastin is added to a solution, the tropoelastin is prevented from adhesion to a solid phase, wherein the solid phase is a vehicle for holding the cells, such as a cell culture vessel. A tissue culture substrate, such as a cell culture vessel, may be coated with a protein, for example, in order to prevent adhesion of a second protein, such as tropoelastin in solution, from adhering to the tissue culture substrate. The protein used for coating may be serum proteins, for example. Excess serum proteins may be washed away before performing the cell culturing techniques. In some embodiments, the proliferation factors and/or differentiation factors promote MSC differentiation during osteogenesis. In some embodiments, the promotion of osteogenesis, adipogenesis and chondrogenesis is enhanced when the MSCs are exposed to tropoelastin in the expansion stage. In some embodiments, the tropoelastin does not have a mitogenic effect on the MSCs. In some embodiments, a method for treating an individual having a bone disorder or fracture is provided, wherein the method comprises providing a composition according to any one of the compositions of the embodiments as described herein to the individual, thereby treating the individual for a bone disorder or fracture. In some embodiments, the composition is formed by any one of the methods described in the embodiments herein. In some embodiments, the method of forming the cells comprises contacting MSCs with at least one differentiation factor for inducing formation of cells of mesodermal lineage from MSC and tropoelastin wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises (i) culturing MSCs in a medium containing tropoelastin to form a tropoelastin-cultured MSC population; and (ii) culturing said tropoelastin-cultured MSC population in a medium, wherein the medium includes at least one differentiation factor for inducing differentiation of an MSC. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the step (i) is performed in the absence of in the absence of IGF-1 and bFGF. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the cell of mesodermal lineage is an osteocyte, chondrocyte or adipocyte. In some embodiments, the MSCs are human MSCs. In some embodiments, the composition is a substantially pure form of osteocytes. In some embodiments, the composition includes tropoelastin and/or hyaluronic acid. In some embodiments, the tropoelastin is cross-linked to the hyaluronic acid. In some embodiments, the individual is provided the composition, wherein the amount of total MSC provided to the individual is at least one to two million cells per kilogram of body weight of the individual. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method for forming cells of mesodermal lineage from mesenchymal stem cells (MSC) is provided, wherein the method comprises (i) contacting MSCs with tropoelastin during an expansion phase to induce proliferation of MSCs, thereby forming an expanded population of MSCs; and (ii) contacting the expanded population of MSCs with tropoelastin during a differentiation phase. In some embodiments, the method further comprises contacting the MSCs during the expansion phase with at least one factor for inducing expansion or proliferation of MSCs. In some embodiments, the at least one factor for inducing expansion or proliferation of MSCs comprises TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the at least one factor for inducing expansion or proliferation of MSCs comprises IGF-1 and/or bFGF. In some embodiments, the method further comprises contacting the MSCs during the differentiation phase with differentiation factors. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, exposure to tropoelastin during MSC expansion and induction may modulate a cell's functional differentiation into bone (osteogenesis), fat (adipogenesis) and cartilage (chondrogenesis). In some embodiments, the presence of tropoelastin during MSC expansion improves osteogenesis in comparison to osteogenesis in cells that are not exposed to tropoelastin. In some embodiments, tropoelastin addition during expansion and differentiation increases osteogenesis as compared to cells that have not been exposed to tropoelastin during expansion and differentiation stages. In some embodiments, tropoelastin addition during MSC expansion or differentiation increases adipogenesis as compared to cells that have not been exposed to tropoelastin during MSC expansion and differentiation. In some embodiments, benefits are seen with an uninterrupted tropoelastin presence. In some embodiments, the presence of tropoelastin during MSC expansion improves chondrogenesis as compared to MSC cells that are not exposed to tropoelastin during MSC expansion. In some embodiments, the MSCs are exposed to tropoelastin from days 1-7 of a seven-day expansion period. In some embodiments, the MSCs are exposed to tropoelastin from days 2-5 of a seven-day expansion period. In some embodiments, the MSCs are exposed to tropoelastin from days 4-7 of a seven-day expansion period. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method of forming osteocytes from MSCs is provided, wherein the method comprises (i) contacting MSCs during an expansion phase with tropoelastin to induce proliferation of MSCs, thereby forming an expanded population of MSCs; and (ii) contacting the expanded population of MSCs with tropoelastin and at least one factor for inducting formation of osteocytes from MSCs during a differentiation phase. In some embodiments, the method further comprises contacting the MSCs during the expansion phase with at least one factor for inducing expansion or proliferation of MSCs. In some embodiments, the at least one factor for inducing expansion or proliferation of MSCs comprises TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the at least one factor for inducing expansion or proliferation of MSCs comprises IGF-1 and/or bFGF. In some embodiments, the at least one factor for inducing formation of osteocytes from MSCs comprises dexamethasone, ascorbate and/or beta-glycerophosphate. In some embodiments, the expansion phase is performed completed independently of the differentiation phase. The presence of tropoelastin leads to an increased efficacy of osteogenic differentiation as compared to a method performed in the absence of tropoelastin. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method of forming adipocytes from MSCs is provided, the method comprising (i) contacting MSCs during an expansion phase with tropoelastin to induce proliferation of MSCs, thereby forming an expanded population of MSCs; and (ii) contacting the expanded population of MSCs with tropoelastin and at least one factor for inducing formation of adipocytes from MSCs during a differentiation phase. In some embodiments, the method further comprises contacting the MSCs during the expansion phase with at least one factor for inducing expansion or proliferation of MSCs. In some embodiments, the at least one factor for inducing expansion or proliferation of MSCs comprises TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the at least one factor for inducing expansion or proliferation of MSCs comprises IGF-1 and/or bFGF. In some embodiments, the at least one factor for inducing formation of adipocytes from MSCs comprises h-insulin, dexamethasone, indomethacin and/or 3-isobutyl-1-methylxanthine. In some embodiments, the expansion phase is completed independently of the differentiation phase. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method of forming chondrocytes from MSCs is provided, the method comprising (i) contacting MSCs during an expansion phase with tropoelastin to induce proliferation of MSCs, thereby forming an expanded population of MSCs; and (ii) contacting the expanded population of MSCs with tropoelastin and at least one factor for inducing formation of chondrocytes from MSCs during a differentiation phase. In some embodiments, the method further comprises contacting the MSCs during the expansion phase with at least one factor for inducing expansion or proliferation of MSCs. In some embodiments, the at least one factor for inducing expansion or proliferation of MSCs comprises TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, the at least one factor for inducing expansion or proliferation of MSCs comprises IGF-1 and/or bFGF. In some embodiments, the at least one factor for inducing formation of chondrocytes from MSCs comprises dexamethasone, ascorbate, insulin-transferrin-selenium, sodium pyruvate and/or proline. In some embodiments, the expansion phase is completed independently of the differentiation phase. In some embodiments, the tropoelastin is not provided with silk protein. In some embodiments, the tropoelastin is provided in a form of a complex with hyaluronic acid, wherein the hyaluronic acid is partially or completely soluble and wherein the tropoelastin is in a monomeric form linked together by hyaluronic acid. In some embodiments, the tropoelastin is cross-linked to the hyaluronic acid. In some embodiments, the MSCs are human MSCs. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method of forming osteocytes from MSCs is provided, wherein the method comprises (i) contacting MSCs during an expansion phase with tropoelastin to induce proliferation of MSCs, thereby forming an expanded population of MSCs; and (ii) contacting the expanded population of MSCs with tropoelastin and at least one factor for inducting formation of osteocytes from MSCs during a differentiation phase. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, step (i) is performed in the absence of IGF-1 and/or bFGF. In some embodiments, the at least one factor for inducing formation of osteocytes from MSCs comprises dexamethasone, ascorbate and/or beta-glycerophosphate. In some embodiments, the expansion phase is performed completed independently of the differentiation phase. The presence of tropoelastin leads to an increased efficacy of osteogenic differentiation as compared to a method performed in the absence of tropoelastin. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method of forming adipocytes from MSCs is provided, the method comprising (i) contacting MSCs during an expansion phase with tropoelastin to induce proliferation of MSCs, thereby forming an expanded population of MSCs; and (ii) contacting the expanded population of MSCs with tropoelastin and at least one factor for inducing formation of adipocytes from MSCs during a differentiation phase. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, step (i) is performed in the absence of IGF-1 and/or bFGF. In some embodiments, the at least one factor for inducing formation of adipocytes from MSCs comprises h-insulin, dexamethasone, indomethacin and/or 3-isobutyl-1-methylxanthine. In some embodiments, the expansion phase is completed independently of the differentiation phase. In some embodiments, cells that are exposed to tropoelastin exhibit an increase in intracellular lipid formation in the presence of tropoelastin as compared to the culture lacking tropoelastin. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a method of forming chondrocytes from MSCs is provided, the method comprising (i) contacting MSCs during an expansion phase with tropoelastin to induce proliferation of MSCs, thereby forming an expanded population of MSCs; and (ii) contacting the expanded population of MSCs with tropoelastin and at least one factor for inducing formation of chondrocytes from MSCs during a differentiation phase. In some embodiments, step (i) is performed in the absence of TGFβ1, TGFβ2, TGFβ3, BMP-2, BMP-3, BMP-4, BMP-5, BMP-6, BMP-7, bFGF, FGF-4, EGF, IGF-1, PDGF-A, PDGF-B, PDGF-C, PDGF-D, HGF, VEGF, VEGF-A and/or Wnt3a. In some embodiments, step (i) is performed in the absence of IGF-1 and/or bFGF. In some embodiments, the at least one factor for inducing formation of chondrocytes from MSCs comprises dexamethasone, ascorbate, insulin-transferrin-selenium, sodium pyruvate and/or proline. In some embodiments, the expansion phase is completed independently of the differentiation phase. In some embodiments, the tropoelastin is not provided with silk protein. In some embodiments, the tropoelastin is provided in a form of a complex with hyaluronic acid, wherein the hyaluronic acid is partially or completely soluble and wherein the tropoelastin is in a monomeric form linked together by hyaluronic acid. In some embodiments, the tropoelastin is cross-linked to the hyaluronic acid. In some embodiments, the MSCs are human MSCs. In some embodiments, cells exposed to tropoelastin in step (i) exhibit increased glycosaminoglycan levels as compare to cells that are expanded without tropoelastin. In some embodiments, the presence of tropoelastin during the differentiation phase increases the efficacy of differentiation. In some embodiments, tropoelastin added during expansion and/or differentiation improves the differentiation potential. In some embodiments, a tropoelastin concentration of 5 ug/ml, 10 ug/ml, 15 ug/ml, 20 ug/ml or 25 ug/ml or any concentration in between a range defined by any two aforementioned values is added during the expansion and/or differentiation phase.

In some embodiments, a composition comprising cells manufactured by anyone of the embodiments described herein is provided. In some embodiments, the composition comprises a substantially pure form of osteocytes. In some embodiments, the composition comprises a substantially pure form of adipocytes. In some embodiments, the composition comprises a substantially pure form of chondrocytes. In some embodiments, the composition further comprises tropoelastin and/or hyaluronic acid. In some embodiments, the tropoelastin is cross-linked to the hyaluronic acid.

In some embodiments, a method of treating an individual having a bone disorder or fracture is provided, wherein the method comprises providing the composition of anyone of the embodiments described herein to the individual. In some embodiments, the composition further comprises tropoelastin. In some embodiments, the composition further comprises at least one factor for differentiation of an MSC to form an osteocyte or precursor of an osteocyte. In some embodiments, the composition is administered to the individual at a local site, wherein the local site is an area of the bone disorder or fracture.

In some embodiments, a method of treating an individual having a region of fat loss or atrophy arising from a disease or trauma, or an individual requiring surgical enhancement arising from surgery or disease is provided, wherein the method comprises providing a composition of anyone of the embodiments described herein to the individual. In some embodiments, the composition further comprises tropoelastin. In some embodiments, the composition further comprises at least one factor for differentiation of an MSC to form an adipocyte or precursor of an adipocyte. In some embodiments, the composition is administered to the individual at a local site, wherein the local site is an area of the fat loss or atrophy.

In some embodiments, a method of treating an individual having a cartilage disorder is provided, wherein the method comprises providing the composition of anyone of the embodiments described herein to the individual. In some embodiments, the composition further comprises tropoelastin. In some embodiments, the composition further comprises at least one factor for differentiation of an MSC to form a chondrocyte or precursor of a chondrocyte. In some embodiments, the composition is administered to the individual at a local site, wherein the local site is an area of the cartilage disorder.

In some embodiments, a method of inducing proliferation of MSCs is provided, the method comprises contacting MSCs with tropoelastin to induce proliferation of MSCs, wherein the number of MSCs formed in the presence of tropoelastin is greater than the number of MSCs formed in the absence of tropoelastin, thereby inducing proliferation of MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises (i) culturing MSCs in a medium containing tropoelastin to form a tropoelastin-cultured MSC population; and (ii) culturing said tropoelastin-cultured MSC population in a medium including a factor for inducing proliferation of an MSC. In some embodiments, tropoelastin is present during an expansion phase and in an absence of a factor for inducing expansion or proliferation of an MSC. In some embodiments, the method is performed in an absence of IGF1 and/or bFGF. In some embodiments, an expansion phase is performed in absence of tropoelastin and in absence of a protein source. In some embodiments, the protein source is from serum.

In some embodiments, a method for forming a cell of mesodermal lineage from an MSC is provided, wherein the method comprises (i) administering tropoelastin to an individual requiring formation of cells of mesodermal lineage from an MSC thereby forming a depot of tropoelastin in the individual; and (ii) administering MSCs to the individual so that the MSCs contact the depot of tropoelastin; thereby forming a cell of mesodermal lineage from an MSC. The MSCs are administered locally into a region in need of cells. In some embodiments, the individual is suffering from fat loss or atrophy arising from a disease or trauma. In some embodiments, the individual is suffering from a bone disorder or a fracture. In some embodiments, the individual is suffering from a cartilage disorder.

In some embodiments, a method for forming cells of mesodermal lineage from mesenchymal stem cells (MSC) is provided. The method comprises contacting MSCs with: (i) at least one differentiation factor for inducing formation of cells of mesodermal lineage from MSC and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises: (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the tropoelastin is monomeric. In some embodiments, the tropoelastin is cross-linked to the hyaluronic acid. In some embodiments, the cell of mesodermal lineage is an osteocyte, chondrocyte or adipocyte. In some embodiments, the MSCs are human MSCs.

In some embodiments, a composition of cells formed from method according to any one of the embodiments herein is provided. The method of forming cells comprises contacting MSCs with: (i) at least one differentiation factor for inducing formation of cells of mesodermal lineage from MSC and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises: (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the tropoelastin is monomeric. In some embodiments, the tropoelastin is cross-linked to the hyaluronic acid. In some embodiments, the cell of mesodermal lineage is an osteocyte, chondrocyte or adipocyte. In some embodiments, the MSCs are human MSCs. In some embodiments, the composition is a substantially pure form of osteocytes. In some embodiments, the composition includes tropoelastin and/or hyaluronic acid.

In some embodiments, a method for treating an individual having a bone disorder or fracture is provided. The method comprises providing a composition according to any one of the embodiments herein, to the individual, thereby treating the individual for a bone disorder or fracture. The composition of cells is formed from a method according to any one of the embodiments herein. The method of forming cells comprises contacting MSCs with: (i) at least one differentiation factor for inducing formation of cells of mesodermal lineage from MSC and (ii) tropoelastin, wherein the number of cells of mesodermal lineage formed from MSC in the presence of tropoelastin is greater than the number of cells of mesodermal lineage formed in the absence of tropoelastin, thereby forming cells of mesodermal lineage from MSCs. In some embodiments, the tropoelastin is arranged on a cell culture surface of a cell culture vessel to enable the MSCs to contact the tropoelastin when the MSCs are contacted with the cell culture surface. In some embodiments, the tropoelastin is partially or fully solubilized in a cell culture medium for culture of an MSC. In some embodiments, the method further comprises: (i) contacting MSCs with tropoelastin in the absence of factors that induce differentiation to induce proliferation of MSCs, thereby forming a population of MSCs; and (ii) contacting the population of MSCs with at least one differentiation factor for inducing formation of cells of mesodermal lineage from MSC and tropoelastin. In some embodiments, the method further comprises: (i) culturing MSCs in a first medium containing tropoelastin to form a tropoelastin-cultured MSC population; and (ii) culturing said tropoelastin-cultured MSC population in a second medium, wherein the second medium includes at least one differentiation factor for inducing differentiation of an MSC. In some embodiments, the tropoelastin is not provided with silk protein. In some embodiments, the tropoelastin is provided in the form of a complex with hyaluronic acid that is partially or completely soluble, wherein the tropoelastin monomers are linked together by hyaluronic acid. In some embodiments, the tropoelastin is monomeric. In some embodiments, the tropoelastin is cross-linked to the hyaluronic acid. In some embodiments, the cell of mesodermal lineage is an osteocyte. In some embodiments, the MSCs are human MSCs. In some embodiments, the composition is a substantially pure form of osteocytes. In some embodiments, the composition includes tropoelastin and/or hyaluronic acid. In some embodiments, the individual is provided the composition, wherein the amount of total MSC provided to the individual in the composition is at least one to two million cells per kilogram of body weight of the individual. In some embodiments, the individual is provided the composition, wherein the amount of total MSC provided to the individual in the composition is at least one to two million cells, and wherein the composition is administered to a local site. Thus, cells that are pretreated with tropoelastin are used in the treatment of a bone disorder.

In some embodiments, a cell culture medium comprising tropoelastin is provided, wherein the medium does not contain insulin-like growth factor-1 (IGF-1) and/or basic fibroblast growth factor growth factor (bFGF). In some embodiments, the medium comprises about 2.5 μg/mL to about 20 μg/mL tropoelastin. In some embodiments, the medium comprises about 2% to about 10% serum. In some embodiments, the medium comprises about 2% to about 6% serum. In some embodiments, the serum is fetal bovine serum (FBS). In some embodiments, the medium is serum-free. In some embodiments, the medium comprises minimal essential medium (MEM). In some embodiments, the medium comprises L-glutamine. In some embodiments, the medium comprises about 2.5 μg/mL to about 20 μg/mL tropoelastin, about 2% to about 10% FBS, minimal essential medium (MEM), and L-glutamine.

In some embodiments, a cell culture medium is provided, wherein the cell culture medium comprises tropoelastin, wherein the cell culture medium does not contain an additional factor for inducing expansion or proliferation of MSCs is provided. In some embodiments, the cell culture medium is absent of insulin-like growth factor-1 (IGF-1) and/or basic fibroblast growth factor growth factor (bFGF). In some embodiments, the cell culture medium is absent of TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the cell culture medium comprises about 2.5 μg/mL to about 20 μg/mL tropoelastin. In some embodiments, the cell culture medium comprises about 2% to about 10% serum. In some embodiments, the cell culture medium comprises about 2% to about 6% serum. In some embodiments, the serum is fetal bovine serum (FBS). In some embodiments, the cell culture medium is serum-free. In some embodiments, the cell culture medium comprises minimal essential medium (MEM). In some embodiments, the medium comprises L-glutamine. In some embodiments, the cell culture medium comprises about 2.5 μg/mL to about 20 μg/mL tropoelastin, about 2% to about 10% FBS, minimal essential medium (MEM), and L-glutamine. In some embodiments, the tropoelastin is provided in the form of a complex with hyaluronic acid. In some embodiments, the tropoelastin is cross-linked to the hyaluronic acid.

In some embodiments, a cell culture is provided, wherein the cell culture comprises mesenchymal stem cells; and a medium comprising tropoelastin, wherein the medium does not contain an additional factor for inducing expansion or proliferation of MSCs. In some embodiments, the medium does not contain insulin-like growth factor-1 (IGF-1) and/or basic fibroblast growth factor growth factor (bFGF). In some embodiments, the factor for inducing expansion or proliferation of MSCs comprises TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the mesenchymal stem cells are human mesenchymal stem cells. In some embodiments, the medium comprises about 2.5 μg/mL to about 20 μg/mL tropoelastin. In some embodiments, the tropoelastin is provided in the form of a complex with hyaluronic acid. In some embodiments, the medium comprises about 2% to about 10% serum or about 2% to about 6% serum. In some embodiments, the medium is serum-free. In some embodiments, the medium comprises about 2.5 μg/mL to about 20 μg/mL tropoelastin, about 2% to about 10% FBS, minimal essential medium (MEM), and L-glutamine.

In some embodiments, a cell culture medium is provided, wherein the cell culture medium comprises at least one differentiation factor; and tropoelastin. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

In some embodiments, a cell culture comprising: mesenchymal stem cells; and a medium comprising tropoelastin, wherein the medium does not contain an additional factor for inducing expansion or proliferation of MSCs, is provided. In some embodiments, the factor for inducing expansion or proliferation of MSCs comprises wherein the factor for inducing expansion or proliferation of MSCs comprises TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the mesenchymal stem cells are human mesenchymal stem cells. In some embodiments, the medium comprises about 2.5 μg/mL to about 20 μg/mL tropoelastin. In some embodiments, the tropoelastin is provided in the form of a complex with hyaluronic acid. In some embodiments, the tropoelastin is cross-linked to the hyaluronic acid. In some embodiments, the medium comprises 2% to about 10% serum or about 2% to about 6% serum. In some embodiments, the medium is serum-free. In some embodiments, the medium comprises about 2.5 μg/mL to about 20 μg/mL tropoelastin, about 2% to about 10% FBS, minimal essential medium (MEM), and L-glutamine.

In some embodiments, a cell culture is provided, wherein the cell culture comprises mesenchymal stem cells; and a medium comprising tropoelastin and at least one differentiation factor. In some embodiments, the at least one differentiation factor comprises dexamethasone, ascorbate and/or beta-glycerophosphate. In some embodiments, the at least one differentiation factor comprises h-insulin, dexamethasone, indomethacin and/or 3-isobutyl-1-methyl-xanthine. In some embodiments, the at least one differentiation factor comprises dexamethasone, ascorbate, insulin-transferrin-selenium, sodium pyruvate and/or proline.

In some embodiments, a method for culturing a mesenchymal stem cell is provided, the method comprising: a) culturing a mesenchymal stem cell in a cell culture medium, wherein the medium does not contain an additional factor for inducing expansion or proliferation of MSCs; and b) expanding the mesenchymal stem cell in the presence of tropoelastin. In some embodiments, the mesenchymal stem cell is exposed to tropoelastin from days 1-7, days 2-5, or days 4-7 of a seven-day expansion period. In some embodiments, the factor for inducing expansion or proliferation of MSCs comprises TGFβ1, TGFβ2, TGFβ3, BMP-2, BMP-3, BMP-4, BMP-5, BMP-6, BMP-7, basic fibroblast growth factor (bFGF), FGF-4, EGF, insulin-like growth factor 1 (IGF-1), PDGF-A, PDGF-B, PDGF-C, PDGF-D, HGF, VEGF, VEGF-A or Wnt3a. In some embodiments, the additional factor for inducing expansion or proliferation comprises IGF-1 and bFGF. In some embodiments, the mesenchymal stem cells are human mesenchymal stem cells. In some embodiments, the medium comprises about 2.5 μg/mL to about 20 μg/mL tropoelastin. In some embodiments, the tropoelastin is provided in the form of a complex with hyaluronic acid. In some embodiments, the tropoelastin is cross-linked to the hyaluronic acid. In some embodiments, the medium comprises about 2% to about 10% serum. In some embodiments, the medium is serum-free. In some embodiments, the method further comprises differentiating the mesenchymal stem cells in a medium comprising at least one differentiation factor. In some embodiments, the presence of tropoelastin increases the efficacy of differentiation.

### EXAMPLES

### Example 1

### Surface-Bound Tropoelastin can Replace Either IGF-1 or bFGF in Full Serum Media

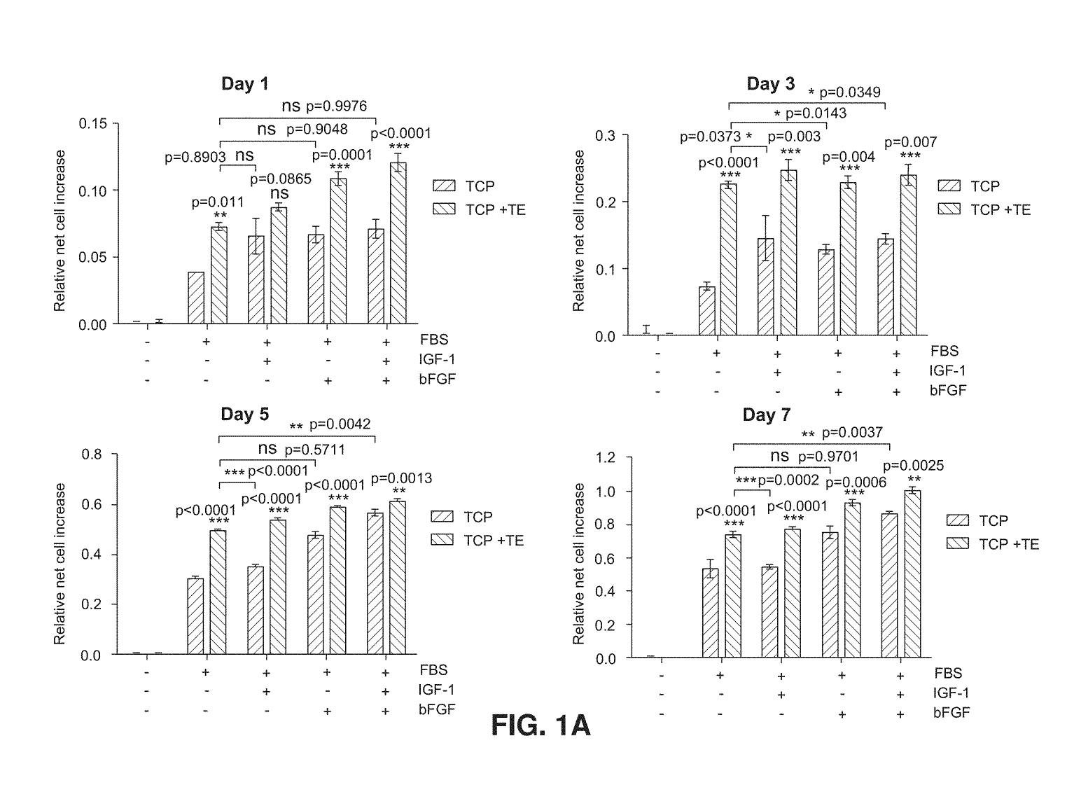

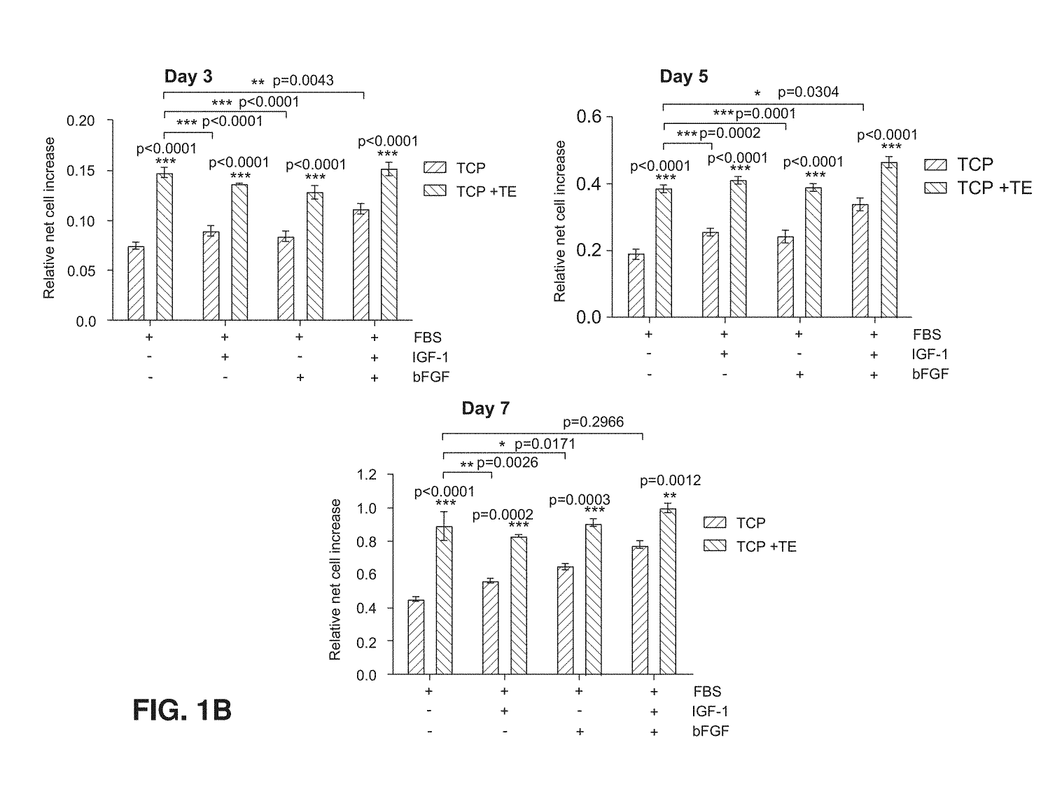

To determine the effect of substrate-bound tropoelastin on MSC proliferation, MSCs were cultured on bare or tropoelastin-coated tissue culture plastic (TCP) in various media formulations with and without 10% fetal bovine serum (FBS), and optionally supplemented with IGF-1 and/or bFGF (FIG. 1A). Cells proliferated over 7 days in all conditions except in serum-free basal media. In normal 10% (v/v) serum-containing media, cell numbers on tropoelastin-coated TCP increased 39±3% more than those on bare TCP. A significant tropoelastin-mediated proliferative increase of 41±1%, 16±2%, and 16±3% was also observed even in IGF-1, bFGF, or IGF-1 and bFGF supplemented media, respectively. The highest cell numbers were observed in the presence of both surface-bound tropoelastin and soluble growth factors.

Comparing the pro-proliferative activity of tropoelastin and growth factors, MSCs cultured on a tropoelastin substrate in normal media with no additional factors displayed 14±2% decreased expansion compared to cells on TCP in media containing both IGF-1 and bFGF. However, cells grown on tropoelastin in normal media proliferated 36±3% more than cells on TCP in media with IGF-1, and similarly to cells in media with bFGF. These findings indicate that substrate-bound tropoelastin not only improves MSC propagation in normal or growth factor-supplemented media but can also replace either IGF-1 or bFGF while maintaining the same amplified level of cell expansion.

### Example 2

### Substrate-Bound Tropoelastin can Replace Both IGF-1 and bFGF in Reduced Serum Media

The addition of growth factors in culture media typically allows for a decrease in serum concentration without retarding MSC proliferation. Therefore, experiments were performed to determine the pro-proliferative benefits of substrate-bound tropoelastin in a reduced serum environment normally compensated for by growth factors (FIG. 1B). In media containing 7% FBS, MSCs grown on TCP also exhibited proliferation over 7 days, although to a lesser extent than that previously observed in normal full serum media. Substrate-bound tropoelastin dramatically promoted MSC proliferation in all reduced serum conditions, not only in unsupplemented media (97±19% increase), but also in media already containing IGF-1, bFGF, or both growth factors (49±1%, 40±3%, or 29±3% increase, respectively).

More remarkably, in these reduced serum conditions, MSCs cultured on tropoelastin in unsupplemented media exhibited significantly greater expansion over 7 days relative to cells on TCP in media with either IGF-1 or bFGF (59±15% and 37±13% increase, respectively), and were equivalent in abundance to cells in media with both growth factors. These results point to the ability of surface-coated tropoelastin to replace both IGF-1 and bFGF in promoting MSC proliferation in a reduced serum environment.

### Example 3

### Tropoelastin Enables Substantial Serum Reduction in Media

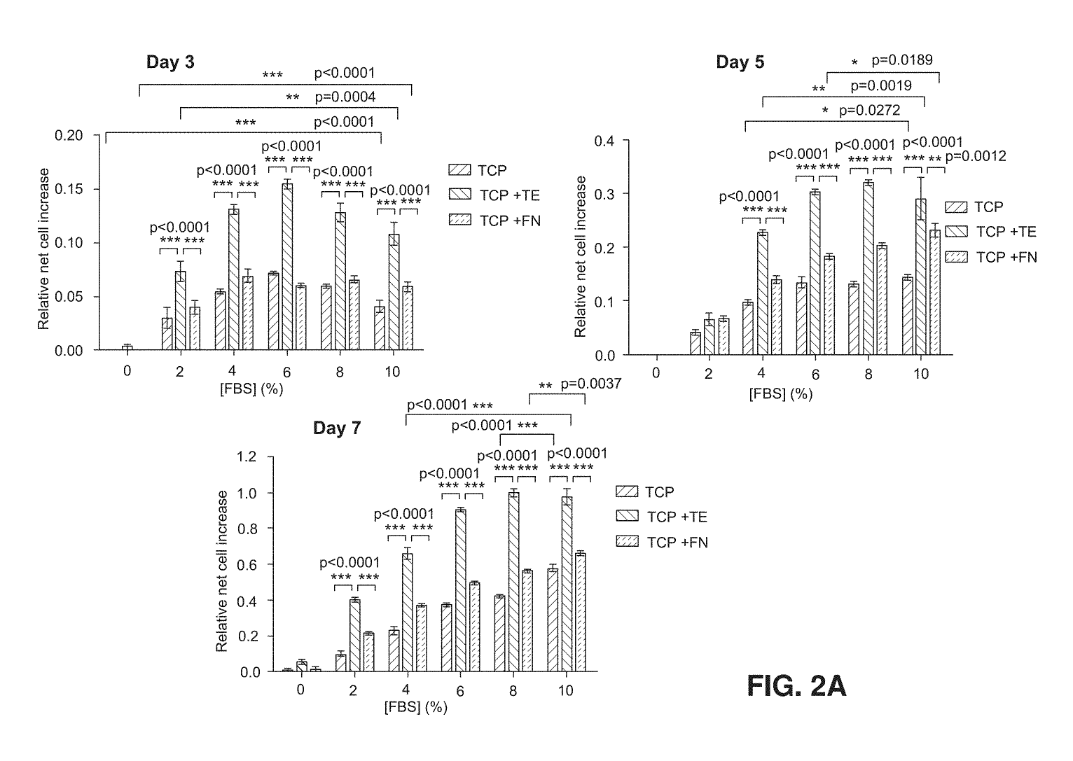

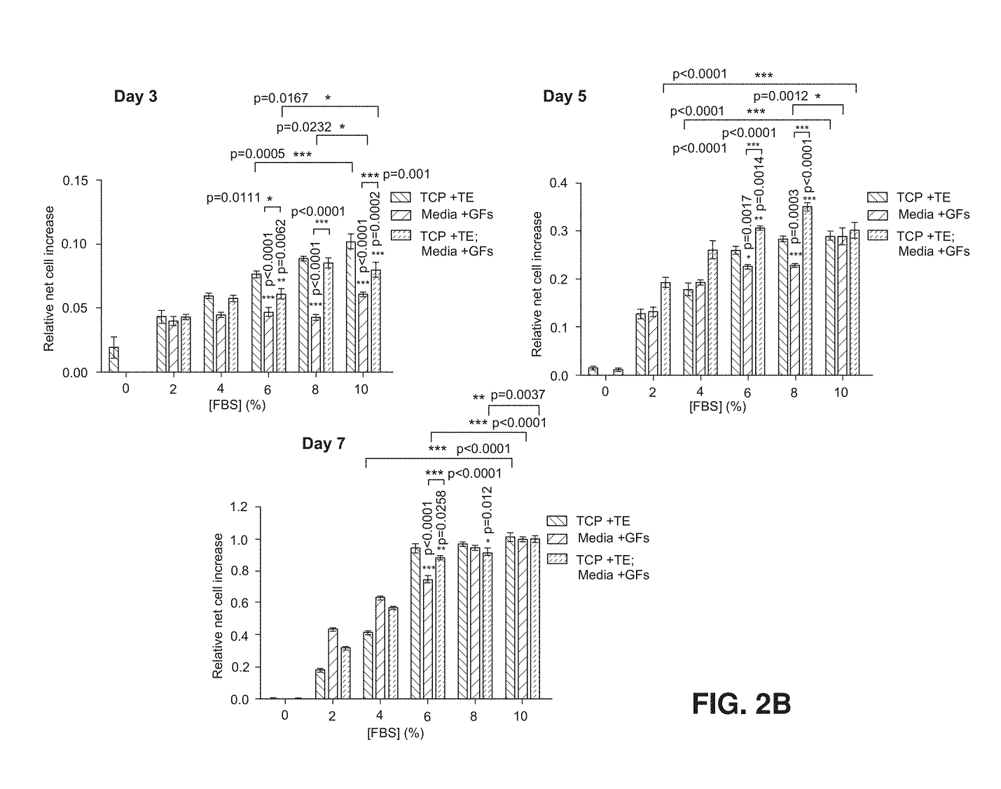

Due to the persistence of tropoelastin's pro-proliferative activity in media with 7% (v/v) FBS, the maximum extent of serum reduction that would not affect tropoelastin-mediated MSC expansion was investigated (FIG. 2A). Cells were grown in decreasing amounts of FBS (0-10% (v/v) in media) on TCP and on TCP coated with tropoelastin or fibronectin. Serum reduction was well-tolerated by MSCs during the early stages of proliferation. Until 3 days post-seeding, MSC numbers significantly decreased on bare or fibronectin-coated surfaces only when serum was completely absent in media, and on tropoelastin-coated surfaces when serum was reduced by 80%. Subsequently, however, MSC numbers on bare or fibronectin-coated surfaces progressively declined with greater serum reduction. After 7 days, MSC proliferation on TCP or fibronectin decreased by 27±1% and 15±0.1%, respectively, following a mere 20% reduction in serum. In contrast, cell expansion on a tropoelastin substrate remained unaffected by up to a 40% decrease in serum. At this serum concentration, MSC proliferation on bare and fibronectin-coated TCP was inhibited by 35±1% and 25±1%, respectively, compared to that in normal media.

While fibronectin and tropoelastin equally promoted MSC propagation in full serum media, the benefits of fibronectin were significantly diminished upon serum reduction. At these lower serum concentrations, i.e. 2-8% (v/v) of the media composition, tropoelastin-coated surfaces consistently and significantly enhanced MSC proliferation compared to bare or fibronectin-coated surfaces by 135±5 to 309±12% and 76±4 to 86±6%, respectively. These findings strongly indicate that tropoelastin can uniquely compensate for substantial serum reduction in media without compromising MSC expansion levels.

### Example 4

### Tropoelastin Allows for Greater Serum Reduction Compared to Growth Factors

The ability to promote high levels of stem cell growth in low serum conditions, as demonstrated by tropoelastin, is a property typically ascribed to growth factors. On this basis, this functionality of substrate-bound tropoelastin with that of IGF-1 and bFGF was compared (FIG. 2B). Consistent with previous observations, MSCs were more susceptible to the effects of serum reduction during the later proliferative stages. By 7 days post-seeding, cell numbers on TCP in growth factor-containing media was unaltered in 8% (v/v) FBS, indicating that the combined presence of IGF-1 and bFGF allows for slight (20%) serum reduction during culture. In 6% (v/v) FBS, however, cell numbers in media with growth factors were significantly decreased by 25±2% compared to those in full serum media. In contrast, cells on tropoelastin in the absence of growth factors sustained uncompromised levels of proliferation following a 40% decrease in serum. At this serum concentration, tropoelastin improved MSC proliferation by 23±3% compared to IGF-1 and bFGF in tandem, indicating that tropoelastin is functionally superior to the growth factors in stimulating MSC expansion in substantially reduced serum conditions.

Interestingly, surface-bound tropoelastin similarly allowed for 40% serum reduction when growth factors were also present in media, but only until 5 days post-seeding. After this time point, cells tolerated a maximum of 20% serum reduction without deleterious effects to proliferation. These results implicate the possibility of alternative pathways involved in serum compensation, which may depend on cell exposure to the soluble growth factors relative to the substrate-bound tropoelastin.

### Example 5

### Tropoelastin in Solution Promotes MSC Proliferation Similarly to Surface-Bound Tropoelastin

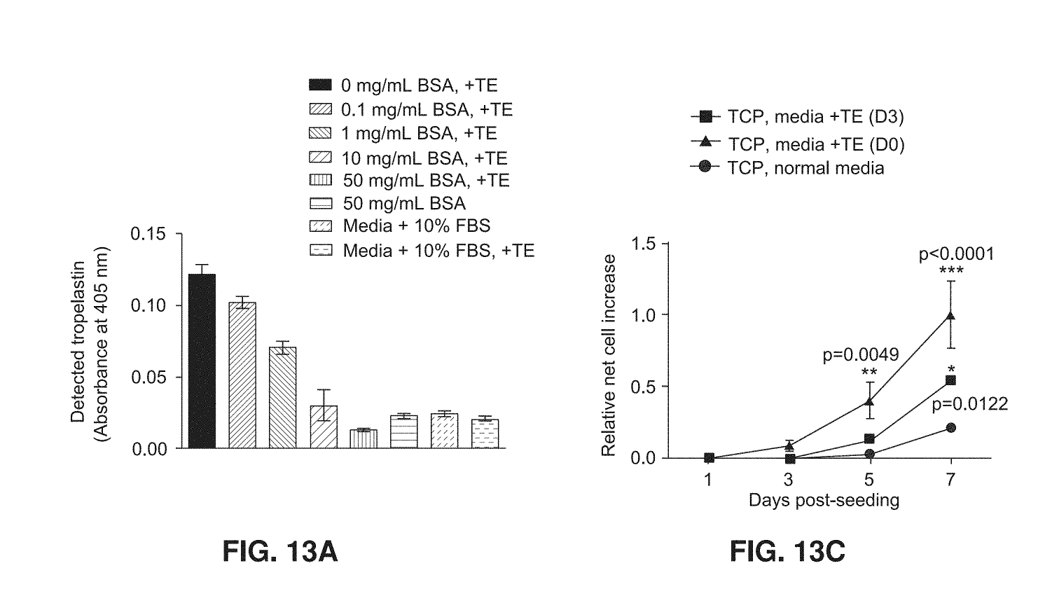

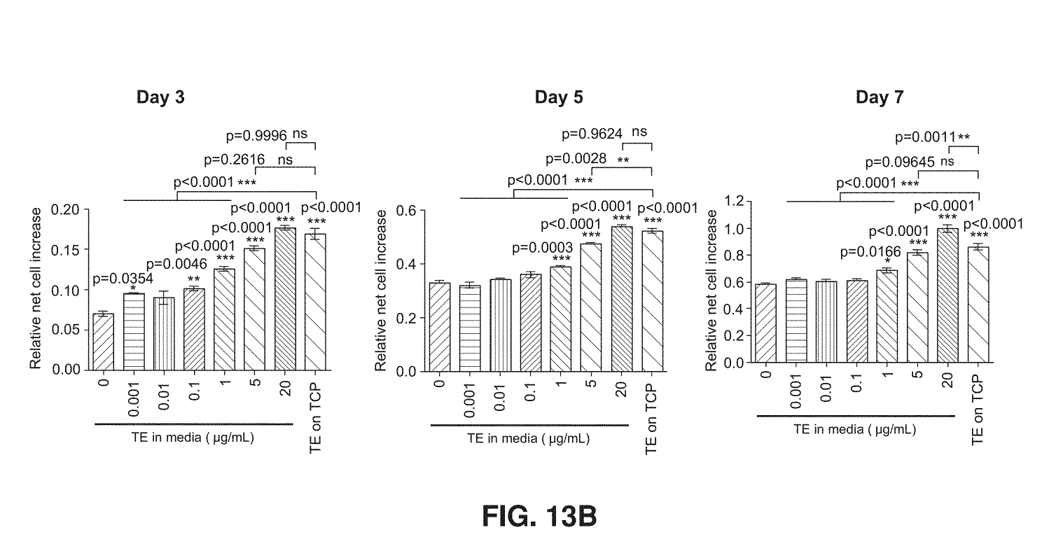

To determine whether the mitogenic activity of tropoelastin is conditional upon its immobilization to the culture substrate and the provision of mechanical cues, it was tested whether tropoelastin in solution achieves the same cell expansion benefits as the surface-bound protein. When tropoelastin was added to tissue culture wells that have been pre-incubated with normal media, the protein did not adhere to the well surface and remained in solution, most likely due to surface blocking by serum proteins such as albumin (FIG. 13A).

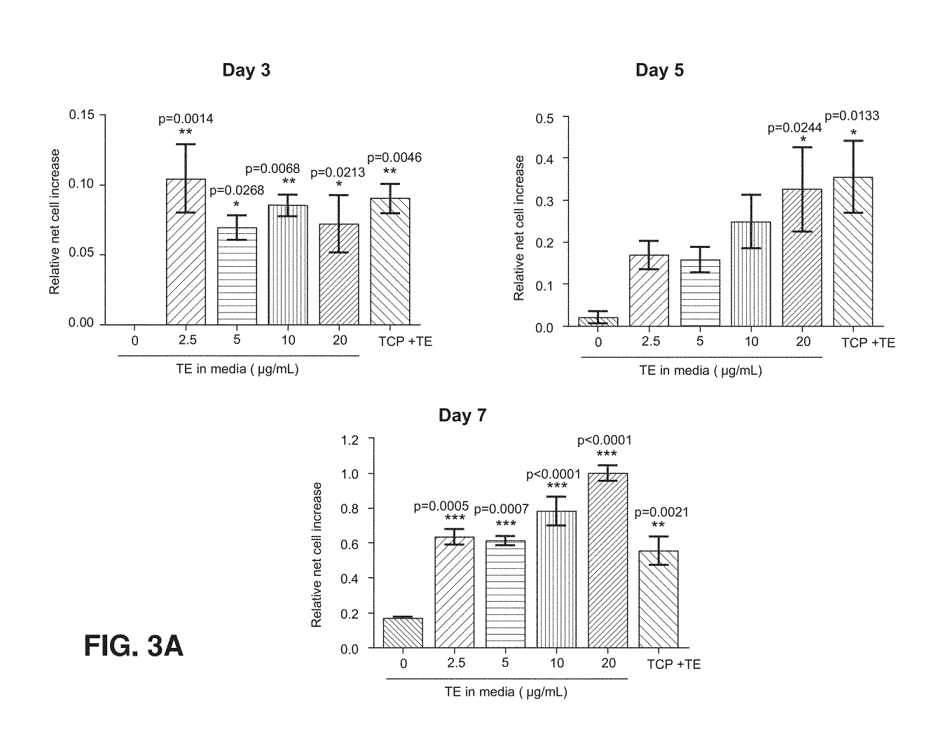

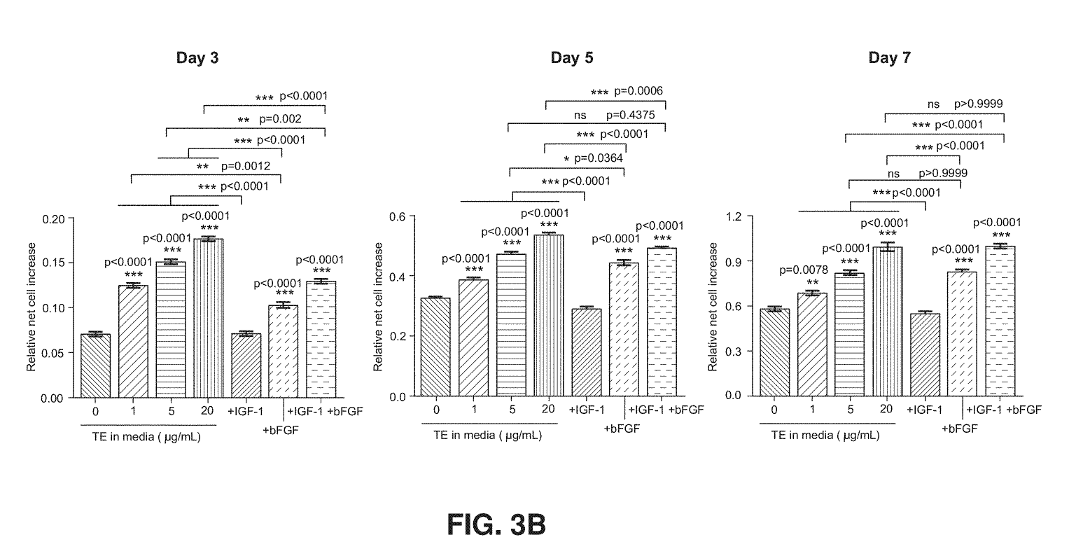

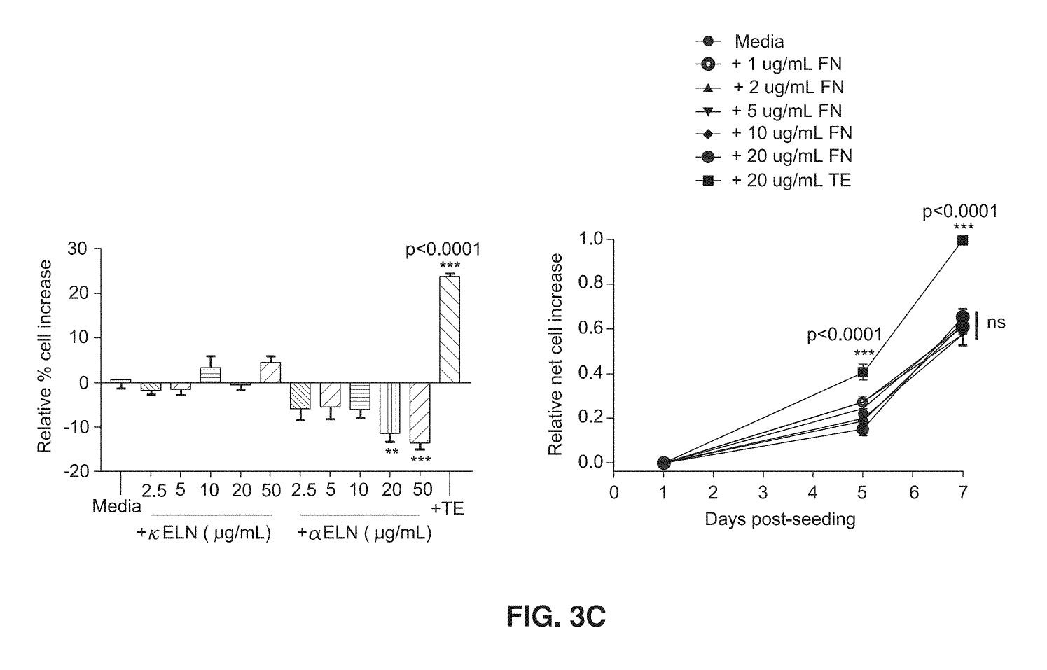

Soluble tropoelastin at concentrations as low as 1 μg/mL consistently promoted MSC proliferation over 7 days compared to normal media (FIG. 13B). However, tropoelastin concentrations of at least 2.5 μg/mL were required to stimulate MSC proliferation to a comparable extent as substrate-bound tropoelastin (FIG. 3A). This concentration represents a similar amount of protein expected to adhere during substrate coating with excess (20 μg/mL) tropoelastin. Increasing the solution concentration of tropoelastin to 20 μg/mL further improved MSC proliferation by 80±8% over substrate-bound tropoelastin at 7 days post-seeding. These results demonstrate that tropoelastin above a threshold concentration in solution significantly promotes MSC proliferation. Supplementation of media with tropoelastin is at least functionally equivalent to coating the culture substrate with tropoelastin and allows temporal control of the associated increase in proliferation levels (FIG. 13C). Evidently, tropoelastin can function as a signaling molecule in solution, similarly to growth factors, to actively enhance MSC expansion.

### Example 6

### Tropoelastin in Solution can Replace IGF-1 and bFGF in Full Serum Media

It was further investigated whether tropoelastin in solution, like substrate-bound tropoelastin, can mirror the effects of growth factors in eliciting a proliferative response from MSCs (FIG. 3B). It was previously observed that substrate-bound tropoelastin can replace either IGF-1 or bFGF in full-serum media. Media supplementation with IGF-1 alone did not increase MSC numbers compared to normal media. As such, tropoelastin in solution at or above 1 μg/mL triggered significantly elevated cell proliferation over 7 days compared to normal media or media with IGF-1. This level of increase is dose-dependent, ranging from 18±5% with 1 μg/mL tropoelastin to 69±7% with 20 μg/mL tropoelastin.

Soluble tropoelastin can likewise replace bFGF in media. During early-stage proliferation (3 days post-seeding), soluble tropoelastin at and above 1 μg/mL surpassed bFGF by up to 74±2% in promoting MSC expansion. At later time points until 7 days post-seeding, soluble tropoelastin at 5 μg/mL was comparable to bFGF; and at 20 μg/mL was 18±5% more potent than bFGF for MSC propagation.

Furthermore, while substrate-bound tropoelastin was functionally inferior to the cumulative benefit of IGF-1 and bFGF in full serum media, soluble tropoelastin at 20 μg/mL supported MSC expansion equivalent to that in media containing both growth factors. These findings illustrate that tropoelastin in solution closely reflects the pro-proliferative capability of growth factors. At 5 μg/mL, tropoelastin can replace either IGF-1 or bFGF, while a higher concentration of 20 μg/mL can adequately replace both growth factors without loss of MSC proliferative potential.

### Example 7

### Soluble Elastin Fragments or Fibronectin Do Not Promote MSC Proliferation

To determine whether the potent mitogenic ability of tropoelastin in solution is similarly captured within fragments of the cross-linked protein, cells were grown in normal media, in tropoelastin-supplemented media, or in media containing increasing amounts of soluble κ-elastin (κELN) or α-elastin (αELN), which are peptides obtained from partial base or acid hydrolysis of native elastin (FIG. 3C). Neither κELN nor αELN stimulated MSC proliferation above that in normal media. On the contrary, higher concentrations of αELN at 20-50 μg/mL suppressed cell expansion by up to 14±1%. Clearly, the pro-proliferative effect of tropoelastin in solution requires the intact, full-length molecule.

This ability of tropoelastin to propagate cells in solution is unique for a matrix protein. Fibronectin promoted MSC expansion when coated on the substratum at concentrations as low as 2 μg/mL but did not trigger any proliferative response when present in solution at up to 20 μg/mL (FIG. 3D). These results emphasize the singularity of tropoelastin's dual capacity for modulating MSC proliferation, as an underlying substrate and as a soluble factor.

### Example 8

### MSCs Retain Cell Phenotype During Tropoelastin-Mediated Expansion

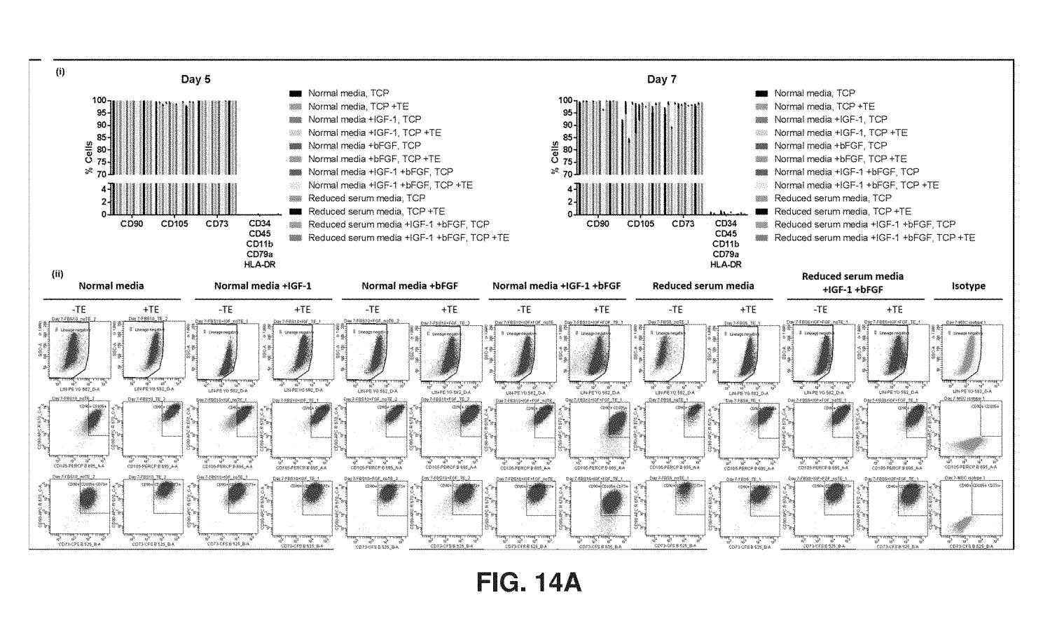

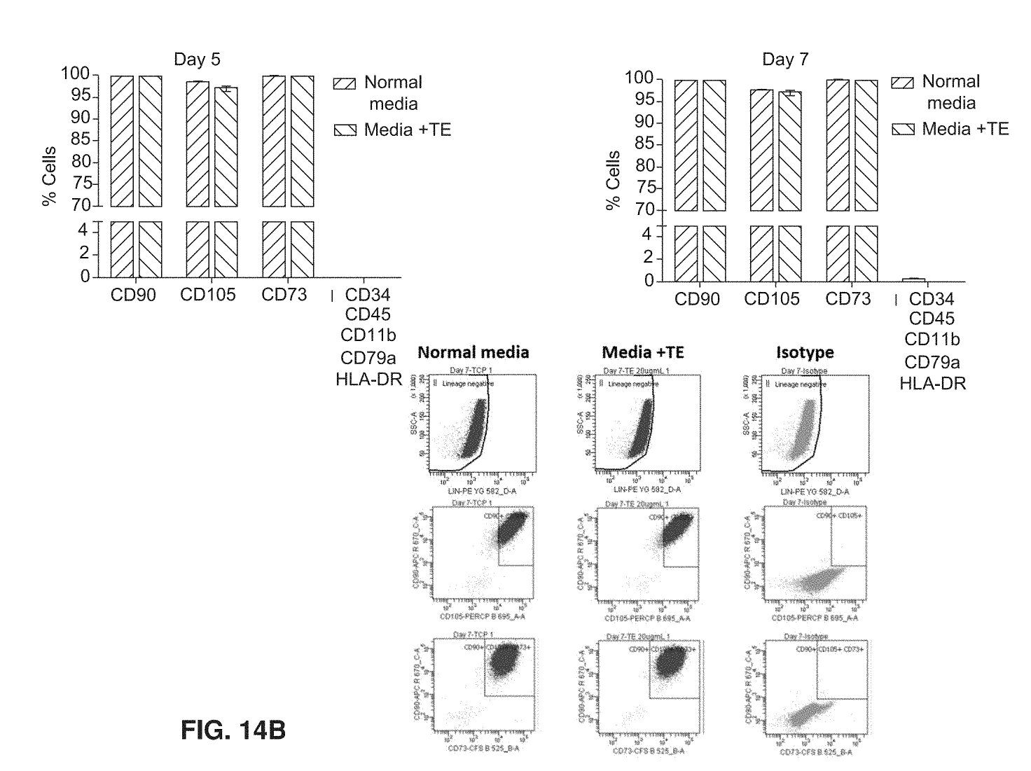

An essential consideration when inducing MSC expansion is the maintenance of the native stem cell phenotype. Flow cytometry analyses indicated that cells cultured for 5 or 7 days on tropoelastin-coated surfaces, in full or reduced serum media with and without growth factors, exhibited characteristic MSC marker profiles (FIG. 14A). At 5 days post-seeding, more than 95% of cells in all media formulations expressed the positive MSC markers CD90, CD105 and CD73, while more than 98% lacked expression of hematopoietic stem cell markers CD34, CD45, CD11b, CD79a and HLA-DR, in accordance with the MSC identification criteria set by the International Society for Cellular Therapy.

At 7 days post-seeding, a decreased proportion of cells expressed all three MSC markers when grown on bare TCP in media containing only IGF-1 or bFGF. Only 83.9±0.7% of cells in IGF-1 supplemented media, and 92.9±5.9% of cells in bFGF supplemented media were positive for CD105. Likewise, only 89.1±0.1% of cells in IGF-1 containing media expressed CD73. These results point to the combined role of IGF-1 and bFGF in maintaining MSC phenotype during longer-term cell expansion.

Remarkably, substrate coating with tropoelastin restored the MSC marker expression levels of cells in these sub-optimal media preparations to requisite thresholds. MSC phenotype was fully retained in all instances where substrate-bound tropoelastin was used to replace one or both growth factors in full serum or reduced serum media. Similarly, cells grown in media containing 20 μg/mL soluble tropoelastin also displayed characteristic CD90+, CD105+, CD73+ and lineage negative expression profiles (FIG. 14B).

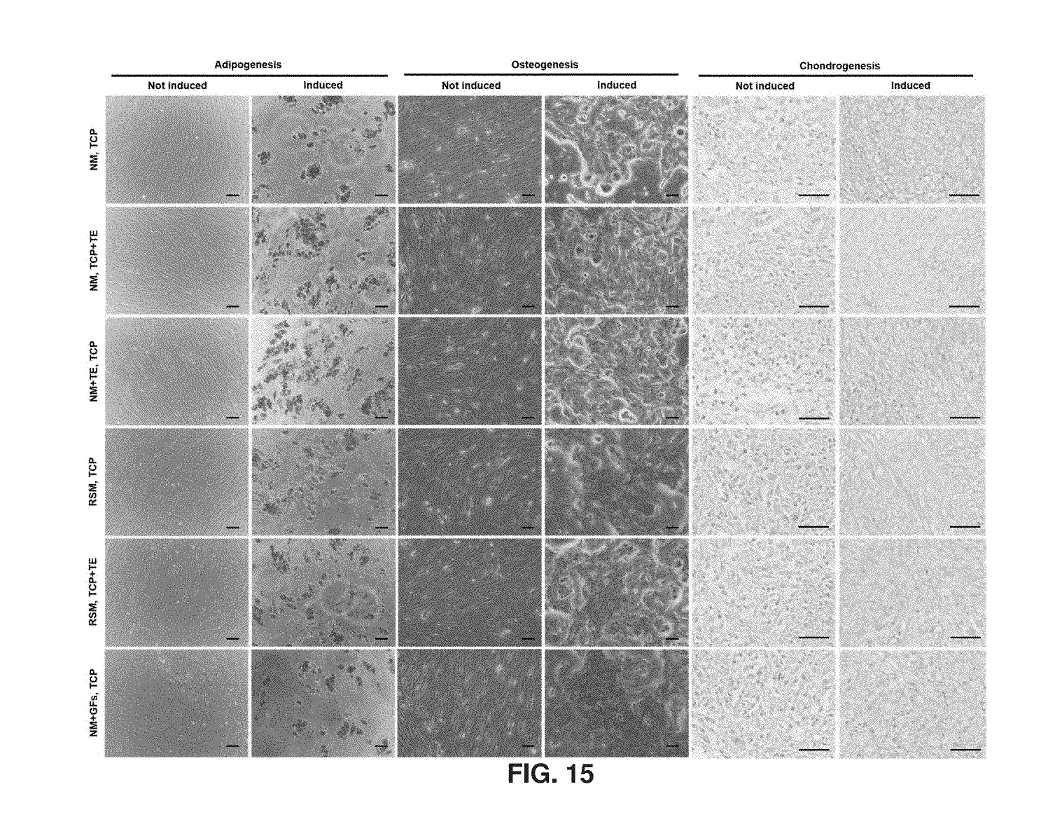

Concomitant with the retention of cell surface markers, MSCs expanded in the presence of substrate-bound or soluble tropoelastin, as a replacement for growth factors in normal or reduced serum media, also exhibited the capacity for multi-lineage differentiation (FIG. 15). When induced with adipogenic media, these MSCs developed characteristic intracellular lipid droplets that appeared bright red with Oil Red O staining. When induced with osteogenic media, they formed mineralized calcium deposits visualized as red nodules by Alizarin Red S staining. When induced with chondrogenic media in micromass pellet culture, MSCs showed glycosaminoglycan-rich regions stained blue-green by Alcian Blue, which were indicative of cartilage formation. These histological features were absent in non-induced samples. Taken together, these findings strongly support the ability of tropoelastin to preserve MSC phenotype and multipotent behavior throughout the amplified expansion process.

### Example 9

### Tropoelastin Modulates MSC Attachment and Spreading via αv Integrins

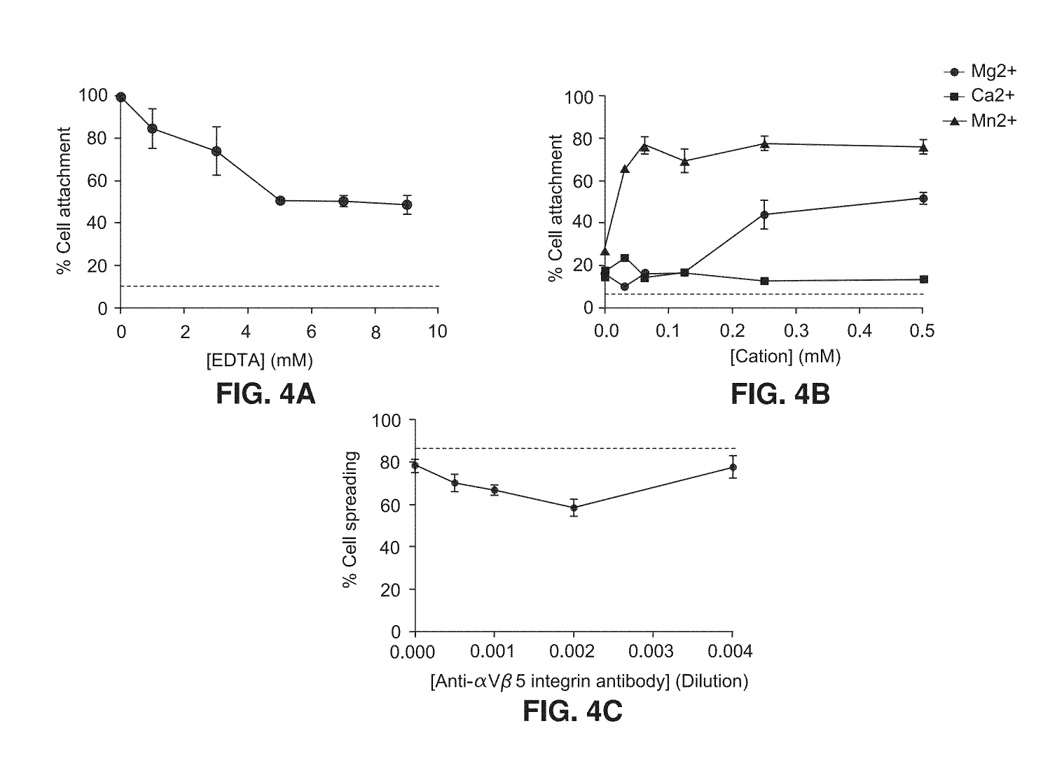

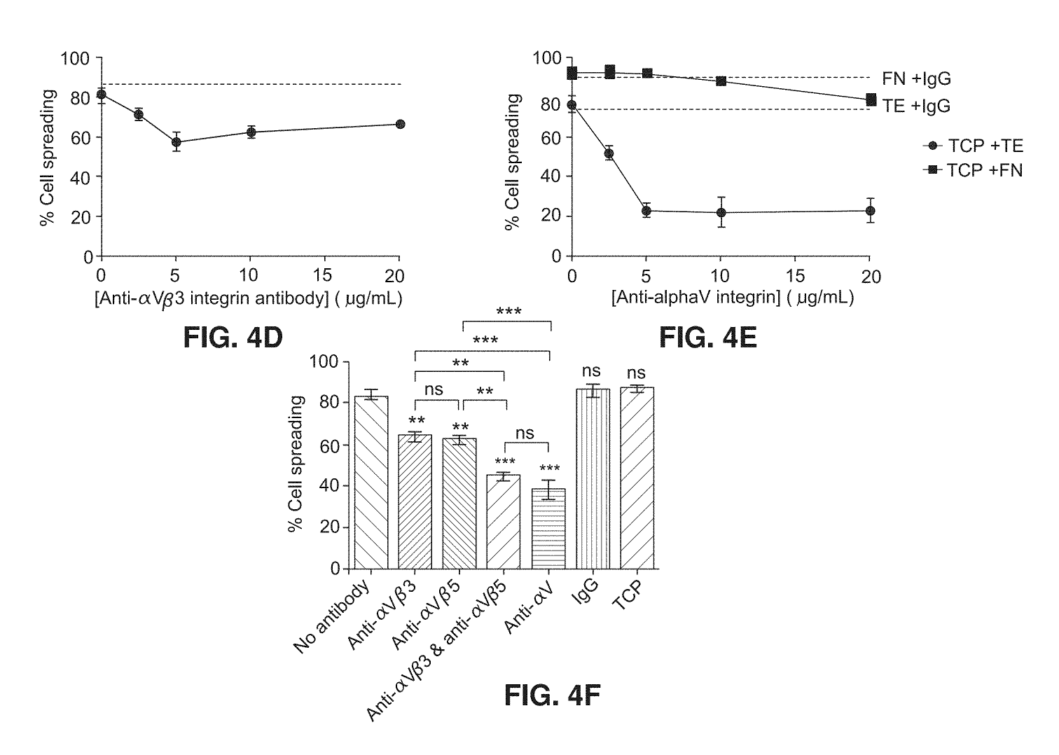

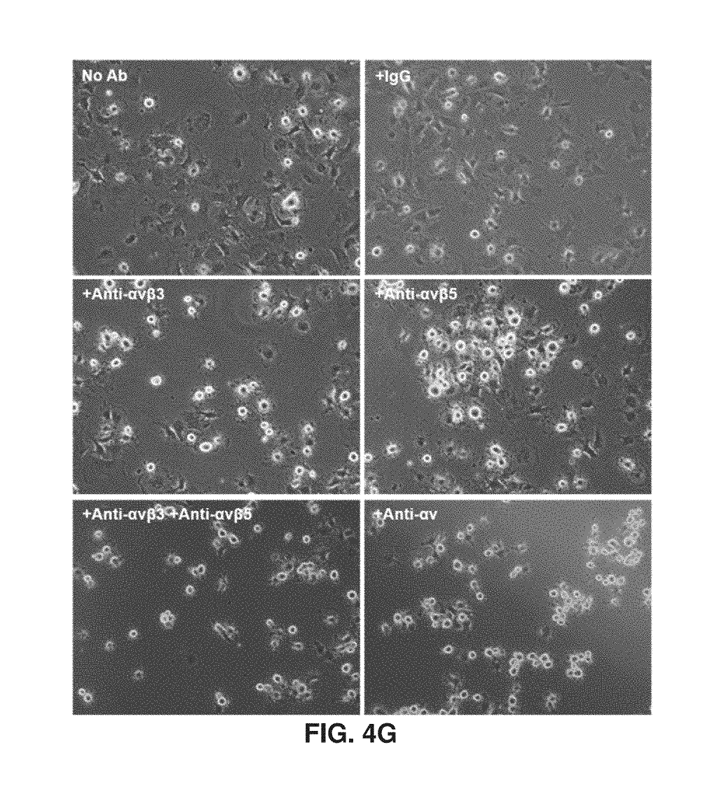

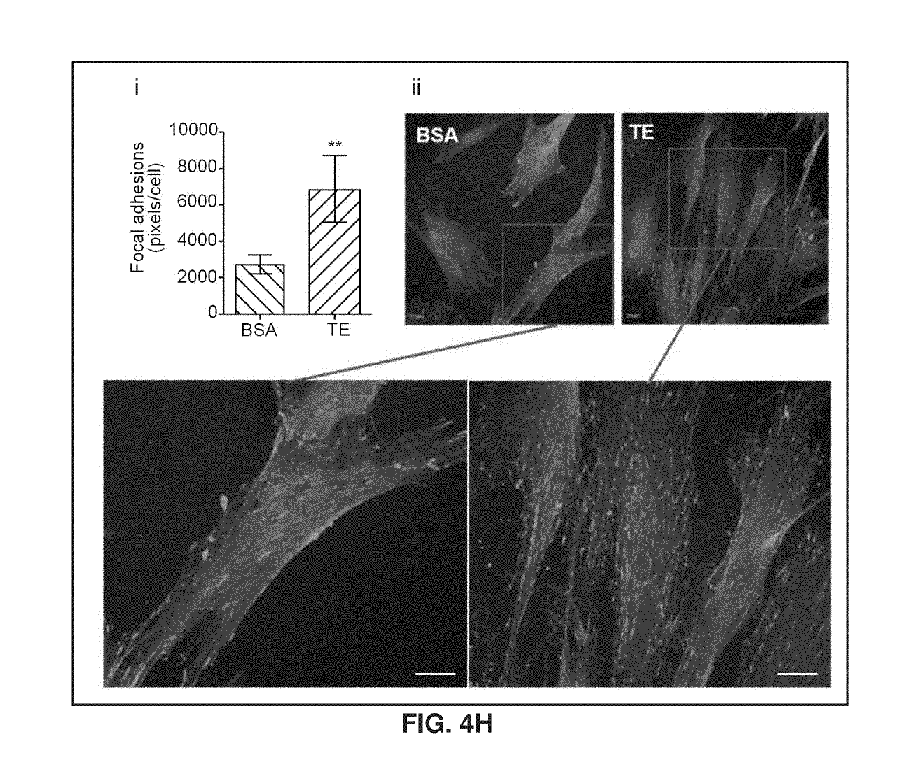

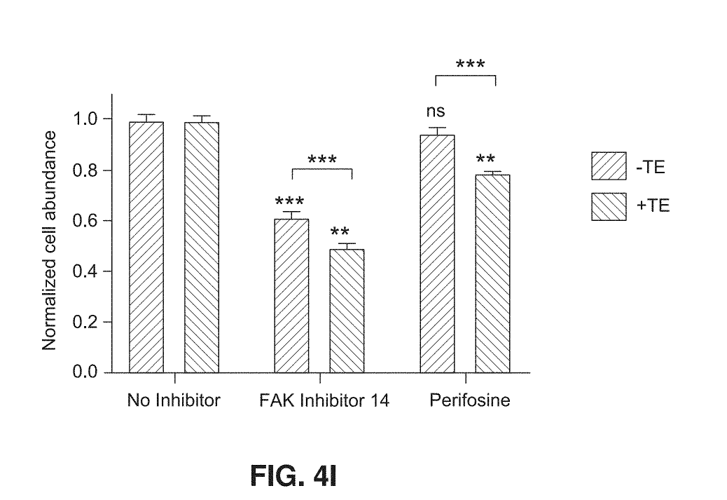

To determine the involvement of integrin receptors in tropoelastin modulation of MSC behavior, the divalent cation dependence of tropoelastin-MSC interaction were analyzed. Addition of the chelator EDTA significantly inhibited MSC attachment to substrate-bound tropoelastin in a dose-dependent manner (FIG. 4A). In the presence of 5 mM EDTA, MSC binding to tropoelastin was maximally reduced by 48.9±0.5%. Furthermore, MSCs displayed minimal (20.0±2.1%) adhesion to tropoelastin in a cation-free environment (FIG. 4B). The subsequent addition of up to 0.5 mM Ca²⁺ did not improve MSC binding (13.7±1.0%); Mg²⁺ promoted moderate (51.8±2.6%) cell attachment, while Mn²⁺ restored (76.1±3.2%) cell adhesion to tropoelastin. This selective cation dependence is characteristic of an integrin-mediated cell binding mechanism.

As further confirmation of the role of integrins in MSC interactions with tropoelastin, specific integrin-blocking antibodies impeded MSC spreading on a tropoelastin substrate (FIGS. 4C-4G). The anti-αvβ5 and anti-αvβ3 integrin antibodies inhibited cell spreading on tropoelastin in a dose-dependent manner until optimal blocking concentrations were reached (FIG. 4C-D). This inhibition was heightened with a pan anti-αv integrin subunit antibody (FIG. 4E). Antibody specificity was validated by the minimally inhibited spreading on fibronectin (78.8±2.3%), which is known to alternatively interface with α5 and αv integrins, compared to the no antibody (92.5±2.6%) or IgG (90.1%) controls. At optimal antibody concentrations, the anti-αvβ5 and anti-αvβ3 antibodies significantly decreased MSC spreading on tropoelastin by 24.9±2.7% and 22.7±2.8%, respectively (FIG. 4F). The combined addition of anti-αvβ5 and anti-αvβ3 further inhibited spreading by 46.0±2.5%, which was similar to the 53.6±5.6% inhibition by the anti-αv antibody. Cell spreading on tropoelastin was unaffected by a non-specific IgG antibody, or in the absence of antibodies. Representative images of MSCs seeded on tropoelastin showed that in the absence of integrin-blocking antibodies, majority of cells possessed a spread morphology characterized by a flattened, phase-dark cell body (FIG. 4G). In contrast, in the presence of anti-integrin antibodies, a markedly higher proportion of cells appeared unspread with a rounded, phase-bright morphology. In addition, vinculin staining of substrate-bound MSCs revealed a number of dot-like focal complexes and streak-like focal adhesions at the cell center and periphery. Cells adhered to tropoelastin possessed 1.5±0.7 fold increased focal adhesions per cell compared to those on bovine serum albumin (BSA) (FIG. 4H). Taken together, these results support the role of αv integrins in mediating MSC interactions with tropoelastin.

### Example 10

### Tropoelastin Modulates MSC Expansion via αv Integrins

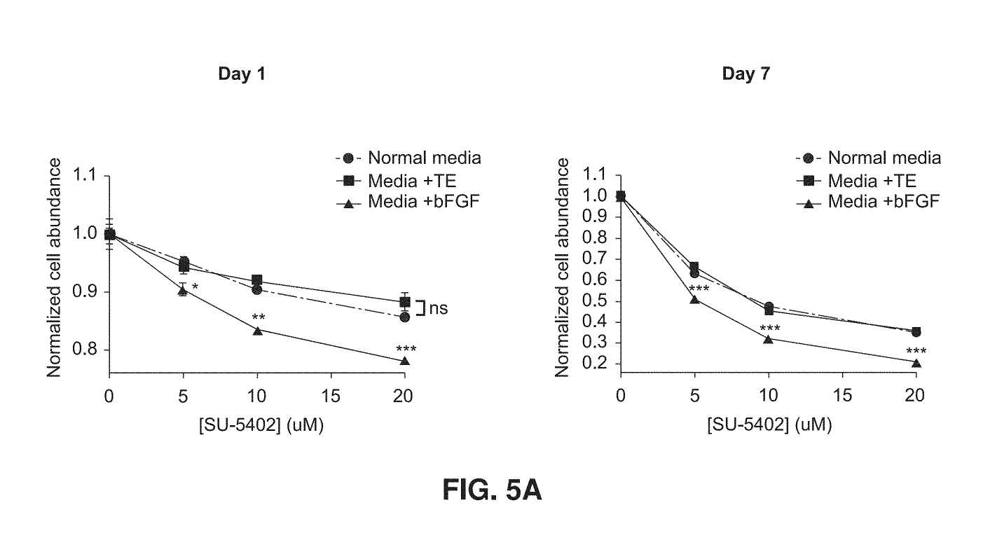

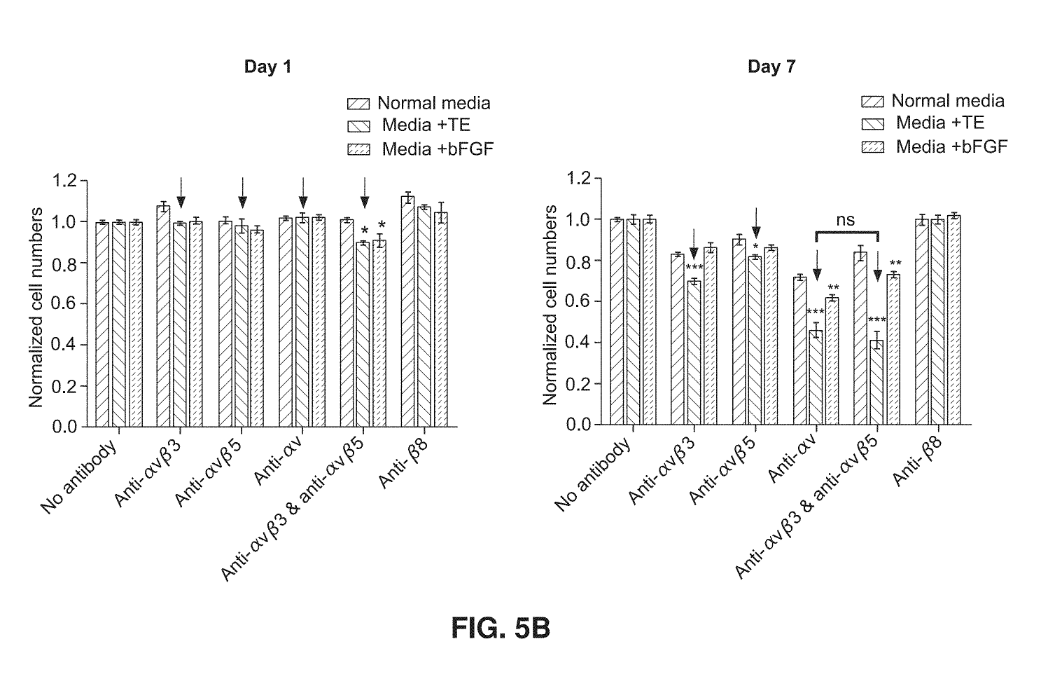

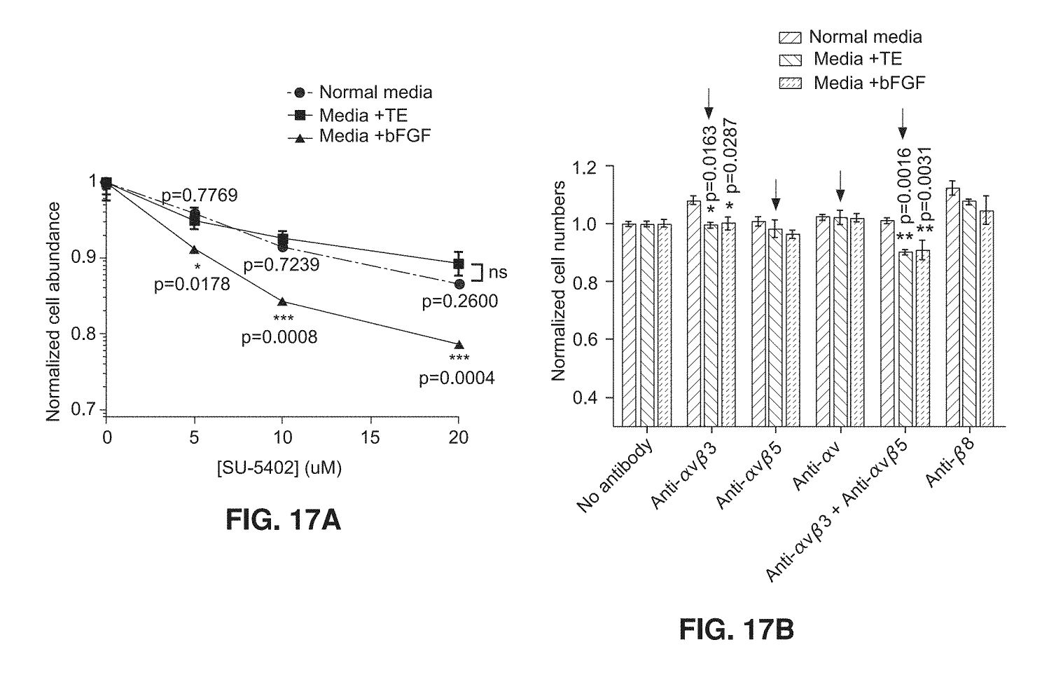

It was discovered that soluble tropoelastin-mediated MSC expansion is attenuated by integrin blocking but not by growth factor receptor inhibition. The proliferative advantages of growth factors were primarily attributed to bFGF rather than IGF-1; therefore, bFGF was selected as the functional parallel to tropoelastin. The addition of SU-5402, a fibroblast growth factor receptor (FGFR) inhibitor, hindered MSC proliferation over 7 days in a dose- and time-dependent manner (FIGS. 5A and 17A). The extent of inhibition varied significantly among cells cultured in normal media, media containing bFGF, or media containing tropoelastin in solution. The most profound inhibition, up to a 78.9±0.7% reduction in overall cell numbers compared to the no inhibitor control, consistently occurred with cells grown in bFGF-supplemented media. In contrast, the reduced cell proliferation in tropoelastin-supplemented media was similar to that in normal media and can likely be ascribed to the non-specific effects of SU-5402. These results suggest that, unlike bFGF, soluble tropoelastin stimulates MSC propagation via an FGFR-independent pathway.

The cell proliferative consequences of blocking integrin receptors, specifically αvβ3, αvβ5 or all αv subunit integrins, over 7 days was also explored (FIGS. 5B and 17B). Antibody inhibition of αv integrin activity universally diminished MSC proliferation to varying degrees, regardless of culture media composition. However, the decrease in cell expansion was consistently greater for cells in tropoelastin-supplemented media than cells in normal media or in bFGF-supplemented media. Compared to the no antibody control, inclusion of the anti-αvβ3 or anti-αvβ5 antibody significantly inhibited tropoelastin-mediated MSC proliferation by 30±1.3% and 18.1±0.9%, respectively. Addition of both anti-αvβ3 and anti-αvβ5 antibodies decreased cell expansion by 58.9±4.2%, which was similar in magnitude to the 54.1±3.7% reduction in cell numbers by the pan anti-αv antibody. A control antibody against β8integrins, which are not expressed by MSCs, did not affect cell proliferation. These findings strongly indicate that tropoelastin in solution, similarly to the substrate-bound protein, interacts with MSCs via integrins. Furthermore, αv integrins, specifically αvβ3 and αvβ5 in tandem, are involved in the propagation of pro-proliferative signals from tropoelastin during MSC expansion. Accordingly, specific inhibition of downstream signaling molecules, that is, focal adhesion kinase (FAK) by FAK inhibitor 14 and protein kinase B (PKB/AKT) by perifosine, significantly reduced tropoelastin-mediated proliferation by 50.7±2.0% and 21.3±0.5%, respectively (FIG. 4I). This decrease is significantly more profound than that caused by the nonspecific effects of these inhibitors and confirms the role of the integrin-FAK-PKB/AKT pathway in transducing tropoelastin-activated mitogenic signals in MSCs.

Interestingly, MSC proliferation in bFGF-supplemented media was also negatively impacted by the presence of integrin-blocking antibodies, although not to the same extent as that observed in cultures with tropoelastin. Significant inhibition relative to cells in normal media occurred only in the presence of anti-αv, or both anti-αvβ3 and anti-αvβ5, antibodies. These results further suggest that bFGF-mediated MSC proliferation is at least also partially dependent on αv integrin signaling.

### Example 11

### Substrate-Bound and Soluble Tropoelastin Attract MSCs

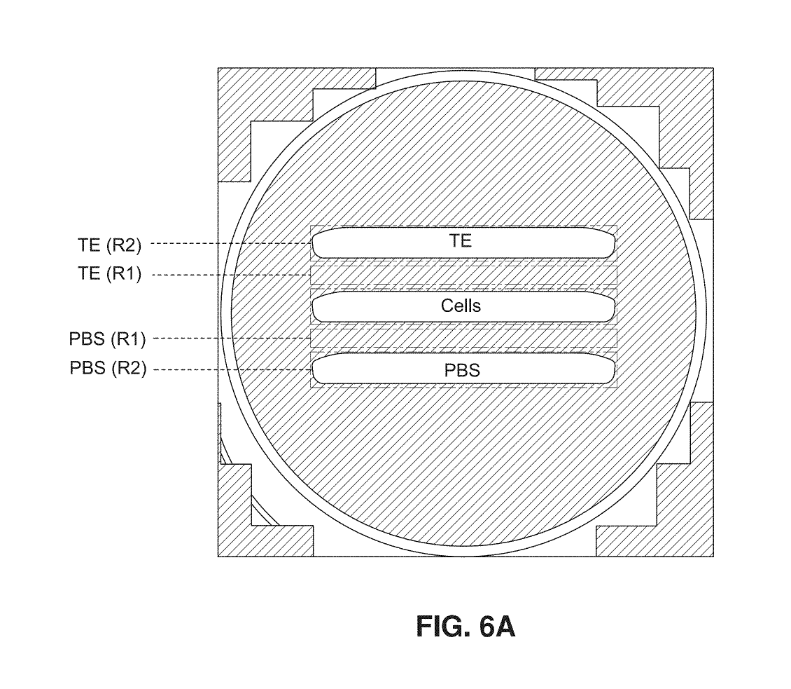

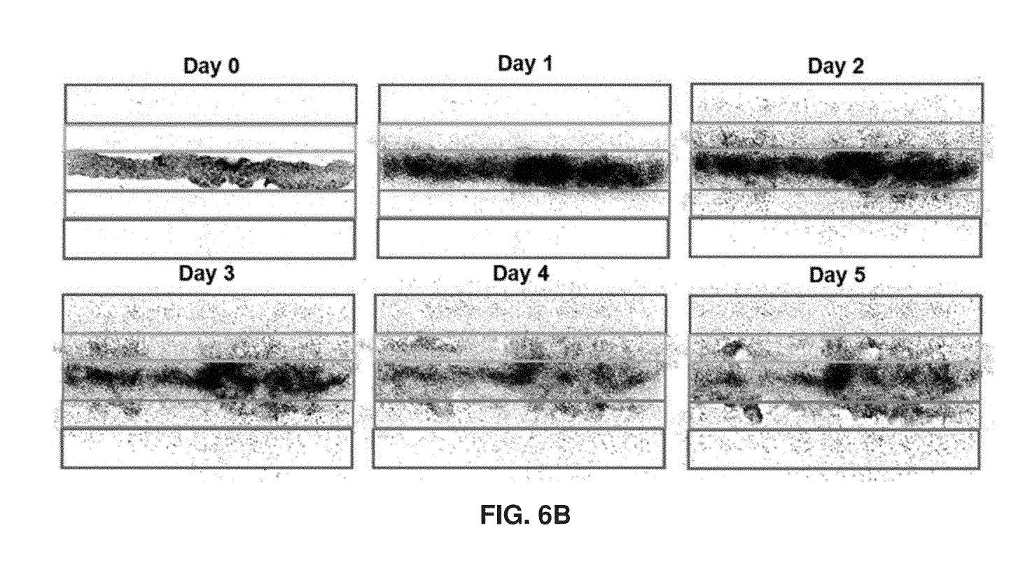

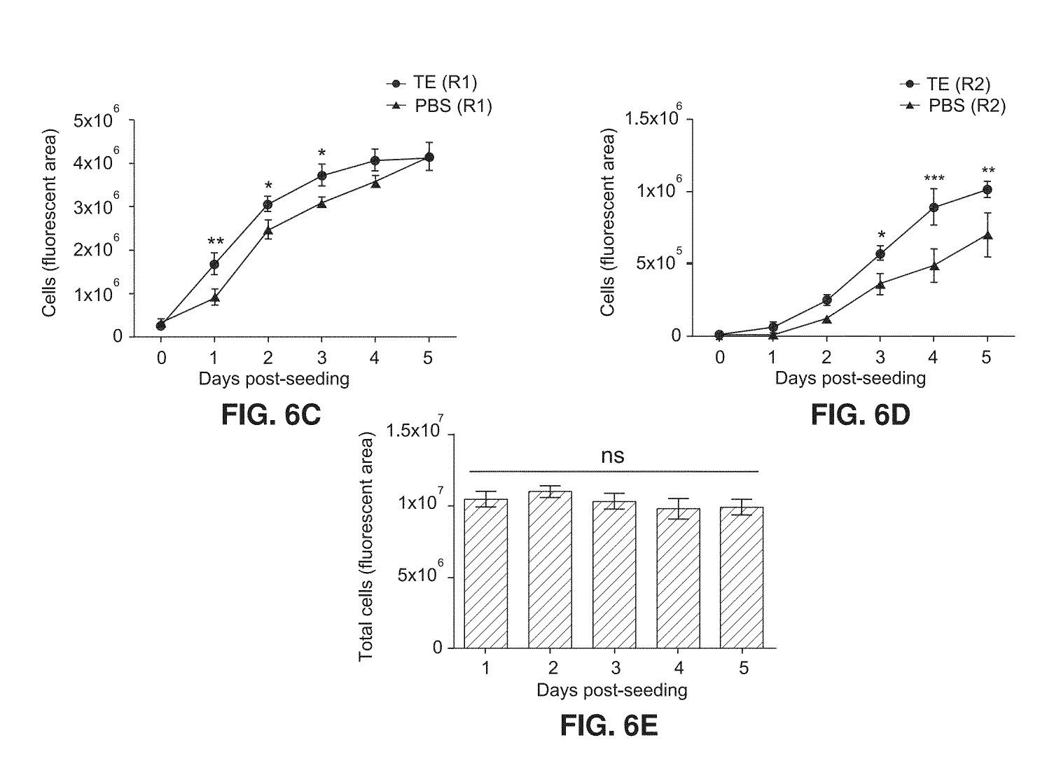

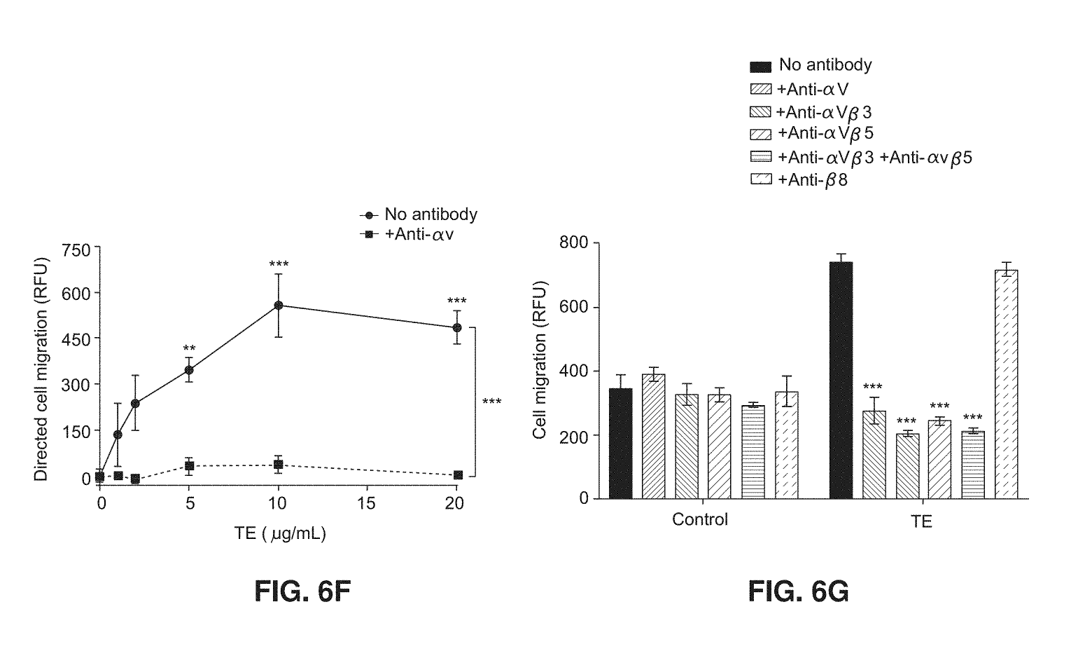

The potential of tropoelastin to attract MSCs, which would facilitate the tropoelastin-cell interactions for cell expansion was also investigated. Cells seeded in a central region were equidistantly flanked by regions optionally coated with tropoelastin (FIG. 6A). MSCs preferentially migrated towards the surface-bound tropoelastin compared to the no-protein control over 5 days (FIG. 6B). This haptotactic gravitation towards tropoelastin was manifested even at early time points (1-3 days post-seeding), in which the region between the cells and tropoelastin was significantly more populated than the corresponding region between the cells and the PBS control (FIG. 6C). By 5 days post-seeding, 45±8% more cells had migrated to the tropoelastin-coated region compared to the control (FIG. 6D). The higher cell abundance associated with tropoelastin was not due to the increased proliferation of migrated cells, as suggested by similar total cell numbers over the experimental period (FIG. 6E).

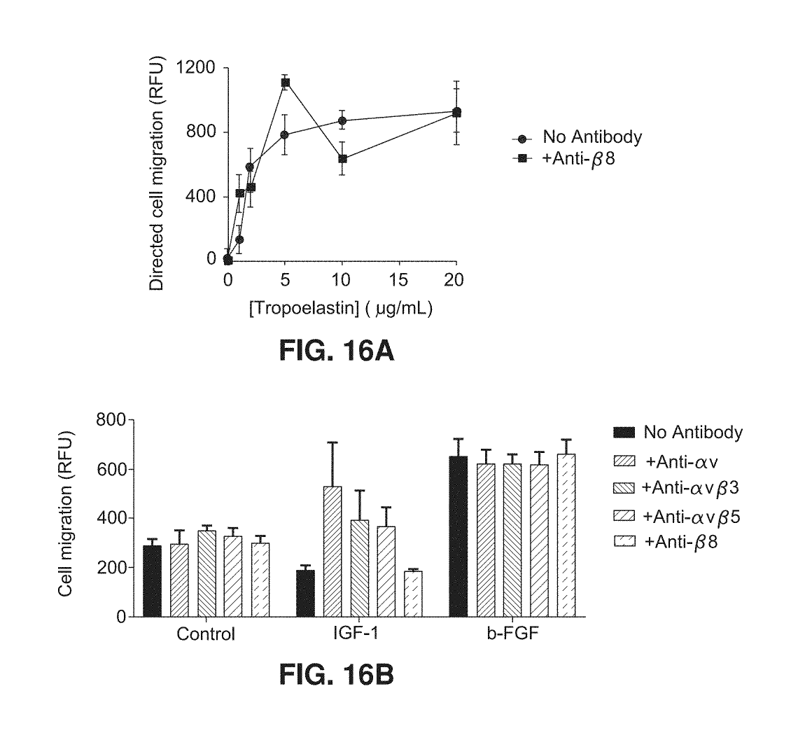

Similarly, MSCs also migrated towards a diffusible gradient of tropoelastin in a Boyden chamber set-up. Tropoelastin in solution induced a dose-dependent chemotactic response, which was abolished in the presence of the anti-αv integrin antibody (FIG. 6F). Antibodies that block all αv, either αvβ3 or αvβ5, or both αvβ3 and αvβ5 integrins effectively diminished tropoelastin-directed MSC migration to levels attributed to random cell mobility (FIG. 6G). In contrast, the control anti-β8 antibody did not affect MSC chemotaxis towards tropoelastin (FIG. 16A). Moreover, the αv-inhibitory antibodies did not alter levels of undirected cell migration in which no chemoattractant was present; nor did they inhibit chemotaxis towards IGF-1 or bFGF growth factors (FIG. 16B).

These results demonstrate the strong motogenic ability of substrate-bound and soluble tropoelastin, and the necessary and specific involvement of both αvβ3 and αvβ5 integrins in this process. This integrin dependence further implicates a method of MSC homing distinct from that used by chemotactic growth factors.

### Example 12

### Effect of Tropoelastin on MSC

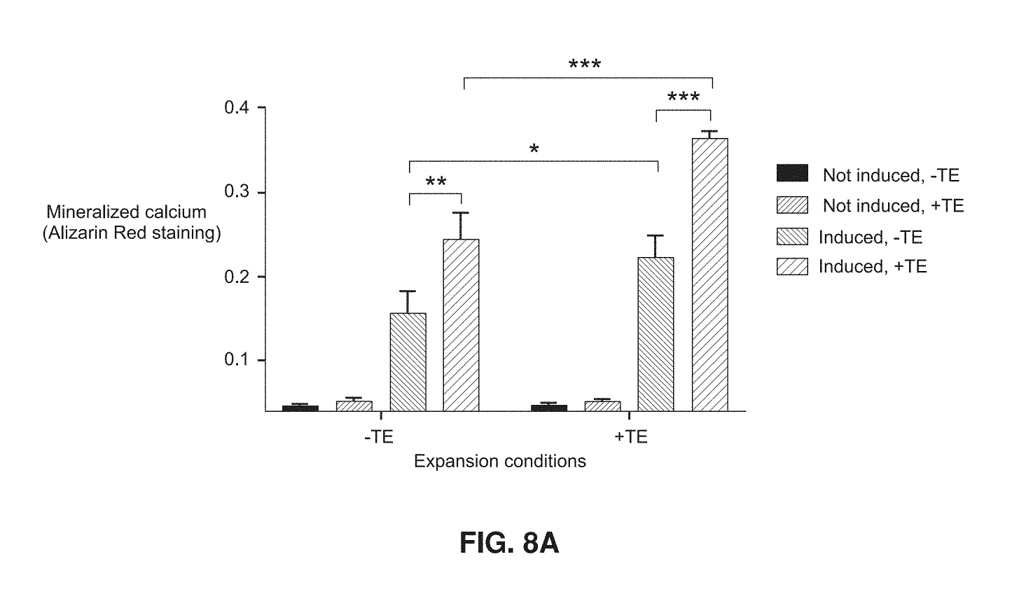

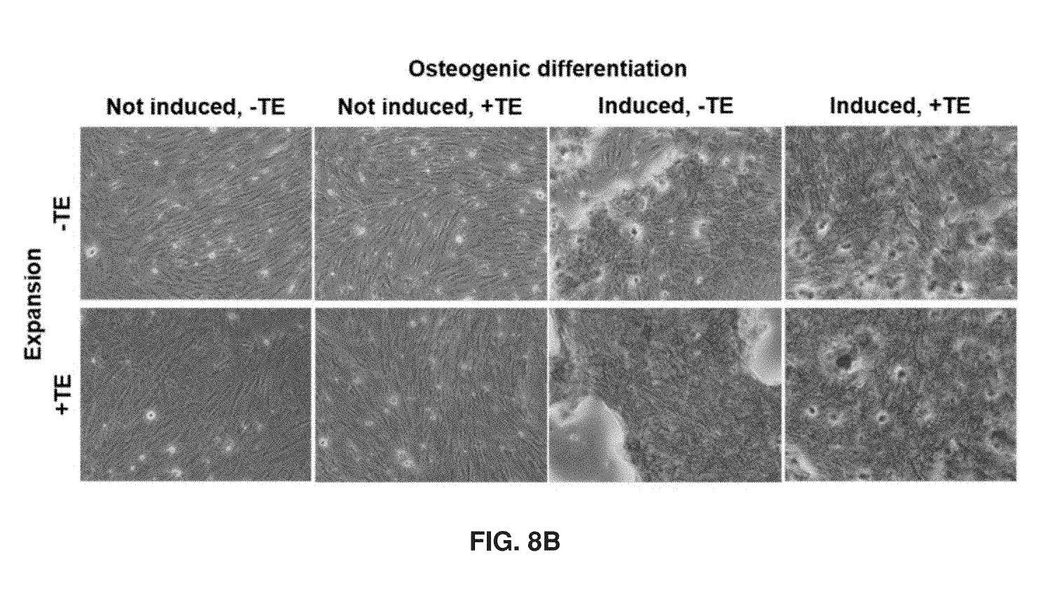

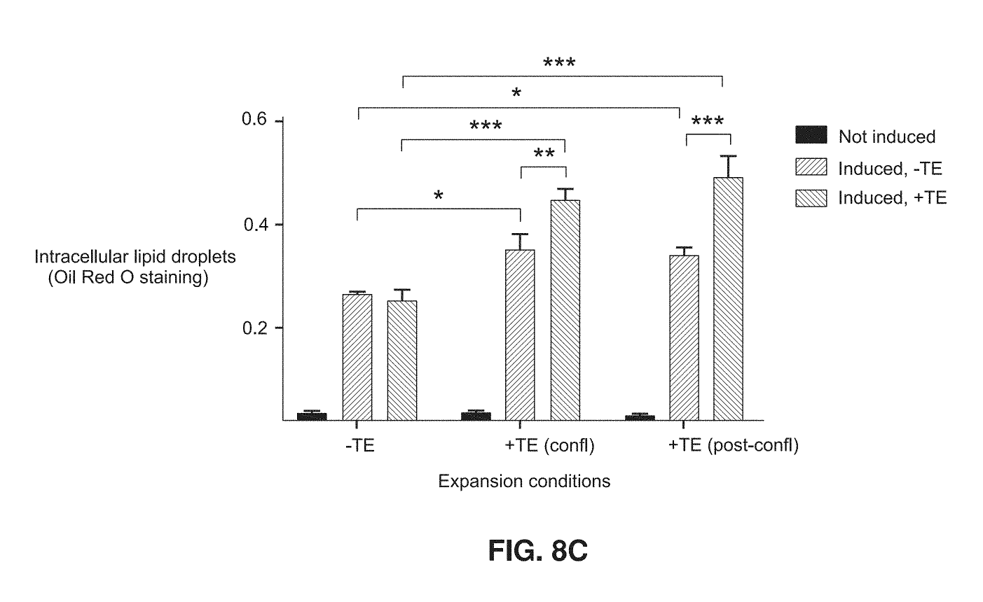

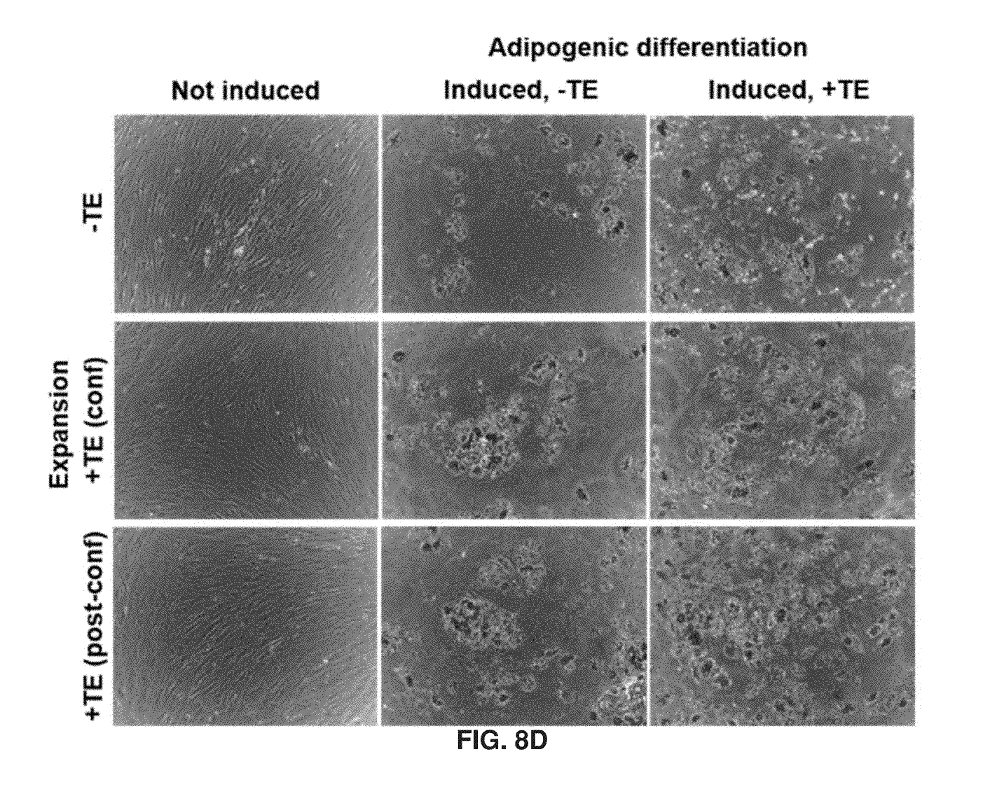

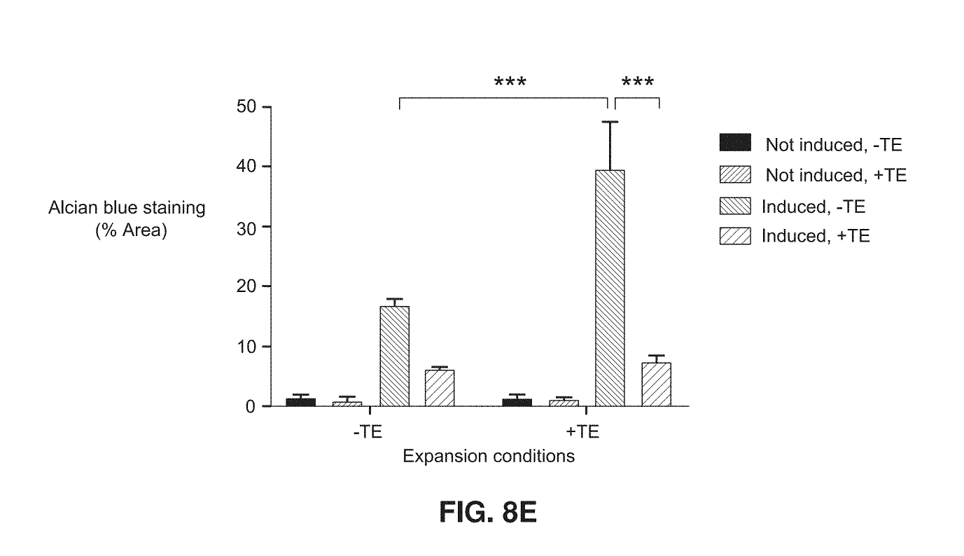

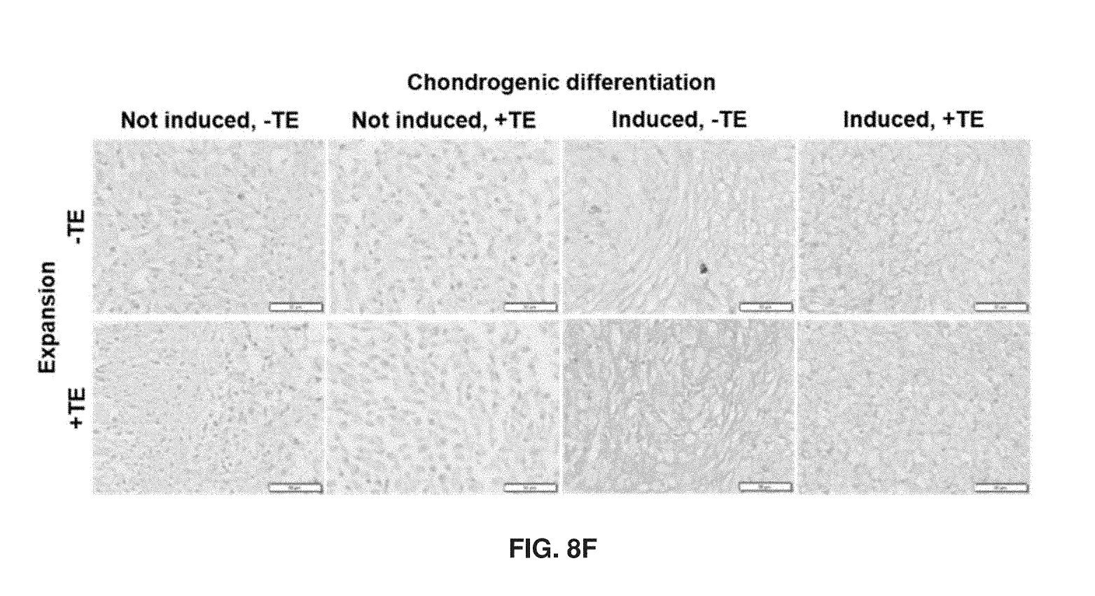

The effect of tropoelastin on MSC osteogenesis, adipogenesis and chondrogenesis was explored. As shown in FIG. 8A, the cells were grown in expansion conditions to study the effect in osteogenesis. The expansion conditions comprised growth media with and without tropoelastin. The increase in mineralized calcium is indicative of osteogenesis. As shown, cells that were induced and exposed to TE showed an increase in mineralized calcium concentrations. These results demonstrate the strong ability of soluble tropoelastin to induce osteogenesis as compared to the culture that lacked tropoelastin. FIG. 8B shows the ostegenic differentiation of the cells that were treated with TE as well. As shown, the osteogenic differentiation was shown in cells that were induced in the presence of TE.

FIGS. 8C and 8D demonstrate the effect of tropoelastin in adipogenic differentiation. As shown in FIG. 8C, cells that were induced to undergo adipogeneic differentiation exhibited an increase in intracellular lipid formation in the presence of TE as compared to the culture lacking TE.

FIGS. 8E and 8F demonstrate the effect of tropoelastin in chondrogenic differentiation. As shown in FIGS. 8E and 8F, cells that were expanded in the presence of TE exhibited increased glycosaminoglycan levels compared to cells that were expanded without TE, provided TE was not present during the differentiation stage. Addition of TE during the induction stage inhibited chondrogenic differentiation.

### Example 13

### Dose Response of Tropoelastin on MSC

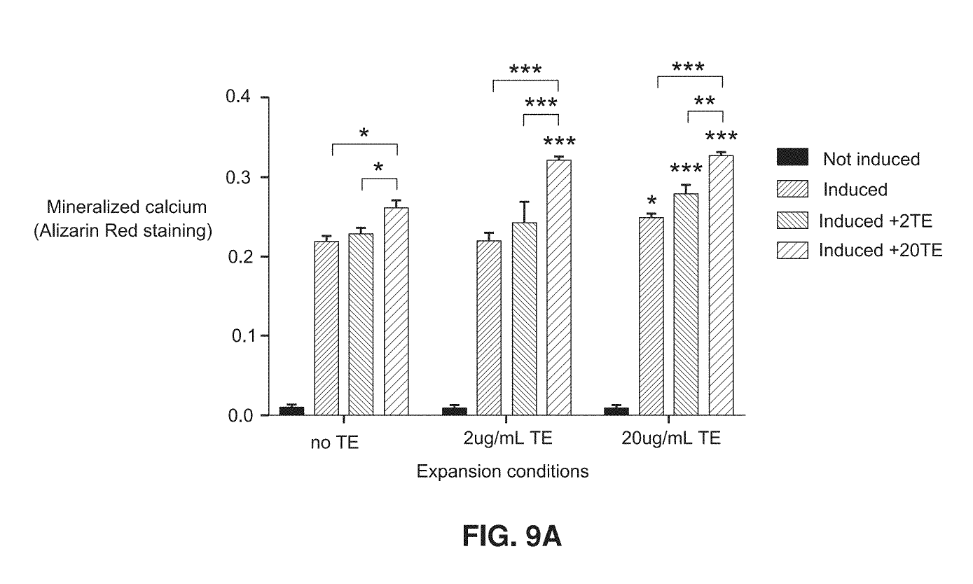

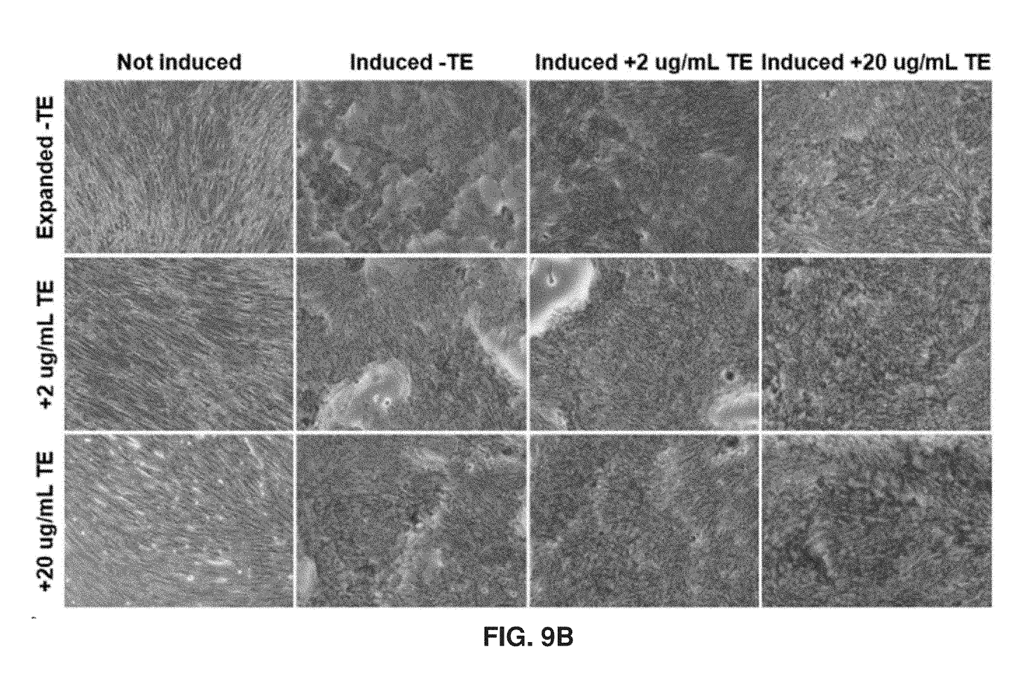

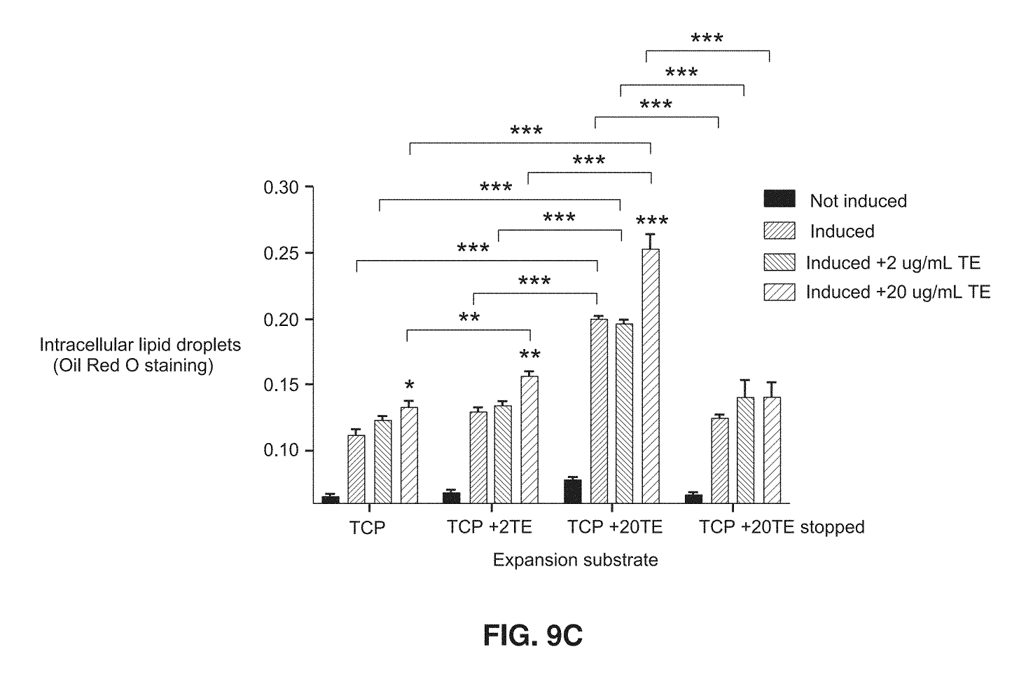

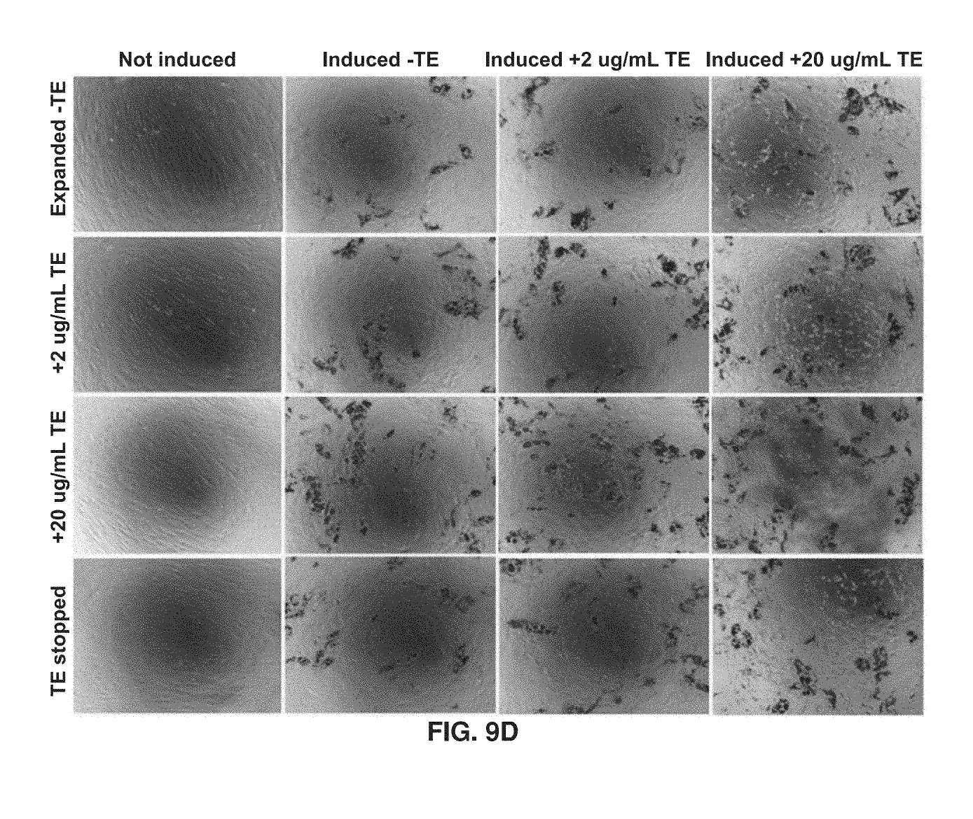

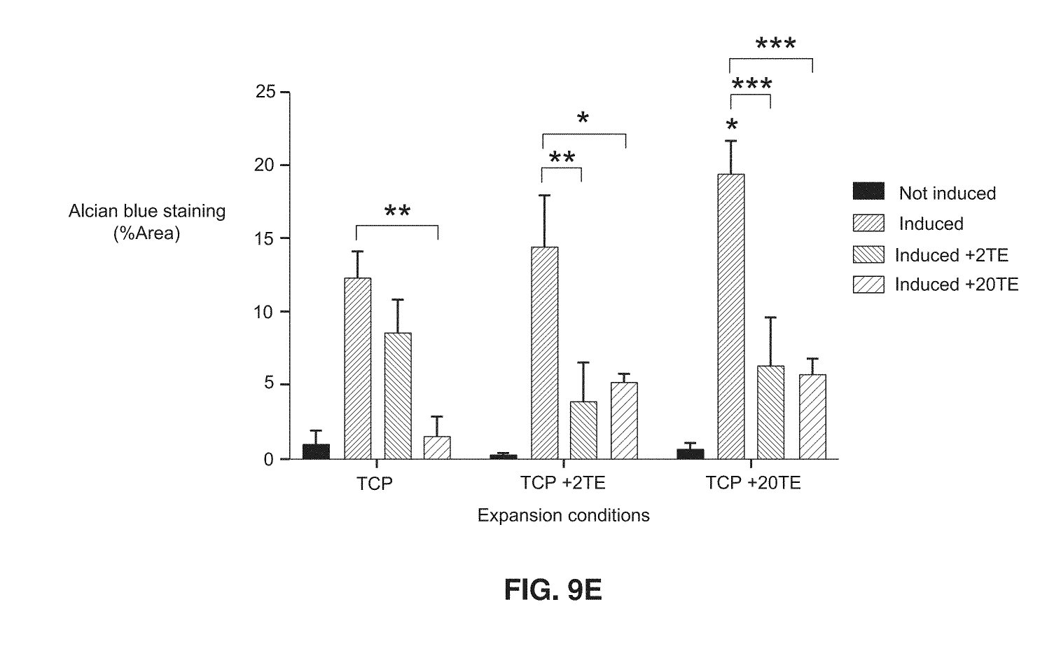

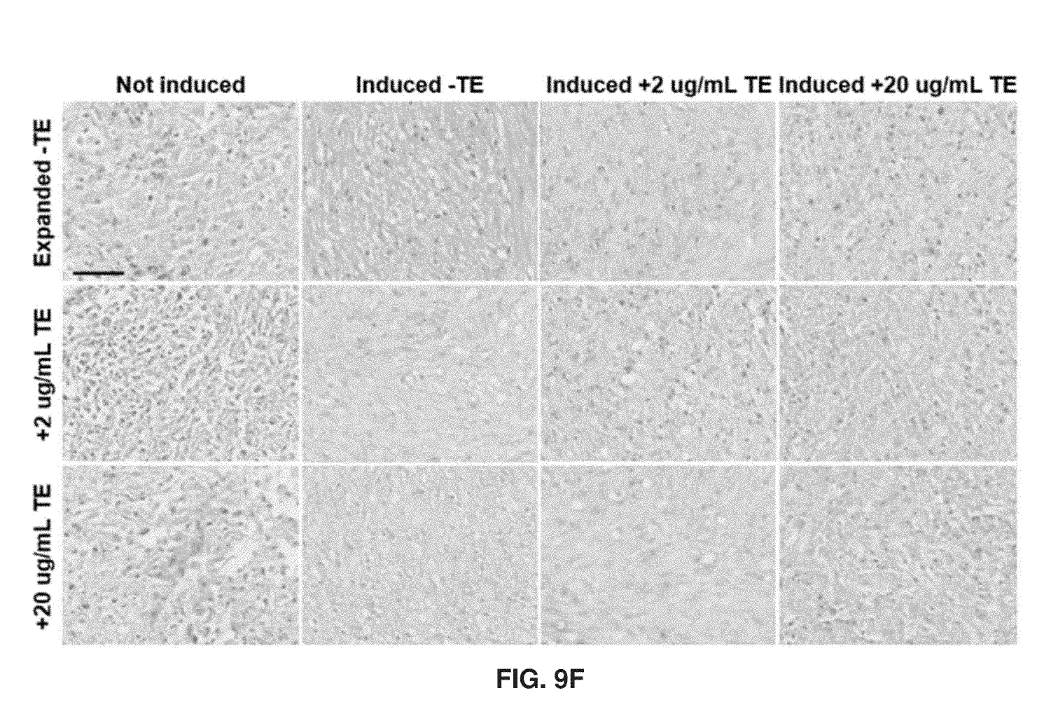

The effect of the dosing of tropoelastin on MSC osteogenesis, adipogenesis and chondrogenesis was explored. As shown in FIGS. 9A and 9B, the cells were grown and differentiated in different concentrations of TE to study the effect of TE concentration on osteogenesis. The expansion conditions comprised growth media with no TE, with 2 μg/mL TE, and with 20 ug/mL TE. The increase in mineralized calcium is indicative of osteogenesis. Maximum osteogenesis was observed when cells were grown in at least 2 μg/mL TE and induced in 20 μg/mL TE.

FIGS. 9C and 9D demonstrate the effect of tropoelastin concentrations during the expansion and induction stages in adipogenic differentiation. As shown in FIG. 9C, cells that were induced to undergo adipogenic differentiation exhibited an increase in intracellular lipid formation in the presence of TE at a concentration of 20 μg/ml TE during both expansion and induction stages.

FIGS. 9E and 9F demonstrate the effect of tropoelastin in chondrogenic differentiation. As shown in FIGS. 9E and 9F, cells expanded in 20 μg/ml TE but induced in the absence of TE exhibited the highest extent of glycosaminoglycan production. The presence of as low as 2 μg/ml TE during the induction stage significantly inhibited chondrogenic differentiation.

### Example 14

### Duration of a Cells Tropoelastin Memory

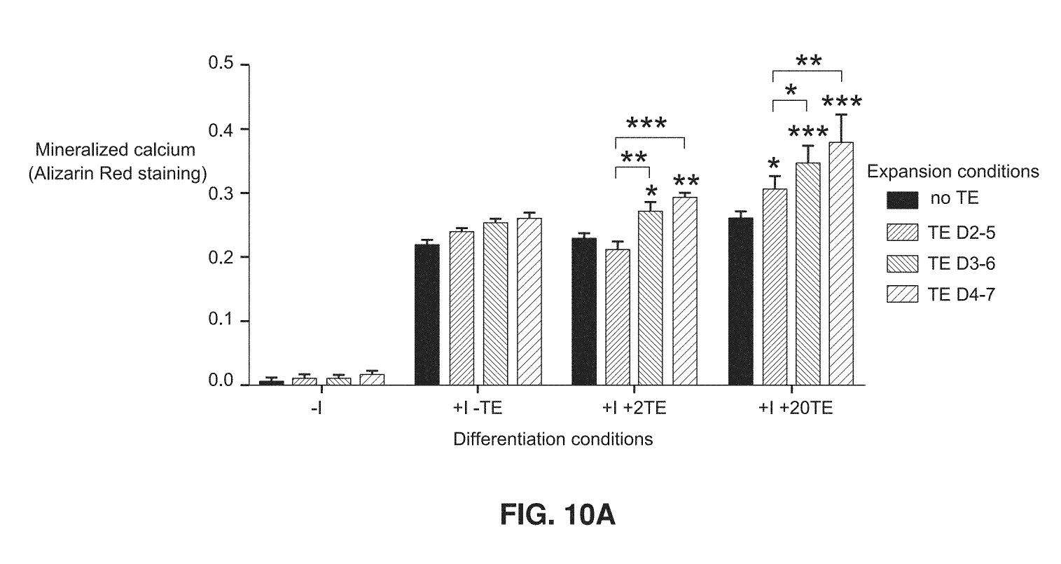

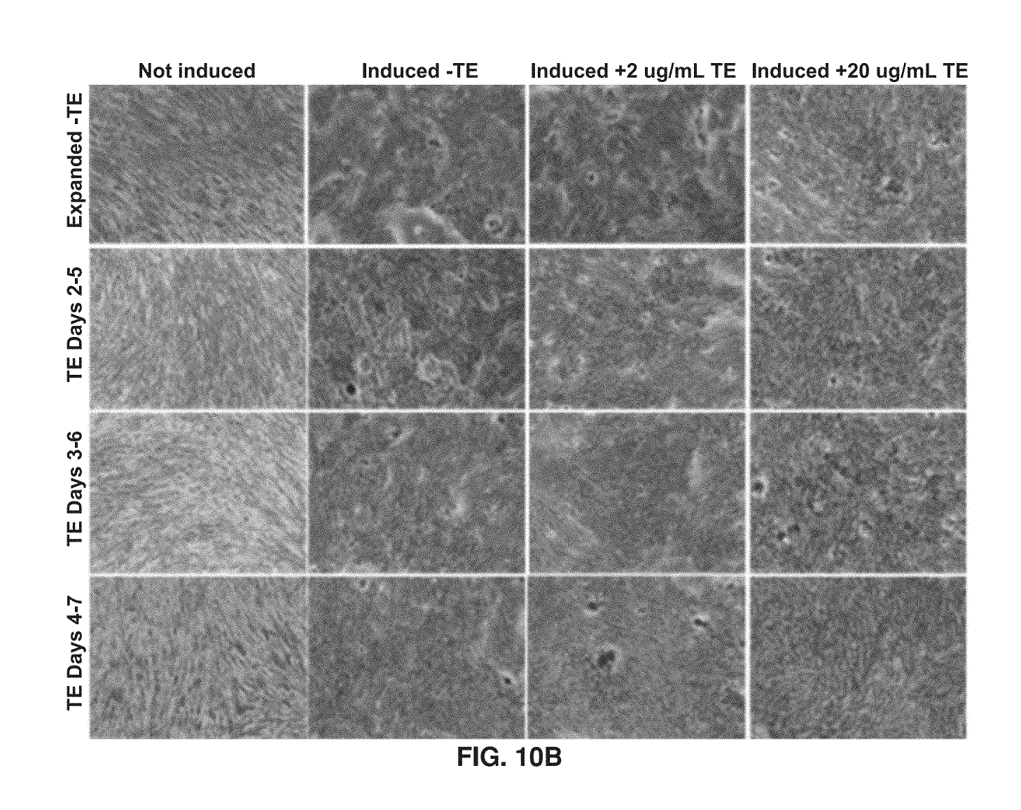

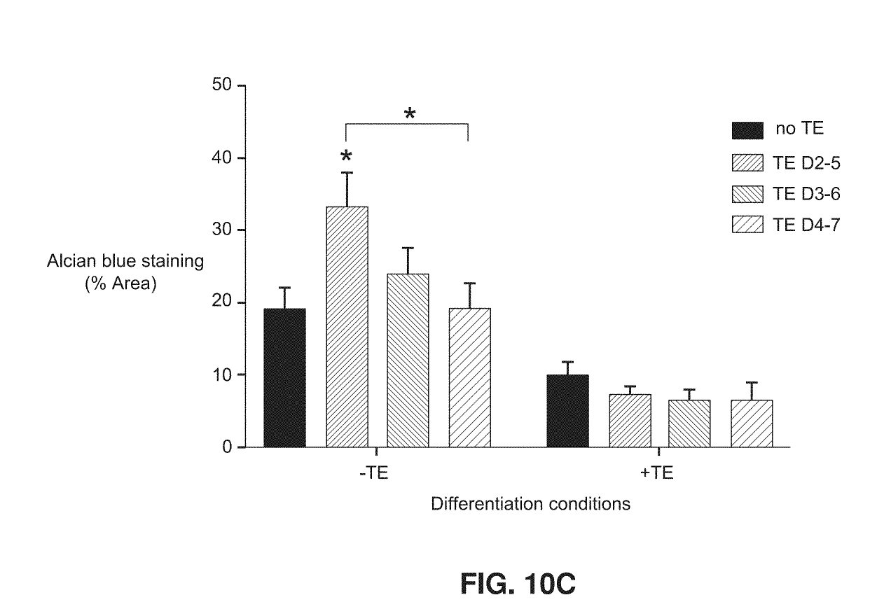

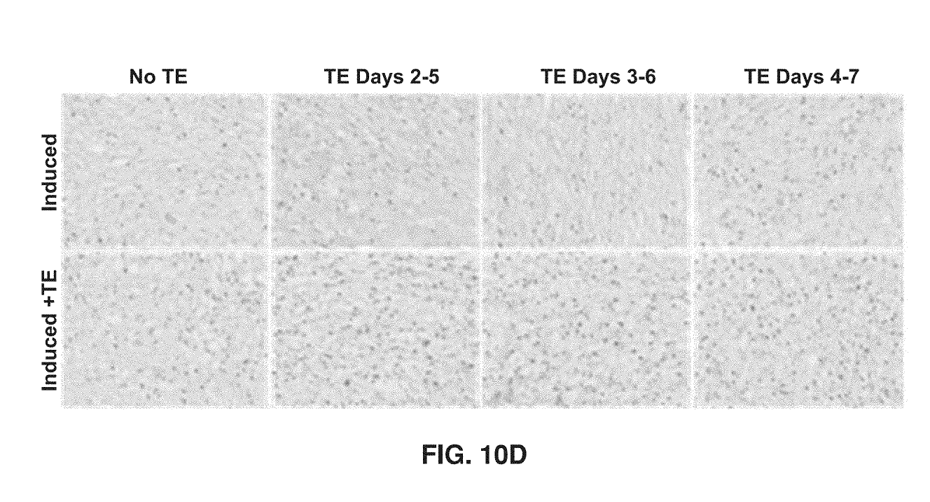

The effect of tropoelastin memory was explored during MSC osteogenesis, adipogenesis and chondrogenesis. Cells were expanded under expansion conditions (no TE, TE days 2-5, TE days 3-6 and TE days 4-7). As shown in FIGS. 10A-10B, shown for osteogenesis, the cells exhibited mineralized calcium when exposed to TE on all days during the proliferation period, with the maximum effect associated with tropoelastin exposure in the later stages of expansion. In contrast, the pro-chondrogenic effects of tropoelastin were observed only when tropoelastin was present in the early stages of expansion (FIGS. 10C-10D).

### Example 15

### Integrin Inhibition of the Tropoelastin Effects on MSC Osteogenesis

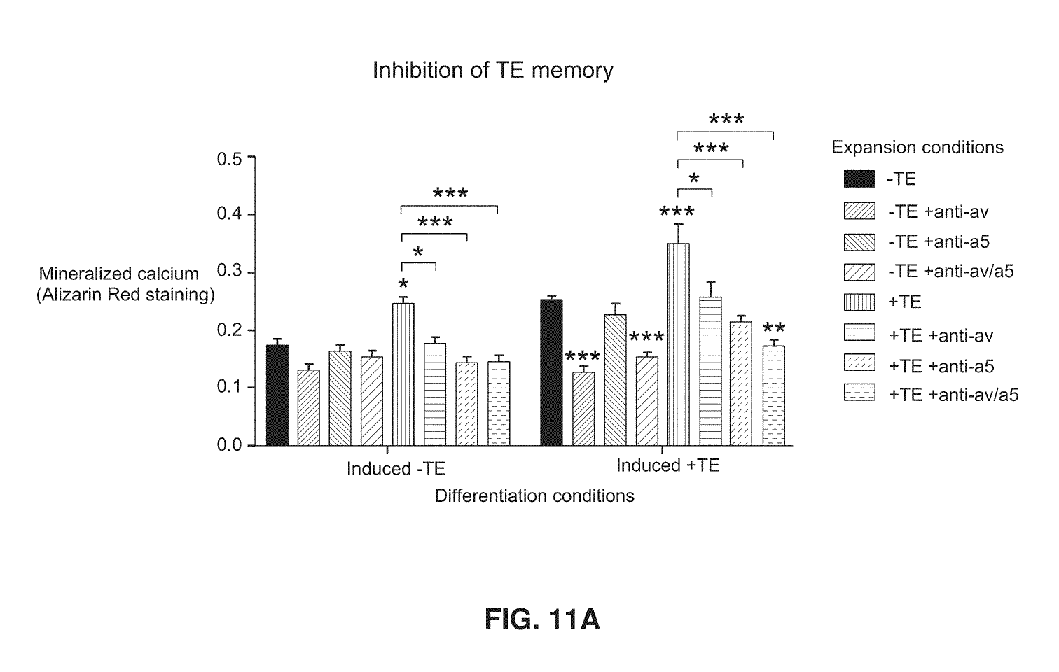

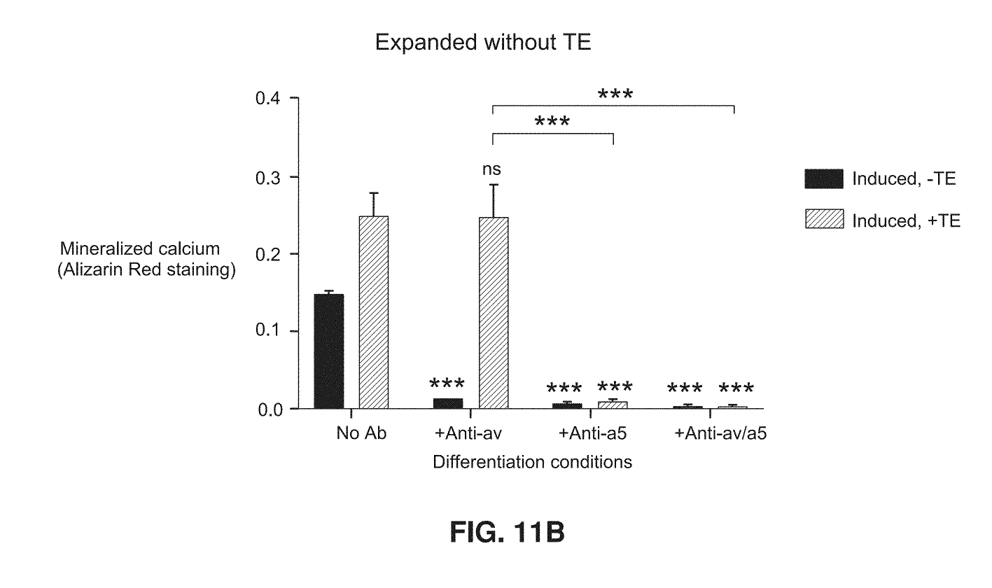

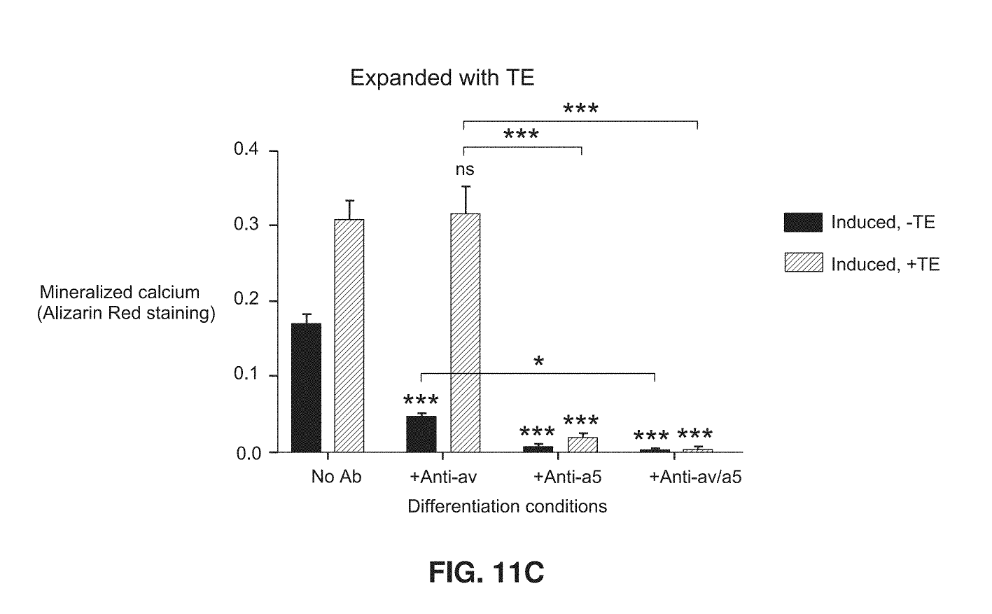

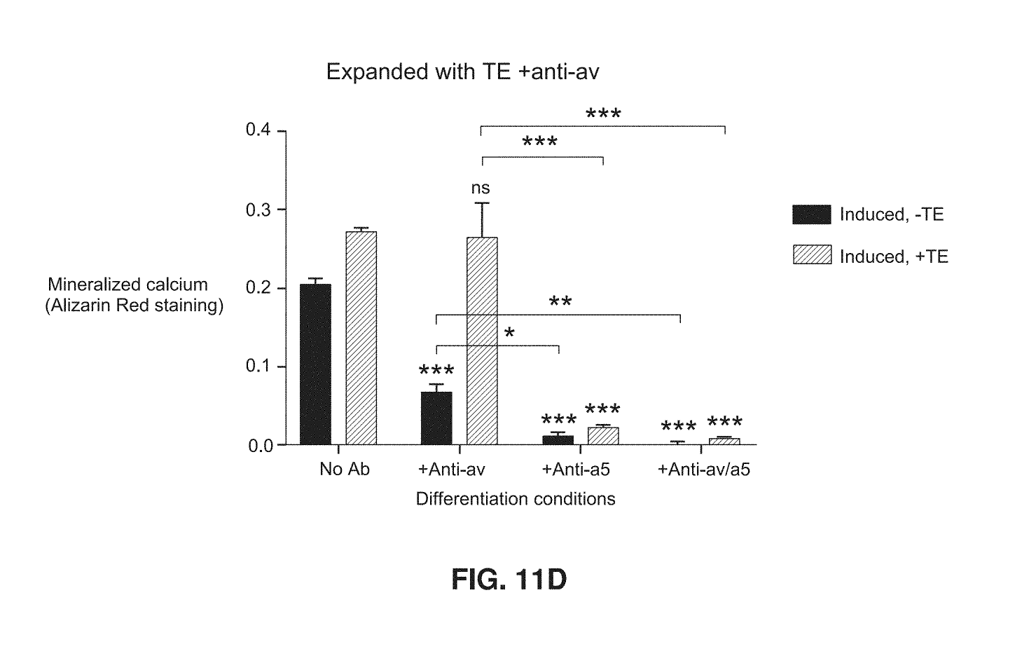

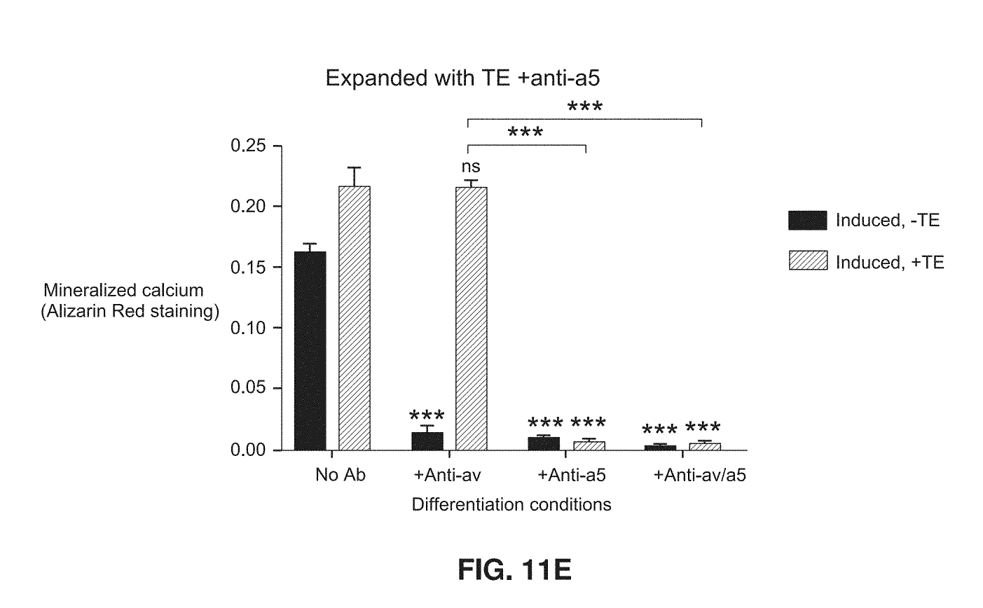

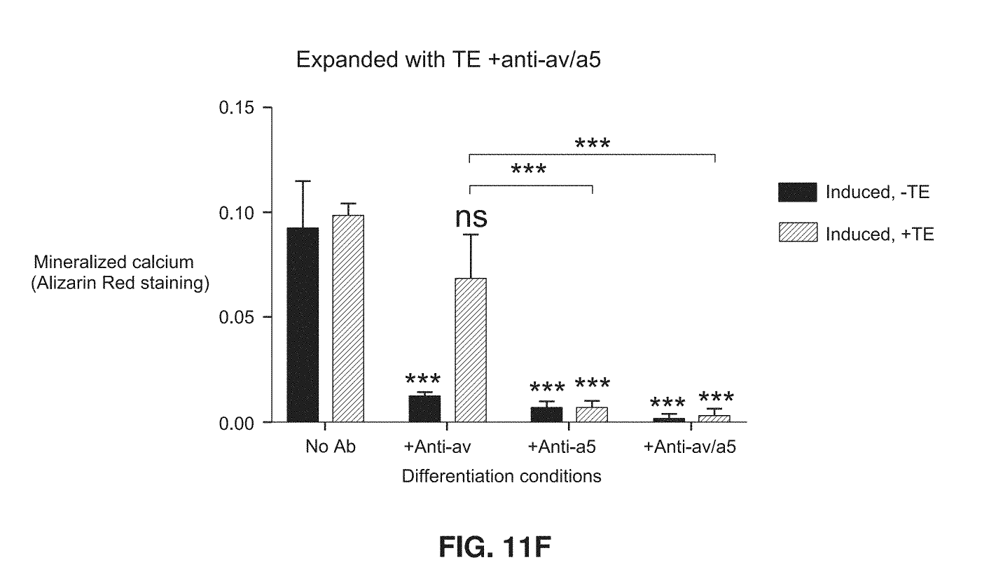

The effect of integrin inhibition of the tropoelastin on osteogenesis was also explored. As shown, cells were grown under differentiation conditions: induced without TE and induced with TE. The expansion conditions comprised: without TE; anti-av; anti-a5, anti av/a5; TE; TE with anti-av; TE with anti-a5; and TE with anti av/a5 (FIG. 11A). As shown, cells that were in the presence of tropoelastin during differentiation had higher levels of mineralized calcium. Cells that were expanded in the presence of tropoelastin with anti-av, anti-a5 or both lost this higher propensity for osteogenesis (FIG. 11A). Cells were then examined without use of TE under expansion conditions. Under the differentiation conditions of: no ab; anti-av, anti-a5 and anti-av/a5, cells were expanded without TE. As shown, cells that were differentiated without anti-av, anti-a5 or anti-av/a5 had an increase in mineralized calcium. However, cells that were induced with TE had more mineralized calcium under conditions with or without anti-av during differentiation (FIG. 11B). Cells were then expanded in the presence of TE (FIG. 11C). As shown, cells that were treated with TE with no Ab or with anti-av had increased levels of mineralized calcium, when the cells were expanded with TE as well as induced with TE (FIG. 11C). The cells were then expanded in the presence of TE and anti-av (FIG. 11D). As shown, the cells were then differentiated in the presence of no Ab, anti-av, anti-a5, or anti-av/a5. Cells that were differentiated in the presence of anti-a5 or both anti-av and anti-a5 had decreased mineralized calcium. However, cells that were expanded with both TE and anti-av and differentiated in the presence of TE and anti-av had an increase in mineralized calcium (FIG. 11D). Cells were then treated during the expansion phase with TE and anti-a5. They were then differentiated with no Ab, anti-av, anti-a5 and anti-av/a5. As shown, cells were induced with or without TE (expansion). As shown, cells that were treated with TE in the presence or absence of anti-av showed an increase in mineralized calcium (FIG. 11E). Cells were then expanded with TE and anti-av/a5 (FIG. 11F). As shown, cells were differentiated in the presence of anti-av and induced with TE led to an increase in mineralized calcium.

### Example 16

### Effects of Tropoelastin and Hyaluronic Acid on MSC Osteogenesis.

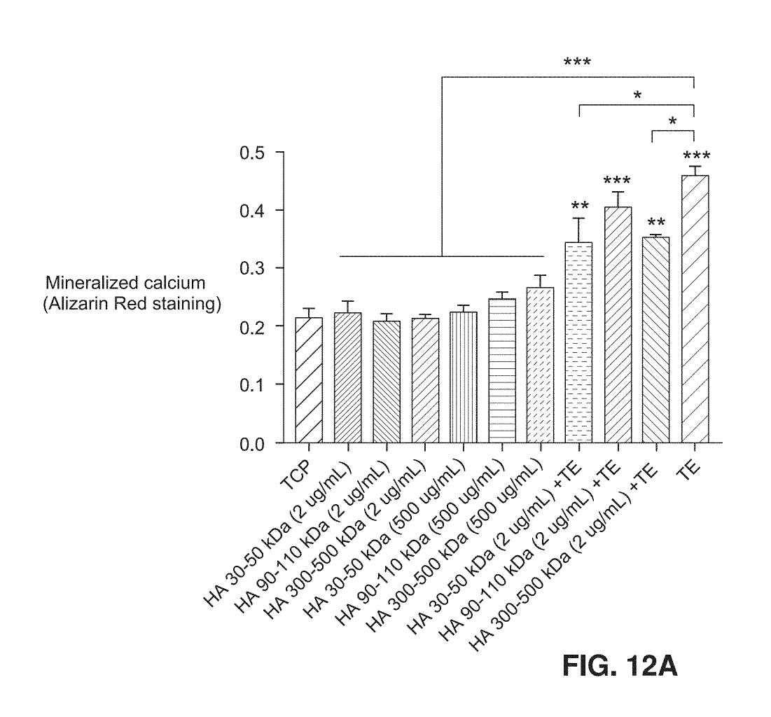

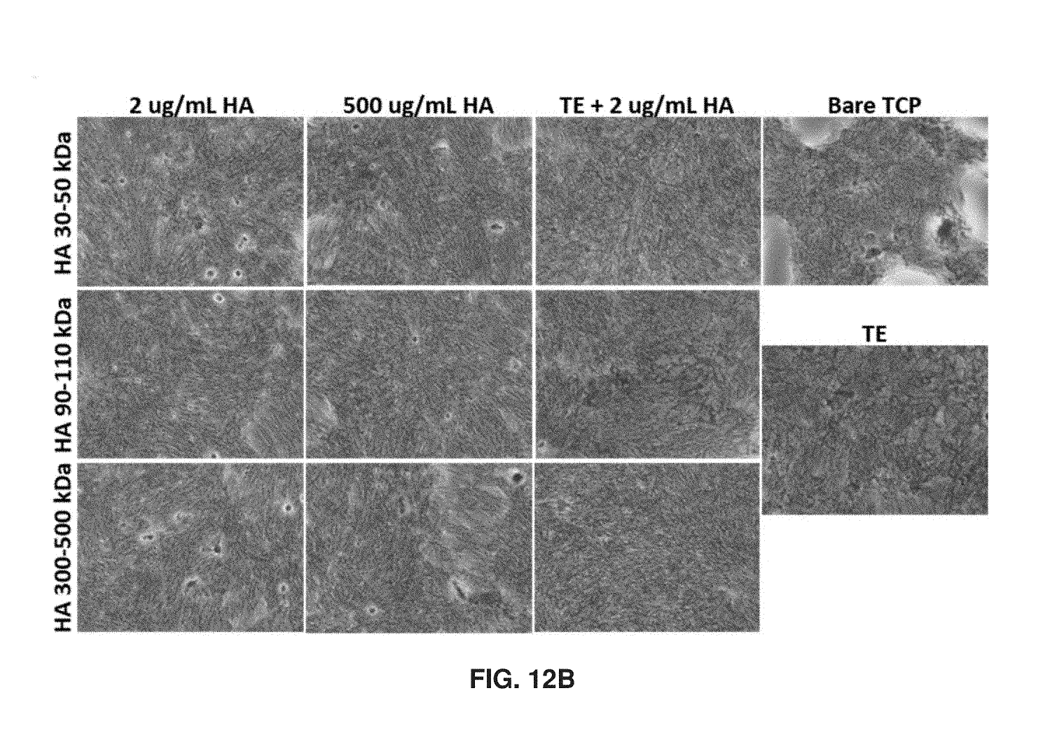

Cells were grown in the presence of different molecular weight hyaluronic acid (HA) in the absence or presence of tropoelastin. As shown, cells that were grown in the presence of tropoelastin and HA, but not HA alone, led to an increase of mineralized calcium (FIG. 12A). As shown in FIGS. 12A and 12B, shown for osteogenesis, the cells exhibited mineralized calcium when exposed to tropoelastin.

### Discussion

The ability to efficiently and cost-effectively expand therapeutic cells such as MSCs is of significant clinical and commercial interest. As with most mammalian cells, MSC proliferation is regulated by cell adhesion to the ECM and interactions with soluble factors such as cytokines, hormones and growth factors. Consequently, strategies for ex vivo MSC propagation typically graft matrix proteins on the culture substrate and/or incorporate growth factors into the culture media.

Tropoelastin by itself not only markedly augments MSC proliferation, but also parallels or surpasses the performance of specific growth factors. Among the growth factors used in MSC culture are IGF-1 and bFGF, both of which are also part of commercially available MSC growth media. As a surface coating, tropoelastin promotes cell proliferation significantly better than IGF-1, which alone does not increase cell numbers compared to normal media. This finding is consistent with reports that IGF-1 facilitates MSC migration and early-stage growth, but does not improve long-term MSC proliferation. In addition, substrate-bound tropoelastin is functionally comparable to bFGF in full serum media, and superior in reduced serum media, in stimulating a proliferative response. The high capacity of tropoelastin to stimulate proliferation allows the replacement of IGF-1 or bFGF in full serum media, and both IGF-1 and bFGF in reduced serum media, without compromising the expansion potential of MSCs. Furthermore, supplanting growth factors with a stable recombinant protein such as tropoelastin also alleviates some of the challenges associated with the use of growth factors, such as their limited availability from animal tissues⁹, high cost, and relative instability in media.

The potency of tropoelastin observed even in reduced serum media points to its potential to replace a proportion of serum during MSC culture. Serum is included in MSC growth media as it not only promotes cell attachment due to the presence of base membrane proteins such as collagens, fibronectin, laminin and vitronectin, but also induces proliferation due to growth factors, hormones and lipids^{5,8}. Therefore, the ability of tropoelastin to compensate for serum reduction is consistent with its known cell adhesive function, combined with its high mitogenic activity reflective of growth factors. Tropoelastin remarkably allows up to a 40% reduction of serum content in culture media, a unique property not exhibited by other ECM proteins. It was shown that fibronectin, which is often used as an adhesion molecule in stem cell culture, stimulated MSC proliferation similarly to tropoelastin in full serum media, but its benefits were diminished even in 20% reduced serum media.

Substantial serum compensation by tropoelastin mirrors another benefit typically associated with growth factors. As demonstrated in the examples, the ability of tropoelastin exceeds that of IGF-1 and bFGF combined. In the absence of tropoelastin, MSC proliferation is maintained in growth factor supplemented media containing 8% (v/v) FBS, but significantly decreases at 6% (v/v) FBS, which is consistent with the use of bFGF and IGF-1 with 7% (v/v) FBS in commercially available growth media (ATCC). Interestingly, inclusion of tropoelastin with both growth factors can decrease this minimum serum threshold to 6% (v/v) FBS, but only until 5 days post-seeding. Presumably, signals derived from the substrate-bound tropoelastin and soluble growth factors are propagated via alternative pathways, as defined by the relative exposure to each ligand.

The use of tropoelastin to reduce reliance on serum during MSC expansion is also clinically beneficial. Serum often can carry contaminants that pose infection risks, and as an animal-derived product, can trigger adverse immune responses²⁹. The US Food and Drug Administration and European Medicines Agency therefore recommend the avoidance of serum for culturing clinically relevant cells.

The functionality of tropoelastin, as with other matrix proteins, has conventionally been attributed to signals triggered upon cell adhesion to the molecule, whereby cell surface receptors such as integrins transduce the mechanical stimuli into chemical signals to effect a cellular response. Consistent with this paradigm, the pro-proliferative potential of tropoelastin has been ascribed solely to the elasticity, roughness and cell adhesiveness of the molecule. Accordingly, cross-linking of tropoelastin into a stiffer material abates its proliferative benefits. Contrary to this thinking, it is shown here that tropoelastin in solution above a concentration of 1 μg/mL also significantly enhances MSC expansion. At higher concentrations equivalent to the substrate coating concentration, tropoelastin in solution functionally supersedes the surface-bound protein and parallels the synergistic effect of IGF-1 and bFGF in full serum media. These findings indicate that the mitogenic activity of tropoelastin can be independent of its effect on substrate elasticity and topography. While MSC progression through the cell cycle is anchorage-dependent, cells do not need to specifically attach and spread on the effector protein such as tropoelastin for pro-proliferative signaling to occur.

Furthermore, the modulatory behavior of tropoelastin in solution is most likely independent of mechanotransductive processes. As an individual molecule, the length of tropoelastin at ˜20 nm would preclude mechanical connections with multiple cells. Within the experimental settings as described herein, tropoelastin also cannot assemble into larger cell-linking constructs, since the time scale of the proliferation assays at 7 days is significantly shorter than the minimum 12-14 days needed for elastic fibers to be formed. Also, the highest concentration of soluble tropoelastin used in these assays (20 μg/mL) is 50-fold below the critical concentration threshold for tropoelastin self-assembly.

Tropoelastin is a rare example of a full-length adhesive matrix protein that can moderate cell behavior as a soluble factor. In contrast, it was shown in the examples herein, that fibronectin in solution does not promote MSC proliferation, possibly due to poor cell recognition as its cell receptor binding sites become exposed only upon adsorption to a surface such as a collagen matrix. The effects of tropoelastin in solution are likely enabled by the inherent accessibility of its cell binding regions. Prior to this work, soluble signaling factors derived from ECM proteins, including fibronectin, laminin, collagen and elastin, are thought to be limited to peptides released by partial proteolysis, termed matrikines. Presumably, these matrikines interact with cells via proteolytically exposed cell binding motifs. As described in the examples herein, it was found that the MSC modulatory properties of tropoelastin are distinctly different from that of elastin fragments, and likely require the synergistic involvement of multiple cell-interactive regions within the full-length molecule.

The in vitro generation of MSCs can impact cell phenotype, which in turn can affect function and therapeutic potential. Therefore, it is imperative that the tropoelastin-mediated amplification of MSC proliferation does not compromise stem cell properties. As shown in the examples herein, it was found that cells expanded in the presence of substrate-bound or solution-based tropoelastin express characteristic surface markers and can undergo tri-lineage differentiation, consistent with the International Society for Cellular Therapy's definition criteria for MSCs. This ability of tropoelastin to maintain MSC phenotype during expansion equates to that of growth factors in tandem. At sufficiently high concentrations, bFGF alone preserves MSC marker expression and delays proliferation-associated changes to stemness; however, long-term use increases differentiation and decreases expression of surface markers including CD105. Consistent with this finding, it was shown in the examples as described herein, that media supplementation with IGF-1 or bFGF alone reduces levels of CD105 and/or CD73, which are expected to be constitutively expressed by MSCs. The inclusion of tropoelastin remarkably protects against this phenotypic variation within the MSC population.

Phenotypic maintenance of stem cells is signaled from either soluble factors or adhesion proteins. Prior to this work, tropoelastin has been asserted to promote stemness via MSC sensing of substrate elasticity. However, the similar protective function of tropoelastin in solution again strongly indicates an alternative anchorage-independent signaling mechanism akin to that of growth factors.

From the experiments as described herein, it was discovered that tropoelastin can directly interact with MSCs via cell surface integrins αvβ3 and αvβ5. These integrins are expressed by bone marrow derived MSCs, are recognized by distinct regions within tropoelastin, and have been implicated in tropoelastin interactions with other cell types such as fibroblasts. When activated, integrins cluster as part of focal adhesions, detected in our studies by staining for a core focal adhesion protein, vinculin. Focal adhesions link extracellular matrix proteins to the actin cytoskeleton, and transmit not only mechanical but also chemical signals from the cell environment.

While tropoelastin can directly mediate MSC attachment and spreading via integrins, the alternative hypothesis that it may elicit MSC proliferation indirectly, particularly when in solution; or directly, albeit via a non-integrin pathway, was further explored. For instance, tropoelastin may potentiate the mitogenic activity of endogenous or serum-derived growth factors such as bFGF, as many ECM proteins can bind growth factors and increase localization to their receptors. Alternatively, tropoelastin may itself activate FGFR, as intrinsic domains within some ECM proteins can serve as non-canonical ligands for growth factor receptors. In these instances, addition of the FGFR inhibitor SU-5402 should negate the pro-proliferative function of both tropoelastin and bFGF. However, MSC expansion by tropoelastin, unlike that by bFGF, was not affected beyond the non-specific inhibition associated with SU-5402 toxicity, and can therefore proceed via an FGFR-independent pathway. On this basis, the sole involvement of bFGF as the effector protein, or FGFR as the signaling receptor in tropoelastin-mediated MSC proliferation can be excluded. Moreover, antibody inhibition of tropoelastin-mediated cell proliferation indicates the participation of αv integrins, namely αvβ3 and αvβ5, in this process. Integrins have been shown to bind both immobilized and soluble ligands sufficiently to initiate signaling events, suggesting a common mechanism by which substrate-bound and soluble tropoelastin direct MSC events. However, the involvement of other cell receptors, such as the elastin binding protein, in mediating the modulatory effects of tropoelastin in solution, cannot be discounted.

A similar dual mode of action is observed in tropoelastin-directed MSC migration, in which surface tropoelastin possesses the haptotactic nature of adhesive ECM proteins, while soluble tropoelastin mirrors the chemotactic ability of chemokines and growth factors. While these signals are thought to be independent and potentially conflicting, tropoelastin can uniquely provide both biophysical and biochemical directional stimuli to elicit a potentially stronger MSC homing response. This motogenic ability of tropoelastin, which has also been reported with other cell types, can be exploited in biomedical applications to recruit resident or administered MSCs for improved therapeutic outcomes.

Tropoelastin-mediated MSC recruitment is also reliant on protein interactions with αv integrins. The abolishment of this process by antibodies that block either αvβ3 or αvβ5 strongly suggests the requisite involvement of both integrins. Integrin subunits previously implicated in MSC homing have been limited to α4, α5 or β1, and are primarily regulated by chemokine activation of cognate receptors. Tropoelastin-αv integrin interactions represent a newfound mechanism underpinning MSC migration. Furthermore, the non-inhibitory effect of αv-blocking antibodies on growth factor-mediated chemotaxis suggests separate, specific modes of MSC recruitment by tropoelastin and growth factors, at least on the cell surface level.

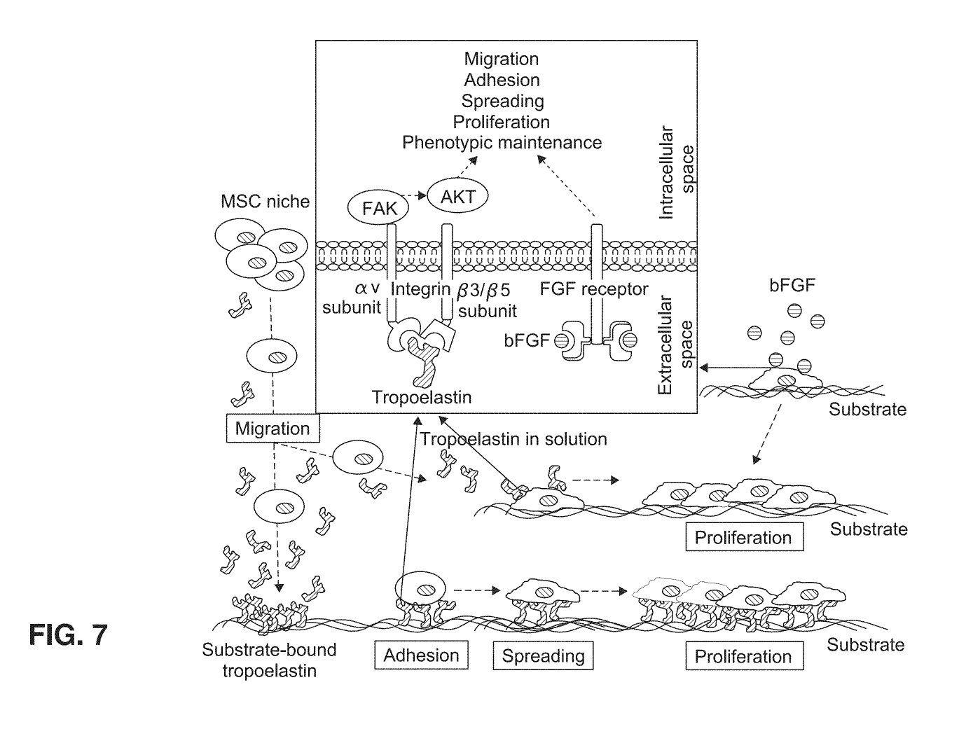

Integrin activation by ligand occupancy initiates multiple signaling cascades including serine/threonine kinase, small GTPase, and inositol lipid pathways that mediate cell survival, adhesion, spreading, proliferation and migration. Several of these pathways are also activated by bFGF binding to its FGF receptor in MSCs. Furthermore, the association of αv integrins with growth factor receptors is thought to be required for sustained growth factor activation of downstream proliferative signals. In support, blocking αv integrins inhibits cell growth even in the presence of growth factors, which reflects our findings that αv integrin inhibition also attenuates bFGF-mediated MSC expansion. The overlap of intracellular signaling cascades shared by integrins and FGF receptors represents a possible mechanism by which tropoelastin parallels, and can therefore replace, the mitogenic, protective and motogenic functions of growth factors such as bFGF (FIG. 7).

The functionalities of tropoelastin, particularly in terms of MSC migration, propagation, growth factor replacement and serum compensation, appear to be unique to this protein, despite the similar ability of other ECM proteins to bind integrins. It is thought that not all ECM-integrin interactions promote cell cycle progression equally, despite similar capabilities for cell adhesion and cytoskeletal organization. For example, αvβ3 integrin can specifically associate with adapter proteins downstream of growth factor receptors and cooperatively activate and sustain long-term mitogenic pathways, allowing αvβ3 ligands such as tropoelastin to enhance cell proliferation more potently than non-ligands. Moreover, matrix proteins such as fibronectin may adhere up to 20 types of integrins, which can drive opposing effects on cell proliferation and attenuate or avert the target cell response. The narrow integrin selectivity of tropoelastin may therefore contribute to its specific outcomes on MSC behavior.

The potent mitogenic and motogenic effects of tropoelastin on MSCs is surprising, since it is not natively present in the stem cell niche unlike bFGF. It is proposed that this growth factor-like behavior of tropoelastin becomes biologically relevant in instances requiring rapid MSC homing and elevated MSC proliferation; namely, during embryonic development and wound repair, which coincide with the only periods in which free tropoelastin abound in the extracellular environment. During the fetal to neonatal stages, peak tropoelastin synthesis occurs alongside widespread bFGF expression, which may recruit MSCs and drive their propagation for normal development. The known inhibitory effects of bFGF on tropoelastin production during development may indeed be a regulatory mechanism to safeguard against uncontrolled stem cell numbers resulting from the cumulative effects of bFGF and tropoelastin. During injury, upregulated tropoelastin secretion may supplement the low level of bFGF in tissues, to rapidly stimulate MSC migration and proliferation integral to wound healing.

### Materials and Method

Cell Culture

Human bone marrow-derived MSCs obtained from American Type Culture Collection (ATCC) were cultured in normal media, which consists of Alpha-Minimum Essential Medium (α-MEM) (Lonza) with 10% (v/v) FBS (Life Technologies) and 2.4 mM L-glutamine (Lonza), at 37° C. in a humidified normoxic incubator up to a maximum of 10 population doublings. Where indicated, the normal media was supplemented with 15 ng/mL IGF-1 (Life Technologies) and/or 125 μg/mL bFGF (Life Technologies), equivalent to the growth factor concentrations in the ATCC-recommended media. Cells were passaged once they reach 70-80% confluence.

Substrate Coating with ECM Proteins

Where indicated, tissue culture plastic wells were coated with 20 μg/mL recombinant human tropoelastin (Elastagen) or 2 μg/mL fibronectin (Sigma-Aldrich®) in PBS (10 mM phosphate, 150 mM NaCl, pH 7.4) at 4° C. overnight. The protein solution was removed, and wells were washed three times with PBS to remove unbound protein prior to cell seeding.

Media Supplementation with ECM Proteins

Where indicated, normal media was supplemented with 2.5-20 μg/mL tropoelastin (Elastagen), 2.5-50 μg/mL of κELN (soluble human skin elastin from Elastin Products Company), or 2.5-50 μg/mL αELN (soluble human lung elastin from Elastin Products Company). To prevent protein adhesion on the tissue culture substrate, wells were pre-incubated with normal media for 5 hours to enable surface blocking by serum proteins prior to cell seeding in supplemented media.

To confirm surface blocking, tropoelastin was added to pre-incubated or bare well surfaces for 1 hour at room temperature. Excess protein was removed with three PBS washes. Levels of bound tropoelastin were detected via an enzyme-linked immunosorbent assay, using 1:2000 mouse anti-elastin BA4 primary antibody (Sigma-Aldrich®) for 1 hour at room temperature, 1:5000 goat anti-rabbit IgG horseradish peroxidase-conjugated secondary antibody (Sigma-Aldrich®) for 1 hour at room temperature, and visualized with 40 mm 2,2′-azino-bis(3-ethylbenzthiazoline-6-sulfonic acid) (ABTS) (Sigma-Aldrich®) solution in 0.1 mM sodium acetate, 0.05 mM NaH₂PO₄, pH 5 containing 0.01% (v/v) H₂O₂ for 1 hour at room temperature. Sample absorbances were read at 405 nm.

Cell Proliferation

Sub-confluent flasks of MSCs were treated with 0.05% (v/v) trypsin-EDTA (Sigma-Aldrich®) at 37° C. for 5 min to lift off adherent cells from the culture vessel. Trypsin was neutralized with two volumes of serum-containing growth media. Cells were centrifuged at 270 g for 5 min and resuspended in the required media. Cells were seeded at a density of 5000 cells/cm² on bare or protein-coated tissue culture plastic wells, in normal or supplemented media. Media was changed every 2 days. After specific time points, cells were fixed with 3% (v/v) formaldehyde at room temperature for 20 min, washed with PBS, then stained with 0.1% (w/v) crystal violet in 0.2 M MES buffer for 1 hour. Excess stain was washed off four times with reverse osmosis water. The retained stain was solubilized with 10% (v/v) acetic acid and sample absorbance values indicative of cell abundance were read at 570 nm. Sample absorbance values were subtracted by baseline values (corresponding to cell numbers in serum-free media, or cell numbers on day 1 post-seeding) and expressed as a fraction of the highest absorbance among all samples on day 7 post-seeding.

EDTA Inhibition

MSCs were seeded at a density of 1.5×10⁵ cells/cm² on tropoelastin-coated wells in serum-free α-MEM containing 0-9 mM ethylenediaminetetraacetic acid (EDTA) (Sigma-Aldrich®). The cells were incubated for 1 hour at 37° C., then washed with PBS to remove unbound cells. Bound cells were fixed, stained and measured for absorbance at 570 nm as described for the proliferation assays. The percentage of cell attachment was determined relative to a set of standards with known cell numbers.

Cation Add-Back

MSCs were washed with cation-free PBS, centrifuged at 270 g for 5 min, and resuspended in cation-free PBS. The cells were seeded at a density of 1.5×10⁵ cells/cm² on tropoelastin-coated wells in the presence of 0-0.5 mM cation (Mg²⁺, Ca²⁺ or Mn²⁺) and incubated for 45 min at 37° C. Bound cells were fixed and stained, and cell attachment was quantified as previously described.

Cell Spreading

MSCs were seeded at a density of 7.5×10⁴ cells/cm² on tropoelastin-coated wells in serum-free α-MEM for 1.5 hour at 37° C. Cells were fixed and visualized by phase contrast microscopy with a Zeiss Axio Vert.A1 microscope. Images were taken on an AxioCam ICml monochrome camera. Cells were categorized as spread, i.e. cells which exhibit a phase-dark, flattened morphology, or unspread, i.e. cells which appear round and phase-bright. Cell spreading was quantified by counting the percentage of spread cells in each field of view. Three fields of view were obtained for each sample replicate.

Immunofluorescent Staining

MSCs were seeded on TCP coated with 20 μg/mL tropoelastin or 10 mg/mL BSA for 1 day. Focal adhesions were detected with a fluorescently-tagged anti-vinculin monoclonal antibody, while cell nuclei were stained with DAPI using the Focal Adhesion Staining Kit (Merck Millipore). Samples were visualized and imaged with an Olympus FV1000 confocal microscope at the Australian Centre for Microscopy & Microanalysis, University of Sydney. Focal adhesion density per cell was calculated by dividing the number of pixels corresponding to stained vinculin by the number of cells in each field of view, then averaged for each sample.

Integrin and FGFR Inhibition

To block specific integrin activity, up to 20 μg/mL of anti-αv or anti-αvβ3 integrin antibodies (Abcam®), or up to 1:250 dilution of anti-αvβ5 integrin antibody (Abcam®) was added to the media during MSC spreading or proliferation assays. Optimal inhibitory concentrations were selected for the anti-αv (5 μg/mL), anti-αvβ3 (5 μg/mL), and anti-αvβ5 (1:500 dilution) integrin antibodies. An anti-β8 integrin (5 μg/mL) (Abcam®) or a non-specific mouse IgG (5 μg/mL) (Sigma-Aldrich®) were also included as negative antibody controls. To block FGFR activity, up to 20 μM of the SU-5402 FGFR inhibitor (Sigma-Aldrich®) was added to the media during MSC proliferation. The integrin and FGFR inhibitors were replenished during every media change.

Cell Migration via Haptotaxis

Polydimethylsiloxane (PDMS) was casted into 3D printed molds to create a circular shape with three rectangular cutouts, in which the middle rectangle was equidistant from the flanking rectangles. The PDMS stencil was placed inside a well plate and pressed tightly against the well surface to create watertight seal. The top and bottom rectangular chambers were filled with tropoelastin solution (20 μg/mL) or PBS, respectively, and air dried overnight. MSCs (1.2×10⁶ cells/cm²) were seeded into the middle chamber and allowed to attach for at least 2 hrs. The PDMS stencil was removed, and the culture well was covered with normal media. For up to 5 days, cells were stained daily with NucBlue™ Live ReadyProbes™ Reagent (Life Technologies) for 15 min, washed once with PBS, covered with normal media, and imaged under fluorescence at 360/460 nm using a Nikon Ti-E Live Cell Microscope. Cell migration into regions defined by tropoelastin or PBS coating was quantified via relative fluorescent areas using ImageJ software.

Cell Migration via Chemotaxis

Chemotaxis was measured using a fluorimetric 96-well Boyden chamber assay system (QCM Chemotaxis Cell Migration Assay, Millipore) according to the supplier's instructions. Normal, tropoelastin-supplemented, or growth factor-supplemented media was added to the lower chamber of the well plate, while MSCs were seeded at 14,300 cells/cm² in normal media into the upper chamber. Where indicated, integrin-blocking antibodies were added at optimized concentrations to the upper chamber with the cells. Cells that migrate through the permeable membrane into the lower chamber were detached and quantified. Normalized cell migration was calculated by subtracting the extent of undirected cell migration (where no chemoattractant was added to the lower chamber) from each experimental result.

Flow Cytometry

MSCs cultured for 5 or 7 days in various media formulations and on bare or protein-coated tissue culture wells were trypsinised and pelleted. The cell pellets were washed with 0.22 μm filtered FACS buffer (5% v/v FBS in PBS) and re-centrifuged at 270 g for 5 min. The cells were resuspended in FACS buffer to a concentration of 100,000 cells in 100 μL total volume, and probed for MSC marker expression using the Human Mesenchymal Stem Cell Verification Flow Kit (R&D Systems®). Isotype and unstained control samples were prepared using MSCs cultured in standard growth media on tissue culture plastic. Cells were analyzed using a BD™ Biosciences LSR II Flow Cytometer System. Singlet cells were determined by their forward scatter-to-side scatter and scatter height-to-width ratios, while viable cells were identified by exclusion of 1:20 propidium iodide. Only singlet, viable cells were analyzed for marker expression.

Cell Differentiation

MSCs were grown in various media formulations and on bare or protein-coated tissue culture wells for 7 days. The expanded cells were harvested, re-seeded on TCP, and differentiated into the adipogenic, osteogenic and chondrogenic lineages using the hMSC Adipogenic BulletKit®, hMSC Osteogenic BulletKit®, and hMSC Chondrogenic BulletKit® (Lonza), respectively, following the manufacturer's instructions.

To confirm adipogenesis, cells that had been induced for 25 days were washed with PBS, fixed with 10% (v/v) formalin for 30 min, then washed with water. Cells were incubated with 60% (v/v) isopropanol for 5 min and stained for intracellular lipid droplets with 1.8 mg/mL Oil Red O in isopropanol for 20 min. Excess stain was removed with 5 washes of water.

To confirm osteogenesis, cells that had been induced for 14 days were fixed and stained for mineralized calcium deposits with Alizarin Red S, as previously described. Cells from the adipogenic and osteogenic experiments were imaged with a Zeiss Axio Vert.A1 microscope using an AxioCam 105 colour camera.

To confirm chondrogenesis, cell pellets that had been induced for 14 days were washed with PBS, embedded in 1.5% (w/v) agar containing 0.85% (w/v) NaCl, and fixed with 10% (v/v) formalin overnight. The samples were dehydrated in 70% (v/v) ethanol for 1 day, then paraffin-embedded, sectioned, mounted onto slides, stained with Alcian Blue (pH 2.5) for 1 hour and counterstained with Nuclear Fast Red. Samples were imaged with an Olympus VS120 Slide Scanner.

Statistical Analyses

All data were reported as mean±standard error of the mean (n=3). Statistical comparisons were calculated using analysis of variance (ANOVA). Significance was set at p<0.05. Statistical significance was denoted in figures by asterisks: * (p<0.05), ** (p<0.01), or *** (p<0.001).

Summary of Results

Tropoelastin Effects on Mesenchymal Stem Cell (MSC) Differentiation

Exposure to tropoelastin during MSC expansion and induction modulates the cells' functional differentiation into bone, fat and cartilage.

The presence of tropoelastin during MSC expansion improved osteogenic potential by 42%. Tropoelastin addition during differentiation improved osteogenesis by 55%. Tropoelastin addition during both expansion and differentiation stages further increased osteogenesis by up to 131%.

Tropoelastin addition during MSC expansion or differentiation increased adipogenesis by 33% and 19%, respectively. Tropoelastin addition during both the expansion and differentiation stages promoted adipogenesis by 69-85%, with greater benefits associated with an uninterrupted tropoelastin presence.

Similarly, tropoelastin addition during MSC expansion improved chondrogenesis by 134%. In contrast, tropoelastin addition during chondrogenesis effectively inhibited this process, regardless of tropoelastin presence during the expansion stage. Tropoelastin addition during differentiation decreased MSC chondrogenesis by 63% (if cells were expanded without tropoelastin) to 80% (if cells were expanded with tropoelastin).

Tropoelastin Memory

Prior exposure to tropoelastin during MSC expansion has a lingering effect on tri-lineage differentiation.

For osteogenesis, a smaller temporal gap (maximum 2 days) between tropoelastin exposure during expansion and differentiation results in a better outcome. MSCs exposed to tropoelastin from days 4-7 of a seven-day expansion period displayed 24% increased osteogenesis compared to cells exposed from days 2-5.

For chondrogenesis, exposure to tropoelastin during the early stage of expansion improves outcomes. MSCs exposed to tropoelastin from days 2-5 of the expansion period showed 71% increased chondrogenesis compared to cells exposed from days 4-7.

Inhibition of Tropoelastin Effects

The pro-osteogenic effects of tropoelastin during MSC expansion are mediated by alpha v and alpha 5 integrins. The inclusion of anti-alpha v, alpha 5, or alpha v and alpha 5 integrin antibodies with tropoelastin during MSC expansion attenuated the promotion of osteogenesis by 28%, 41%, and 40%, respectively, when cells were induced without tropoelastin; and by 26%, 39% and 50%, respectively, when cells were induced with tropoelastin.

The inclusion of one or both anti-integrin antibodies during MSC differentiation impeded the cells' ability to undergo osteogenesis. However, when cells were induced in the presence of tropoelastin, addition of the anti-alpha v integrin antibody did not affect MSC osteogenesis, indicating that the pro-osteogenic effect of tropoelastin during MSC differentiation does not require alpha v integrins.

Pro-Osteogenic Effects of Tropoelastin vs Hyaluronic Acid

In formulations containing tropoelastin and hyaluronic acid, tropoelastin is the dominant promoter of MSC osteogenesis. Cells grown on a coating of 90% tropoelastin and 10% hyaluronic acid displayed 60-88% increased osteogenesis compared to cells grown on TCP. Cells grown on tropoelastin alone displayed 113% higher osteogenesis, while cells grown on hyaluronic acid alone showed similar levels of osteogenesis to those grown on TCP.

It will be understood that the embodiments disclosed and defined in this specification extends to all alternative combinations of two or more of the individual features mentioned or evident from the text or drawings. All these different combinations constitute various alternative aspects of the disclosed embodiments.

1. A method for forming cells of mesodermal lineage from mesenchymal stem cells (MSC) comprising:
contacting MSCs with:
(i) at least one differentiation factor for inducing formation of cells of mesodermal lineage from MSC; and
(ii) tropoelastin,
wherein the number of cells of mesodermal lineage formed from MSC in the presence of tropoelastin is greater than the number of cells of mesodermal lineage formed in the absence of tropoelastin,
thereby forming cells of mesodermal lineage from MSCs.

2. The method of claim 1, wherein the tropoelastin is arranged on a cell culture surface of a cell culture vessel to enable the MSCs to contact the tropoelastin when the MSCs are contacted with the cell culture surface.

3. The method of claim 1, wherein the tropoelastin is partially or fully solubilized in a cell culture medium for culture of an MSC.

4. The method of claim 1, wherein the method further comprises:
(i) contacting MSCs with tropoelastin in the absence of factors that induce differentiation to induce proliferation of MSCs, thereby forming a population of MSCs; and
(ii) contacting the population of MSCs with at least one differentiation factor for inducing formation of cells of mesodermal lineage from MSC and tropoelastin.

5. The method of claim 1, wherein the method further comprises:
(i) culturing MSCs in a first medium containing tropoelastin to form a tropoelastin-cultured MSC population; and
(ii) culturing said tropoelastin-cultured MSC population in a second medium, wherein the second medium includes at least one differentiation factor for inducing differentiation of an MSC.

6. The method of claim 1, wherein the tropoelastin is provided in the form of a complex with hyaluronic acid that is partially or completely soluble, wherein the tropoelastin monomers are linked together by hyaluronic acid.

7. The method of claim 6, wherein the tropoelastin is cross-linked to the hyaluronic acid.

8. The method of claim 1, wherein the cell of mesodermal lineage is an osteocyte, chondrocyte or adipocyte.

9. The method of claim 1, wherein the MSCs are human MSCs.

10. A composition of cells formed by the method of claim 1.

11. The composition of claim 10, wherein the composition is a substantially pure form of osteocytes.

12. The composition of claim 10, wherein the composition includes tropoelastin and/or hyaluronic acid.

13. The composition of claim 12, wherein the tropoelastin is cross-linked to the hyaluronic acid.

14. A method for treating an individual having a bone disorder or fracture comprising:
providing a composition according to claim 11 to the individual, thereby treating the individual for a bone disorder or fracture.

15. The method of claim 14, wherein the individual is provided the composition, wherein the amount of total MSC provided to the individual in the composition is at least one to two million cells per kilogram of body weight of the individual or wherein the amount of total MSC provided to the individual in the composition is at least one to two million cells, and wherein the composition is administered to a local site.

16. A cell culture medium comprising tropoelastin, wherein the cell culture medium does not contain insulin-like growth factor-1 (IGF-1) and/or basic fibroblast growth factor growth factor (bFGF).

17. The cell culture medium of claim 16, wherein the cell culture medium comprises about about 2.5 μg/mL to about 20 μg/mL tropoelastin.

18. The cell culture medium of claim 16, wherein the cell culture medium comprises about 2% to about 10% serum or about 2% to about 6% serum.

19. The cell culture medium of claim 18, wherein the serum is fetal bovine serum (FBS).

20. The cell culture medium of claim 16, wherein the cell culture medium is serum-free.

21. The cell culture medium of claim 16, wherein the cell culture medium comprises about 2.5 μg/mL to about 20 μg/mL tropoelastin, about 2% to about 10% FBS, minimal essential medium (MEM), and L-glutamine.

22. The cell culture medium of claim 16, wherein the tropoelastin is provided in the form of a complex with hyaluronic acid.

23. The cell culture medium of claim 22, wherein the tropoelastin is cross-linked to the hyaluronic acid.

24. A cell culture comprising:
mesenchymal stem cells; and
a medium comprising tropoelastin, wherein the medium does not contain insulin-like growth factor-1 (IGF-1) and/or basic fibroblast growth factor growth factor (bFGF).

25. The cell culture of claim 24, wherein the mesenchymal stem cells are human mesenchymal stem cells.

26. The cell culture of claim 24, wherein the medium comprises about 2.5 μg/mL to about 20 μg/mL tropoelastin.

27. The cell culture of claim 24, wherein the tropoelastin is provided in the form of a complex with hyaluronic acid.

28. The cell culture of claim 24, wherein the tropoelastin is cross-linked to the hyaluronic acid.

29. The cell culture of claim 24, wherein the medium comprises 2% to about 10% serum or about 2% to about 6% serum.

30. The cell culture of claim 24, wherein the medium is serum-free.

31. The cell culture of claim 24, wherein the medium comprises about 2.5 μg/mL to about 20 μg/mL tropoelastin, about 2% to about 10% FBS, minimal essential medium (MEM), and L-glutamine.
